(19) United States
(12) Patent Application Publication
(10) Pub. No.: US 2024/0210407 A1
(43) Pub. Date: Jun. 27, 2024

(54) COMPOSITIONS AND METHODS FOR DETECTING AND TREATING PATHOLOGICAL FIBROBLAST CELLS

(71) Applicant: Duke University, Durham, NC (US)

(72) Inventors: Paul Ferrell, Durham, NC (US); Kristianne Oristian, Durham, NC (US); Salvatore Pizzo, Durham, NC (US)

(21) Appl. No.: 18/392,091

(22) Filed: Dec. 21, 2023

Related U.S. Application Data
(60) Provisional application No. 63/434,565, filed on Dec. 22, 2022.

(51) Int. Cl. G01N 33/577; A61K 45/06; A61P 9/00; C07K 16/28; G01N 33/569; A61K 39/00

(52) U.S. Cl. CPC G01N 33/577; A61K 45/06; A61P 9/00; C07K 16/2896; G01N 33/56966; A61K 2039/505

(57) ABSTRACT

The present invention provides compositions and methods for detecting conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) in a subject through detecting aberrant pro-N-cadherin (PNC) cellular localization and/or PNC circulation in blood (e.g., a blood, plasma, serum, a whole blood, buffy coat). In addition, the present invention provides methods for treating conditions characterized with pathological fibroblasts (e.g., heart failure) (e.g., early stages of heart failure) (e.g., cardiomyopathy) (e.g., idiopathic pulmonary fibrosis) (e.g., nonalcoholic fatty liver disease with cirrhosis) in a subject through inhibiting aberrant PNC cellular localization and/or PNC circulation in blood (e.g., a blood, plasma, serum, a whole blood, buffy coat).

### CROSS-REFERENCE TO RELATED APPLICATIONS

This application claims the benefit of U.S. Patent Application No. 63/434,565, filed Dec. 22, 2022, the contents of which is incorporated by reference herein in its entirety.

### REFERENCE TO A SEQUENCE LISTING

The text of the sequence listing submitted Dec. 21, 2023, titled “DUKE_41521_202_SequenceListing.xml”, created Dec. 21, 2023, having a file size of 2,085 bytes, is hereby incorporated by reference in its entirety.

### FIELD OF THE INVENTION

The present invention provides compositions and methods for detecting conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) in a subject through detecting aberrant pro-N-cadherin (PNC) cellular localization and/or PNC circulation in blood (e.g., a blood, plasma, serum, a whole blood, buffy coat). In addition, the present invention provides methods for treating conditions characterized with pathological fibroblasts (e.g., heart failure) (e.g., early stages of heart failure) (e.g., cardiomyopathy) (e.g., idiopathic pulmonary fibrosis) (e.g., nonalcoholic fatty liver disease with cirrhosis) in a subject through inhibiting aberrant PNC cellular localization and/or PNC circulation in blood (e.g., a blood, plasma, serum, a whole blood, buffy coat).

### INTRODUCTION

Fibrosis is the formation of excess fibrous tissue in an organ or tissue, often as a reaction to inflammation or tissue injury. Pathological fibrosis is characterized by non-resolving or progressive tissue remodeling, which itself can cause tissue damage and organ failure.

Pathological fibroblasts are the drivers of many disease types, fibrosis being the salient feature. The primary hallmark of fibrosis is the chronic and excessive deposition of extracellular matrix by pathological fibroblasts and can arise in any organ of the body, ultimately resulting in organ failure. They can manifest after tissue insult or from idiopathic origin and become invasive, proliferative and deleteriously remodel organ tissue. The fibroblast populations in pathological setting arise from a mixed etiology of progenitor cells. Transdifferentiation of epidermal, endothelial, circulating bone marrow stem cells, pericytes and hepatic stellate cells all contribute to the total pool of fibroblasts in pathological setting depending on the effected organ. To date, there is no clear, clinical diagnostic marker to distinguish healthy, normal fibroblasts from pathological fibroblasts and no effective clinical treatment for the disease they cause such as fibrosis.

As such, there is an urgent need for diagnostic markers distinguishing normal fibroblasts from pathological fibroblasts, and related methods for treating conditions related to such pathological fibroblasts.

The present invention addresses such needs.

### SUMMARY OF THE INVENTION

Fibrosis is a major contributor to the chronic decline of organ function associated with end-stage organ failure. It is not unique to a single organ but manifests in any organ in the human body [Zeisberg, M.; Kalluri, R. Cellular Mechanisms of Tissue Fibrosis. 1. Common and organ-specific mechanisms associated with tissue fibrosis. Am. J. Physiol. Physiol. 2013, 304, C216-C225]. In the United States, forty-five percent of all deaths can be attributed to fibrosis-related disease [Wynn, T. A. Fibrotic disease and the TH1/TH2 paradigm. Nat. Rev. Immunol. 2004, 4, 583-594; Wynn, T. A. Cellular and molecular mechanisms of fibrosis. J. Pathol. 2008, 214, 199-210; Henderson, N. C.; Rieder, F.; Wynn, T. A. Fibrosis: From mechanisms to medicines. Nature 2020, 587, 555-566]. Fibrosis is described as the remodeling of tissue architecture which results in loss of organ function due to the substitution of the functional parenchyma with mesenchymal tissue. While transient tissue fibrosis is a normal response to injury during wound healing, pathological fibrosis is characterized by relentless, non-resolving extracellular matrix (ECM) deposition and progressive tissue remodeling. Fibrosis progresses when a poorly characterized wound healing process is activated, leading to excessive remodeling and deposition of ECM by myofibroblasts, primarily fibronectin, type I and type III collagen [Kubow, K. E.; Vukmirovic, R.; Zhe, L.; Klotzsch, E.; Smith, M. L.; Gourdon, D.; Luna, S.; Vogel, V. Mechanical forces regulate the interactions of fibronectin and collagen I in extracellular matrix. Nat. Commun. 2015, 6, 8026; Herrera, J.; Henke, C. A.; Bitterman, P. Extracellular matrix as a driver of progressive fibrosis. J. Clin. Investig. 2018, 128, 45-53]. Myofibroblasts are both ubiquitous and invasive as part of canonical wound healing; however, the origin of the myofibroblasts that fail to undergo programmed cell death or dedifferentiation following wound healing and result in progressive fibrosis remains ambiguous [Hinz, B.; Phan, S. H.; Thannickal, V. J.; Galli, A.; Bochaton-Piallat, M.-L.; Gabbiani, G. The Myofibroblast: One Function, Multiple Origins. Am. J. Pathol. 2007, 170, 1807-1816; Bagalad, B. S.; Kumar, K. P. M.; Puneeth, H. K. Myofibroblasts: Master of disguise. J. Oral Maxillofac. Pathol. 2017, 21, 462-463].

To date, there is no effective therapeutic to halt or reverse fibrotic progression and tractable therapeutic targets with pathological specificity are extremely limited [Kubow, K. E.; Vukmirovic, R.; Zhe, L.; Klotzsch, E.; Smith, M. L.; Gourdon, D.; Luna, S.; Vogel, V. Mechanical forces regulate the interactions of fibronectin and collagen I in extracellular matrix. Nat. Commun. 2015, 6, 8026; Caja, L.; Dituri, F.; Mancarella, S.; Caballero-Diaz, D.; Moustakas, A.; Giannelli, G.; Fabregat, I. TGF-β and the Tissue Microenvironment: Relevance in Fibrosis and Cancer. Int. J. Mol. Sci. 2018, 19, 1294; Wahl, J. K.; Kim, Y. J.; Cullen, J. M.; Johnson, K. R.; Wheelock, M. J. N-cadherin-Catenin Complexes Form Prior to Cleavage of the Proregion and Transport to the Plasma Membrane. J. Biol. Chem. 2003, 278, 17269-17276]. Only two therapeutics, Nintedanib and Pirfenidone, have been approved for fibrosis, and in only one organ system, the lungs. However, even with treatment idiopathic pulmonary fibrosis (IPF) continues to progress [Henderson, N. C.; Rieder, F.; Wynn, T. A. Fibrosis: From mechanisms to medicines. Nature 2020, 587, 555-566]. Understandably, there have been several attempts at therapeutic intervention designed to target proteins differentially expressed by myofibroblasts, including several successful attempts at abolishing fibrosis in rodent models [Caja, L.; Dituri, F.; Mancarella, S.; Caballero-Diaz, D.; Moustakas, A.; Giannelli, G.; Fabregat, I. TGF-β and the Tissue Microenvironment: Relevance in Fibrosis and Cancer. Int. J. Mol. Sci. 2018, 19, 1294; Biernacka, A.; Dobaczewski, M.; Frangogiannis, N. G. TGF-β signaling in fibrosis. Growth Factors 2011, 29, 196-202]. Notably, translating these models to the clinic has been challenging [Henderson, N. C.; Rieder, F.; Wynn, T. A. Fibrosis: From mechanisms to medicines. Nature 2020, 587, 555-566]. The salient example of this is the significant number of compounds targeting TGF-β1 and its associated signaling. TGF-β1 is a critical growth factor with roles in normal wound healing and immunity, as well as cancer progression, myofibroblast activity and fibrogenesis [Caja, L.; Dituri, F.; Mancarella, S.; Caballero-Diaz, D.; Moustakas, A.; Giannelli, G.; Fabregat, I. TGF-β and the Tissue Microenvironment: Relevance in Fibrosis and Cancer. Int. J. Mol. Sci. 2018, 19, 1294]. Neutralizing TGF-β1 signaling in rodent models abolishes fibrosis; however, molecules targeting TGF-β1 signaling in humans have failed to progress clinically [Biernacka, A.; Dobaczewski, M.; Frangogiannis, N. G. TGF-β signaling in fibrosis. Growth Factors 2011, 29, 196-202]. These failures to translate preclinical findings to the clinic represent an unmet need in the field to better identify therapeutic targets with specificity to fibrotic tissues.

Experiments conducted during the course of developing embodiments for the present invention demonstrated that retention of the N-cadherin prodomain at the cell surface is a potential biomarker of pathological myofibroblasts and fibrosis associated tissues of the heart, lungs, and liver. Canonical N-cadherin protein processing occurs in the transgolgi network after the precursor N-cadherin is translated with an N-terminal precursor prodomain, termed pro-N-cadherin (PNC) [Wahl, J. K.; Kim, Y. J.; Cullen, J. M.; Johnson, K. R.; Wheelock, M. J. N-cadherin-Catenin Complexes Form Prior to Cleavage of the Proregion and Transport to the Plasma Membrane. J. Biol. Chem. 2003, 278, 17269-17276; Koch, A. W.; Farooq, A.; Shan, W.; Zeng, L.; Colman, D. R.; Zhou, M.-M. Structure of the Neural (N—) Cadherin Prodomain Reveals a Cadherin Extracellular Domain-like Fold without Adhesive Characteristics. Structure 2004, 12, 793-805; Herrera, A.; Menendez, A.; Torroba, B.; Ochoa, A.; Pons, S. Dbnl and β-catenin promote pro-N-cadherin processing to maintain apico-basal polarity. J. Cell Biol. 2021, 220]. The precursor prodomain is then cleaved by proprotein convertases, generating the active mature N-cadherin protein which functions at the cell surface by forming homodimers at cellular adherens junctions [Wahl, J. K.; Kim, Y. J.; Cullen, J. M.; Johnson, K. R.; Wheelock, M. J. N-cadherin-Catenin Complexes Form Prior to Cleavage of the Proregion and Transport to the Plasma Membrane. J. Biol. Chem. 2003, 278, 17269-17276; Koch, A. W.; Farooq, A.; Shan, W.; Zeng, L.; Colman, D. R.; Zhou, M.-M. Structure of the Neural (N—) Cadherin Prodomain Reveals a Cadherin Extracellular Domain-like Fold without Adhesive Characteristics. Structure 2004, 12, 793-805]. The prodomain is non-adhesive and sterically restricts N-cadherin dimer formation by inhibiting N-cadherin tryptophan swapping [Koch, A. W.; Farooq, A.; Shan, W.; Zeng, L.; Colman, D. R.; Zhou, M.-M. Structure of the Neural (N—) Cadherin Prodomain Reveals a Cadherin Extracellular Domain-like Fold without Adhesive Characteristics. Structure 2004, 12, 793-805]. During certain developmental and pathological processes, PNC is localized to the cell surface with mature N-cadherin [Latefi, N. S.; Pedraza, L.; Schohl, A.; Li, Z.; Ruthazer, E. S. N-cadherin prodomain cleavage regulates synapse formationin vivo. Dev. Neurobiol. 2009, 69, 518-529; Maret, D.; Gruzglin, E.; Sadr, M. S.; Siu, V.; Shan, W.; Koch, A. W.; Seidah, N. G.; Del Maestro, R. F.; Colman, D. R. Surface Expression of Precursor N-cadherin Promotes Tumor Cell Invasion. Neoplasia 2010, 12, 1066-IN38; Nelson, E. R.; Li, S.; Kennedy, M.; Payne, S.; Kilibarda, K.; Groth, J.; Bowie, M.; Parilla-Castellar, E.; De Ridder, G.; Marcom, P. K.; et al. Chemotherapy enriches for an invasive triple-negative breast tumor cell subpopulation expressing a precursor form of N-cadherin on the cell surface. Oncotarget 2016, 7, 84030-84042]. To date, cell surface PNC expression has been observed in only two contexts: Perinatal synapse formation in vertebrates, and carcinogenesis [Latefi, N. S.; Pedraza, L.; Schohl, A.; Li, Z.; Ruthazer, E. S. N-cadherin prodomain cleavage regulates synapse formationin vivo. Dev. Neurobiol. 2009, 69, 518-529; Maret, D.; Gruzglin, E.; Sadr, M. S.; Siu, V.; Shan, W.; Koch, A. W.; Seidah, N. G.; Del Maestro, R. F.; Colman, D. R. Surface Expression of Precursor N-cadherin Promotes Tumor Cell Invasion. Neoplasia 2010, 12, 1066-IN38; Nelson, E. R.; Li, S.; Kennedy, M.; Payne, S.; Kilibarda, K.; Groth, J.; Bowie, M.; Parilla-Castellar, E.; De Ridder, G.; Marcom, P. K.; et al. Chemotherapy enriches for an invasive triple-negative breast tumor cell subpopulation expressing a precursor form of N-cadherin on the cell surface. Oncotarget 2016, 7, 84030-84042]. The experiments described herein describe a third clinically significant context in which PNC is expressed on the surface of myofibroblasts from pathological origins and aberrantly localized on tissues from failing heart, lung, and liver.

Heart failure is the leading cause of morbidity and mortality in the developed world and accounts for 1 in 8 deaths in the United States according to the Center for Disease Control (Heidenreich P A, Bozkurt B, Aguilar D, Allen L A, Byun J J, Colvin M M, Deswal A, Drazner M H, Dunlay S M, Evers L R, et al. 2022 AHA/ACC/HFSA Guideline for the Management of Heart Failure: A Report of the American College of Cardiology/American Heart Association Joint Committee on Clinical Practice Guidelines. Circulation. 2022; 145:e895-e1032). Despite this, the major molecular mechanisms of heart failure remain elusive, and treatment for heart failure is almost exclusively designed to alleviate symptoms after their onset. Even the diagnosis of heart failure suffers from a lack of consensus symptoms and biomarkers that define the onset of disease (Bozkurt B, Coats A J S, Tsutsui H, Abdelhamid C M, Adamopoulos S, Albert N, Anker S D, Atherton J, Bohm M, Butler J, et al. Universal definition and classification of heart failure: a report of the Heart Failure Society of America, Heart Failure Association of the European Society of Cardiology, Japanese Heart Failure Society and Writing Committee of the Universal Definition of Heart Failure: Endorsed by the Canadian Heart Failure Society, Heart Failure Association of India, Cardiac Society of Australia and New Zealand, and Chinese Heart Failure Association. Eur J Heart Fail. 2021; 23:352-380). This lack of understanding behind the molecular pathogenesis of heart failure has translated to a corresponding lack of molecular biomarkers that reflect early cardiac remodeling and accurately predict the development of heart failure prior to the onset of symptoms.

Current definitions of heart failure staging predict that approximately 50% of the general population over the age of 45 fall within stage A and B heart failure (Ammar K A, Jacobsen S J, Mahoney D W, Kors J A, Redfield M M, Burnett J C, Rodeheffer R J. Prevalence and Prognostic Significance of Heart Failure Stages. Circulation. 2007; 115:1563-1570). The standard of care serological biomarker for ruling in or ruling out heart failure is B-type Natriuretic Peptide (BNP) or it's precursor, N-terminal pro B-type Natriuretic Peptide (NTproBNP). NTpro/BNP functions as a natriuretic peptide that compensates for cardiac wall stress by inducing vasodilation leading to a reduction in cardiac filling pressure and increased cardiac output (Chen H H, Redfield M M, Nordstrom L J, Horton D P, Burnett J C. Subcutaneous administration of the cardiac hormone BNP in symptomatic human heart failure. Journal of Cardiac Failure. 2004; 10:115-119). This suggests that serum BNP levels are a surrogate for measuring cardiac wall stress, and increase after biochemical compensation pathways are triggered. Clinically, NTpro/BNP perform better for ruling out than ruling in heart failure with similar predictive value (Heidenreich P A, Bozkurt B, Aguilar D, Allen L A, Byun J J, Colvin M M, Deswal A, Drazner M H, Dunlay S M, Evers L R, et al. 2022 AHA/ACC/HFSA Guideline for the Management of Heart Failure: A Report of the American College of Cardiology/American Heart Association Joint Committee on Clinical Practice Guidelines. Circulation. 2022; 145:e895-e1032; McMurray J J, Adamopoulos S, Anker S D, Auricchio A, Böhm M, Dickstein K, Falk V, Filippatos G, Fonseca C, Gomez-Sanchez M A, et al. ESC Guidelines for the diagnosis and treatment of acute and chronic heart failure 2012: The Task Force for the Diagnosis and Treatment of Acute and Chronic Heart Failure 2012 of the European Society of Cardiology. Developed in collaboration with the Heart Failure Association (HFA) of the ESC. Eur Heart J. 2012; 33:1787-1847; Rørth R, Jhund P S, Yilmaz M B, Kristensen S L, Welsh P, Desai A S, Køber L, Prescott M F, Rouleau J L, Solomon S D, et al. Comparison of BNP and N T-proBNP in Patients With Heart Failure and Reduced Ejection Fraction. Circulation: Heart Failure. 2020; 13:e006541). In part this is due to a lack of consensus rule-in and rule-out standards. However, the analysis of serum NTpro/BNP must also be considered in the context of many other comorbidities and influential variables. These include age, sex, race, obesity and other cardiovascular and non-cardiovascular diseases and syndromes that can raise or lower NTpro/BNP in the blood (Bachmann K N, Gupta D K, Xu M, Brittain E, Farber-Eger E, Arora P, Collins S, Wells Q S, Wang T J. Unexpectedly Low Natriuretic Peptide Levels in Patients With Heart Failure. JACC: Heart Failure. 2021; 9:192-200; Parcha V, Patel N, Kalra R, Arora G, Januzzi J L, Felker G M, Wang T J, Arora P. Racial Differences in Serial N T-proBNP Levels in Heart Failure Management. Circulation. 2020; 142:1018-1020; Januzzi J L, Myhre P L. The Challenges of N T-proBNP Testing in HFpEF. JACC: Heart Failure. 2020; 8:382-385; Marie R, Philippe M. B-type natriuretic peptide and obesity in heart failure: a mysterious but important association in clinical practice. Cardiovascular Medicine. 2020; Mccullough P A, Kluger A Y. Interpreting the Wide Range of N T-proBNP Concentrations in Clinical Decision Making. J Am Coll Cardiol. 2018; 71:1201-1203; Myhre P L, Claggett B, Yu B, Skali H, Solomon S D, Røsjø H, Omland T, Wiggins K L, Psaty B M, Floyd J S, et al. Sex and Race Differences in N-Terminal Pro-B-type Natriuretic Peptide Concentration and Absolute Risk of Heart Failure in the Community. JAMA Cardiol. 2022; 7:623-631; Nishikimi T, Nakagawa Y. Potential pitfalls when interpreting plasma BNP levels in heart failure practice. J Cardiol. 2021; 78:269-274). Unfortunately, the earliest stage of heart failure in which NTpro/BNP values may be elevated is stage B (Heidenreich P A, Bozkurt B, Aguilar D, Allen L A, Byun J J, Colvin M M, Deswal A, Drazner M H, Dunlay S M, Evers L R, et al. 2022 AHA/ACC/HFSA Guideline for the Management of Heart Failure: A Report of the American College of Cardiology/American Heart Association Joint Committee on Clinical Practice Guidelines. Circulation. 2022; 145:e895-e1032; Ledwidge M, Gallagher J, Conlon C, Tallon E, O'Connell E, Dawkins I, Watson C, O'Hanlon R, Bermingham M, Patle A, et al. Natriuretic peptide-based screening and collaborative care for heart failure: the STOP-H F randomized trial. Jama. 2013; 310:66-74; Huelsmann M, Neuhold S, Resl M, Strunk G, Brath H, Francesconi C, Adlbrecht C, Prager R, Luger A, Pacher R, et al. PONTIAC (NT-proBNP selected prevention of cardiac events in a population of diabetic patients without a history of cardiac disease): a prospective randomized controlled trial. J Am Coll Cardiol. 2013; 62:1365-1372). In a study evaluating the prognostic value of NTproBNP for death and cardiovascular events in both normal and stage A/B heart failure subjects, the authors found that NTproBNP was not predictive of morbidity or mortality in healthy subjects (Nelson, E. R.; Li, S.; Kennedy, M.; Payne, S.; Kilibarda, K.; Groth, J.; Bowie, M.; Parilla-Castellar, E.; De Ridder, G.; Marcom, P. K.; et al. Chemotherapy enriches for an invasive triple-negative breast tumor cell subpopulation expressing a precursor form of N-cadherin on the cell surface. Oncotarget 2016, 7, 84030-84042). They further found that when comparing study participants with NTproBNP above the 80th percentile, the difference between those with stage A/B heart failure and healthy normal controls only amounted to 11.9% (24.7% vs 12.8%, respectively) (Nelson, E. R.; Li, S.; Kennedy, M.; Payne, S.; Kilibarda, K.; Groth, J.; Bowie, M.; Parilla-Castellar, E.; De Ridder, G.; Marcom, P. K.; et al. Chemotherapy enriches for an invasive triple-negative breast tumor cell subpopulation expressing a precursor form of N-cadherin on the cell surface. Oncotarget 2016, 7, 84030-84042). This “gray zone” of overlap between healthy normal individuals and those with stage A/B heart failure highlights a clear unmet need for an accurate and specific biomarker that can be used to screen and detect at-risk individuals.

In previous work, the inventors found that defective processing of N-cadherin in heart failure leads to cell-surface expression and aberrant localization of the precursor form of N-cadherin (pro-N-cadherin, PNC) on myofibroblasts and at intercalated discs in failing heart tissue (Schechter, M. A.; Hsieh, M. K. H.; Njoroge, L. W.; Thompson, J. W.; Soderblom, E. J.; Feger, B. J.; Troupes, C. D.; Hershberger, K.; Ilkayeva, O. R.; Nagel, W. L.; et al. Phosphoproteomic Profiling of Human Myocardial Tissues Distinguishes Ischemic from Non-Ischemic End Stage Heart Failure. PLOS ONE 2014, 9, e104157). With the prodomain intact, homophilic interactions found between N-cadherin in normal cellular junctions become sterically hindered, putatively disallowing the normal coordinated contractile functions of the cardiac muscle (Kostetskii, I.; Li, J.; Xiong, Y.; Zhou, R.; Ferrari, V. A.; Patel, V. V.; Molkentin, J. D.; Radice, G. L. Induced Deletion of the N-cadheringene in the Heart Leads to Dissolution of the Intercalated Disc Structure. Circ. Res. 2005, 96, 346-354). Consistent with our findings, Chen et al. recently reported a novel variant of N-cadherin identified in a 12-year-old female in which a point mutation resulted in retained prodomain at the cell surface (Chopra, A.; Tabdanov, E.; Patel, H.; Janmey, P. A.; Kresh, J. Y. Cardiac myocyte remodeling mediated by N-cadherin-dependent mechanosensing. Am. J. Physiol. Circ. Physiol. 2011, 300, H1252-H1266). The mutant N-cadherin had significantly impaired adhesion efficiency, and despite heterozygous expression of the mutation, the patient developed dilated cardiomyopathy and died of their disease at 13 years of age (Chopra, A.; Tabdanov, E.; Patel, H.; Janmey, P. A.; Kresh, J. Y. Cardiac myocyte remodeling mediated by N-cadherin-dependent mechanosensing. Am. J. Physiol. Circ. Physiol. 2011, 300, H1252-H1266). We further showed that the prodomain peptide can be detected in serum of patients with heart failure (Schechter, M. A.; Hsieh, M. K. H.; Njoroge, L. W.; Thompson, J. W.; Soderblom, E. J.; Feger, B. J.; Troupes, C. D.; Hershberger, K.; Ilkayeva, O. R.; Nagel, W. L.; et al. Phosphoproteomic Profiling of Human Myocardial Tissues Distinguishes Ischemic from Non-Ischemic End Stage Heart Failure. PLOS ONE 2014, 9, e104157). Experiments conducted herein examined the expression of soluble pro-N-cadherin as a biomarker for subclinical heart failure as compared to the standard marker NTproBNP.

Additional experiments described herein determined that pro-N-Cadherin is a marker of cardiac transplant rejection, a marker of organ transplant rejection, a predictor therapeutic response to an implanted medical device, and a marker of radiation induced fibrosis.

Accordingly, the present invention provides compositions and methods for detecting conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) in a subject through detecting aberrant pro-N-cadherin (PNC) localization and/or PNC circulation in blood (e.g., a blood, plasma, serum, a whole blood, buffy coat). In addition, the present invention provides methods for treating conditions characterized with pathological fibroblasts (e.g., heart failure) (e.g., early stages of heart failure) (e.g., cardiomyopathy) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) in a subject through inhibiting aberrant PNC cellular localization and/or PNC circulation in blood (e.g., a blood, plasma, serum, a whole blood, buffy coat).

One aspect of the present disclosure provides a method of detecting/diagnosing fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) in a subject comprising, consisting of, or consisting essentially of quantifying the amount of at least one biomarker present in a biological sample derived from the subject, wherein the biomarker is associated with fibrosis or conditions characterized with pathological fibroblasts. In some embodiments, the biomarker includes aberrant PNC cellular localization and/or PNC circulation in blood (e.g., a blood, plasma, serum, a whole blood, buffy coat).

Another aspect of the present disclosure provides a method of determining the presence of fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) in a subject comprising, consisting of, or consisting essentially of: (a) obtaining a biological sample from a subject; (b) determining the expression level of one or more biomarkers that are associated with fibrosis or pathological fibroblasts in the biological sample; (c) comparing the expression level of the biomarker(s) in the biological sample with that of a control, wherein the presence of one or more of the biomarker(s) in the sample that is in an amount greater than that of the control indicates fibrosis or a condition characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))); and (d) administering appropriate anti-fibrotic therapy if one or more of the biomarkers are expressed. In some embodiments, the biomarker includes aberrant PNC cellular localization and/or PNC circulation in blood (e.g., a blood, plasma, serum, a whole blood, buffy coat). In some embodiments, the sample is a selected from a blood sample, a plasma sample, a serum sample, a whole blood sample, and a buffy coat sample.

Another aspect of the present disclosure provides a method of diagnosing fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) in a subject comprising, consisting of, or consisting essentially of: (a) obtaining a biological sample from a subject; (b) determining the expression level of one or more biomarkers that are associated with fibrosis in the biological sample; (c) comparing the expression level of the biomarkers in the biological sample with that of a control, wherein the presence of one or more of the biomarkers in the sample that is in an amount greater than that of the control indicates fibrosis or conditions characterized with pathological fibroblasts; and (d) administering appropriate anti-fibrotic therapy if one or more of the biomarkers are expressed. In some embodiments, the biomarker includes aberrant PNC cellular localization and/or PNC circulation in blood (e.g., a blood, plasma, serum, a whole blood, buffy coat). In some embodiments, the sample is a selected from a blood sample, a plasma sample, a serum sample, a whole blood sample, and a buffy coat sample.

Another aspect of the present disclosure provides methods of prognosing or of aiding prognosis of fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) are provided. In such embodiments, a biological sample is obtained from the patient and the expression of aberrant PNC cellular localization and/or PNC circulation in blood measured. In certain embodiments, aberrant PNC cellular localization and/or PNC circulation is indicative of a prognosis for shortened survival compared to median survival. In such embodiments, reduced aberrant PNC cellular localization and/or PNC circulation is indicative of a prognosis for increased survival compared to median survival. In such embodiments, aberrant PNC cellular localization and/or PNC circulation is measured through use of an antibody specific for pro-N-cadherin.

Such methods are not limited to a particular anti-fibrotic therapy. In some embodiments, the anti-fibrotic therapy comprises any agent capable of inhibiting expression and/or activity related to pro-N-cadherin expression and/or activity. In some embodiments, the agent capable of inhibiting expression and/or activity related to pro-N-cadherin expression and/or activity is a small molecule, a polypeptide or peptide fragment, an siRNA, or an antibody or fragment thereof.

In some embodiments, the anti-fibrotic therapy includes compounds and compositions designed to stimulate the immune system to specifically recognize antigens expressed or overexpressed such pathological fibroblast cells (e.g., fibroblast cells expressing pro-N-cadherin). Non-limiting examples of antigen-specific immunotherapeutic agents include vaccines (e.g., peptide vaccines), antibodies, cytotoxic T cell lymphocytes (CTLs), chimeric antigen receptor T cells (CAR-T cells), and combinations thereof.

In other embodiments, the antibody comprises an antibody against pro-N-cadherin. In certain embodiments, the antibody comprises a monoclonal antibody (mAb). In some embodiments, the anti-pro-N-cadherin antibody is HC5LC4.

In some embodiments, the subject is a mammal. In other embodiments, the subject is a human.

In other embodiments, the biological sample is selected from the group consisting of tissues, cells, biopsies, blood, lymph, serum, plasma, urine, saliva, mucus, and tears. In certain embodiments, the sample comprises biopsies. In some embodiments, the sample is a selected from a blood sample, a plasma sample, a serum sample, a whole blood sample, and a buffy coat sample.

In some embodiments, the biological sample comprises fibroblast cells.

In some embodiments, the biological sample comprises lung tissue.

In some embodiments, the biological sample for use according to any of the above methods is lung tissue, whole blood, or serum. In some embodiments, the biological sample is lung tissue or whole blood and the expression of fibroblasts expressing pro-N-cadherin is measured using a PCR method or a microarray chip. In certain embodiments, the biological sample is serum and the expression of fibroblasts expressing pro-N-cadherin is measured using an immunoassay.

Such methods are not limited to a particular manner for measuring the biomarker associated with fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))). In some embodiments, the biomarker is measured through use of an antibody specific for fibroblasts expressing pro-N-cadherin. In some embodiments, the biomarker is measured by microarray. In another aspect gene expression of the biomarker (e.g., fibroblasts expressing pro-N-cadherin) is measured by real-time quantitative polymerase chain reaction (qPCR). In another aspect, gene expression is measured by multiplex-PCR. According to another embodiment, gene expression is measured by observing protein expression levels of fibroblasts expressing pro-N-cadherin. According to another embodiment, expression of pro-N-cadherin within fibroblast cells is considered elevated when compared to a healthy control or a reference subject if the relative mRNA level is greater than 2 fold of the level of a control or reference gene mRNA. According to another embodiment, the relative mRNA level is greater than 3 fold, fold, 10 fold, 15 fold, 20 fold, 25 fold, or 30 fold compared to a healthy control or reference gene expression level. In one aspect, the gene expression level is measured by a method selected from a PCR method, a microarray method, or an immunoassay method. In one embodiment, the microarray method comprises the use of a microarray chip having one or more nucleic acid molecules that can hybridize under stringent conditions to a nucleic acid molecule encoding fibroblasts expressing pro-N-cadherin or having one or more polypeptides (such as peptides or antibodies) that can bind to fibroblasts expressing pro-N-cadherin. In one embodiment, the PCR method is qPCR. In one embodiment, the PCR method is multiplex-PCR. According to one embodiment, the immunoassay method comprises binding an antibody to pro-N-cadherin expressed within fibroblast cells in a patient sample and determining if the level from the patient sample is elevated. In certain embodiments, the immunoassay method is an enzyme-linked immunosorbent assay (ELISA).

Such methods are not limited to a particular condition associated with pathological fibroblasts.

In some embodiments, the conditions characterized with pathological fibroblasts are selected from fibrotic liver disease, hepatic ischemia-reperfusion injury, cerebral infarction, ischemic heart disease, cardiac fibrosis, renal disease or lung (pulmonary) fibrosis. In other embodiments, the disease or condition is liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs, transplants or grafts; ischemia/reperfusion injury; stroke; cerebrovascular disease; myocardial ischemia; atherosclerosis; pancreatitis; renal failure; renal fibrosis; scleroderma; systemic sclerosis; dermal fibrosis and idiopathic pulmonary fibrosis. In still further embodiments, the treatment is for wounds for acceleration of healing; reducing post-surgical scarring; reducing adhesion formation; vascularization of a damaged and/or ischemic organ, transplant or graft; amelioration of ischemia/reperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; renal failure secondary to chronic diabetes and/or hypertension; muscular dystrophy, amyotrophic lateral sclerosis, fibrosis induced by chemical or environmental insult (e.g., cancer chemotherapy, pesticides, radiation/cancer radiotherapy); fibrosis induced by transplant (e.g., organ transplant) rejection; fibrosis induced by non-response of medically implanted device (e.g., left ventricular assist device (LVAD)); and/or diabetes mellitus.

In certain embodiments, subjects characterized as having fibrosis or conditions associated with pathological fibroblasts (e.g., fibroblast cells expressing pro-N-cadherin) are further administered one or more additional therapeutic agents. For example, in some embodiments, the additional therapeutic agent is selected from an anti-IL-13 agent, an anti-IL-4 agent, a combination anti-IL-13/anti-IL-4 agent, pirfenidone, anti-LOXL2 antibody (GS-6624), N-acetylcysteine, anti-TGF-.beta. antibody (GC1008), anti-.alpha.v.beta.6 integrin antibody (STX-100), anti-CTGF antibody (FG-3019), anti-CCL2 antibody (CNTO 888), somatostatin analog (SOM230, octreotide), antiotensin II inhibitor (losartan), carbon monoxide, thalidomide, tetrathiomolybdate, doxycycline, minocycline, and tyrosine kinase inhibitor (BIBF1120).

Yet another aspect of the present disclosure provides a method of treating and/or preventing fibrosis in a subject, the method comprising, consisting of, or consisting essentially of administering to the subject a therapeutically effective amount of a Pro-N-Cadherin inhibiting molecule such that the fibrosis is treated in the subject.

In some embodiments, the Pro-N-Cadherin inhibiting molecule is selected from the group consisting of a small molecule, an antibody, a miRNA, an antisense RNA, an oligonucleotide, aptamers, siRNA, peptides, polypeptides and combinations thereof.

In some embodiments, the inhibiting molecule comprises an antibody. In one embodiment, the antibody comprises a monoclonal antibody. In one embodiment, the antibody comprises 10A10.

The invention also provides kits comprising an antibody specific for fibroblasts expressing pro-N-cadherin and instructions for administering the compound to an animal. The kits may optionally contain other therapeutic agents, e.g., agents useful in treating fibrosis or conditions associated with pathological fibroblasts (e.g., fibroblasts expressing pro-N-cadherin).

### BRIEF DESCRIPTION OF THE DRAWINGS

FIG. 1A: Humanized α-PNC mAb variants ranked by dissociation rate. A total of 22 humanized PNC monoclonal antibody variants produced from the sequence of murine IgG1 clone 10A10. HC0 LC0 is an intermediate chimeric IgG4 mAb containing identical variable and CDR regions of murine 10A10. Variants highlighted in yellow did not fit well to a 1:1 binding model and were not pursued further.

FIG. 1B: Recombinant prodomain of N-cadherin was immobilized onto a high binding ELISA plate, blocked, then incubated with anti-his-tag antibody alone or with varying concentrations of anti-PNC antibody clone 10A10. Biotinylated anti-his-tag antibody was then detected using streptavidin-HRP.

FIG. 2A-B. PNC is aberrantly expressed in fibrotic heart, lung, and liver tissues. (A) Representative images of stained tissues from explanted failed human hearts (n=20, ischemic and non-ischemic etiology), lungs (n=10, IPF etiology) and livers (n=40, NAFLD-cirrhosis etiology) show positive expression and aberrant localization of PNC (Brown stain). Corresponding representative images of stained normal human heart (n=24), lung (n=18), and liver (n=32) show lack of aberrantly expressed PNC at the tissue level. Perinuclear staining, consistent with normal N-cadherin processing can be seen in the healthy cardiac tissue. Scale bar=100 μm. (B) Graphical representation of human samples analyzed in this study. Each column represents a single human sample, annotated by color to indicate the organ system of relevance to this study (top row), whether the patient had fibrosis (second row), the reported sex of the patient (third row), the age of the patient (fourth row) and the method by which the sample was analyzed in this study (fourth row). In cases where demographic data is unknown, unavailable, or unreported, a white bar with black hatch is shown. n=257 total samples analyzed.

FIG. 3A-C. Validation of myofibroblast phenotype of explant tissue derived cells and cell lines. (A) Rabbit IgG isotype control (blue shaded) was compared to α-SMA antibody (unshaded); Chi-squared=102.0; SE Dymax % Positive=32.1. (B) Rabbit IgG isotype control (blue shaded) was compared to type I collagen antibody (unshaded); Chi-squared=142.0; SE Dymax % Positive=36.0. (C) Total cell lysates were immunoblotted from each cell line to determine α-SMA protein expression and confirm myofibroblast phenotype.

FIG. 4A-B. PNC is localized to the cell surface of myofibroblasts. Myofibroblasts from heart and lung tissues were stained and analyzed by flow cytometry using m-α-PNC mAb 10A10, excluding debris and dead cells via gating and 7AAD exclusion using Flowjo software. Myofibroblasts from fibrosis origins stain positive for cell surface PNC: (A) CF-DCM, cardiac myofibroblasts from dilated cardiomyopathy; Chi-Squared=177.5; SE Dymax % Positive=48.0. LL97A, IPF; Chi-Squared=93.4; SE Dymax % Positive=54.8, and LL29, IPF; Chi-Squared=113.5; SE Dymax % Positive=51.0. PNC was not detected on the surface of primary normal human cardiac myofibroblasts from healthy donor, NHCF; Chi-Squared=3.47; SE Dymax % Positive=8.54, primary normal human lung myofibroblasts from healthy donor, NHLF; Chi-Squared=0; SE Dymax % Positive=0, or immortalized CCD-16Lu lung myofibroblasts from healthy donor; Chi-Squared=0; SE Dymax % Positive=0. Mouse IgG1 isotype control (blue shaded) was compared to m-α-PNC mAb (unshaded). Results are representative of 3 independent experiments, n=3. For all flow cytometry experiments, Chi-squared≥4 is statistically significant. (B) Cell surface proteins were isolated from myofibroblasts and immunoblotted for N-cadherin, PNC, and Na,k-ATPase α-1 (ATP1A1) cell surface compartment loading control. PNC and N-Cadherin lysates were normalized to the ATP1A1 cell surface loading control for each sample and are reported as a relative value below each band.

FIG. 5A-E. Validation of N-cadherin reagent specificity. CCD-16Lu were analyzed by flow cytometry following the flow cytometry protocol in the text to confirm no cross reactivity between α-PNC mAb and mature N-cadherin. (A) Analysis comparing mouse IgG1 isotype control (blue shaded) was compared to antibody to mature N-cadherin (unshaded); Chi-squared=301.4; SE Dymax % Positive=43.2. (B) Mouse IgG1 isotype control (blue shaded) was compared to antibody m-α-PNC mAb 10A10 (unshaded); Chi-squared=0; SE Dymax % Positive=10.9. (C) Human IgG4 isotype control (blue shaded) was compared to antibody h-α-PNC mAb HC5LC4 (unshaded); Chi-squared=1.2; SE Dymax % Positive=14.7. All antibodies were used at 5 μg/mL. (D) Total cell lysate from each cell line was immunoblotted for mature Ncadherin, PNC and RPL13A for a loading control. (E) Representative image of permeabilized and immunostained myofibroblasts with perinuclear PNC expression. Scale bars represent 25 μm.

FIG. 6. PNC is localized to cellular protrusions. Fixed, unpermeabilized myofibroblasts from fibrotic and healthy tissues were immunostained for m-α-PNC mAb 10A10 (red) and DAPI (blue). PNC is localized to cellular protrusions on pathological myofibroblasts DCM-CF, LL29, and LL97A (arrows). PNC is not expressed on the surface of NHCF, NHLF, or CCD-16Lu isolated from healthy donor.

FIG. 7A-F. Feasibility and development of a PNC ELISA. (A) Soluble PNC product was immunoprecipitated from LL29 conditioned media and patient plasma from pooled healthy donors, IPF patients, NAFLD-Cirrhosis patients and cardiomyopathy patients using m-α-PNC mAb 10A10 compared to mouse IgG1 isotype control (mIgG1) and immunoblotted. (B) Recombinant prodomain analyte was serially diluted in duplicate starting at 100 ng/ml in standard diluent and measured by ELISA to determine the range of the standard. (C) Linearity of endogenous analyte was measured. Plasma from patients with heart failure was serially diluted 1:3, 1:7, 1:15, 1:31 in standard diluent and sPNC was measured by ELISA in duplicate. All linear regression r² values were calculated to be within acceptable range (r²≥0.99). (D,E) Healthy donor plasma was diluted in standard diluent to indicated dilutions (1:1, 1:3, 1:7, 1:15) then spiked with recombinant prodomain analyte at 10 ng/ml (D) and 5 ng/ml (E) and analyzed by ELISA. Recovery of analyte was quantified and compared to back calculation of the standard. All dilutions were within the consensus range of 20 percent±the standard back calculations at concentrations 10 ng/mL and 5 ng/mL. (F) Plasma was assayed for sPNC from healthy controls (n=26), patients with IPF (n=9), NAFLD with cirrhosis (n=12), and cardiomyopathy (n=9). Ordinary one-way ANOVA analysis with Dunnett's multiple comparisons test was performed to determine significance (*p≤0.05, ****p≤0.0001).

FIG. 8A-C. FN1 is a potential PNC binding partner. (A) Medium binding ELISA plates were coated with human fibronectin, collagen type I, or collagen type III, then blocked and incubated with his-tagged recombinant N-cadherin prodomain. After washing unbound prodomain, prodomain binding to the immobilized substrate was measured using a biotinylated his-tag specific monoclonal antibody and streptavidin-HRP for detection. Assay was performed in quadruplicate and is representative of at least 3 independent experiments. (B) Representative image of a cardiac myofibroblast isolated from failed cardiac explant tissue showing colocalization of PNC and FN1 immunostained for PNC (red), FN1 (green) and DAPI (blue). Yellow indicates PNC/FN1 colocalization (Merge). (C) Medium binding ELISA plates were coated with fibronectin, blocked, then incubated with either recombinant prodomain of N-cadherin or prodomain in combination with mouse IgG1 isotype control, human IgG4 isotype control, m-α-PNC mAb 10A10 or h-α-PNC mAb HC5LC4. Bound recombinant prodomain was detected using anti-his-tag monoclonal antibody in technical duplicates and representative of at least 3 independent experiments (n=3). Ordinary one-way ANOVA analysis with Tukey's multiple comparisons test was performed to determine significance (*p≤0.05, **p≤0.01).

FIG. 9A-D. Cell surface PNC has a role in myofibroblast migration. Transwell permeable supports with a 6.5 mm polycarbonate membrane and 8 μm pores were coated with human fibronectin and used to separate the upper and lower chambers of a 24-well cell culture plate to measure migration of cells across the membrane (n=4). Pathological myofibroblast migration is significantly reduced by h-α-PNC mAb (HC5LC4) and recombinant prodomain of N-cadherin (rPro) after 5 h; (A) DCM-CF, dilated cardiomyopathy myofibroblasts (C) LL29, IPF myofibroblasts. No significant effect on myofibroblasts isolated from healthy tissues was observed; (D) immortalized CCD-16Lu lung myofibroblasts from healthy donor (B) NHCF, normal human cardiac myofibroblasts. Two tailed T-test assuming Gaussian distribution analysis was performed to determine significance. (*p≤0.05, **p≤0.01).

FIG. 10. AUROC analysis of plasma PNC for specificity and sensitivity. Normal donor samples (n=26) were compared to samples from cardiomyopathy (Red, n=9), NAFLD-Cirrhosis (Green, n=12) and IPF (Yellow, n=9) patients. To determine tissue-agnostic AUROC, all fibrotic samples were combined (Black, n=30) and compared to normal donor samples. AUROC=area under the receiver operating characteristics curve.

FIG. 11A-E. Relationship of pro-N-cadherin to NTproBNP and potential confounding variables—(A) Graphical representation of the percent distribution of NTproBNP rule-in, rule-out, and “gray zone”. (B) PNC values were analyzed between cohort A and cohort B by unpaired t test with Welch's corrections. Cohort B has significantly higher PNC levels with a mean value of 12.38 ng/mL relative to cohort A mean value of 7.37 ng/ml (p<0.0001, n=596). Red dots represent participants who meet the NTproBNP heart failure rule-in criteria, white/transparent dots fall within the NTproBNP rule-out criteria and gray represent participants who's NTproBNP falls within the “gray zone”. (C) Simple linear regression analysis of age versus PNC values for cohort A and cohort B. No correlation to age and PNC levels is observed in cohort A (slope 0.0132, r²=0.0002, slope non-zero p=0.79, n=289) but a slight correlation is found in cohort B (slope 0.1551, r²=0.0148, slope non-zero p=0.03, n=307). (D) Simple linear regression analysis of BMI versus PNC Values. The slope of either cohort A (slope 0.0298, r²=0.0003, slope non-zero p=0.78, n=289) nor cohort B (slope −0.0722, r²=0.0013, slope non-zero p=0.54, n=307) deviated significantly from 0 for BMI versus PNC values. (E) Correlation between PNC values and gender was analyzed using an Unpaired t test with Welch's correction. Neither cohort A (p=0.14, mean female 6.50 ng/mL, mean male 8.53 ng/mL, n=289) nor cohort B (p=0.21, mean female 11.29 ng/mL, mean male 13.79 ng/mL, n=307) differed in PNC values between females versus males.

FIG. 12. Graphical representation of the percent distribution of NTproBNP rule-in, rule-out, and “gray zone” of 1-5 yr follow-up (left) and 1-2 yr follow-up (right) subgroups.

FIG. 13. Pro-N-cadherin is a biomarker of subclinical heart failure. (Top) ROC analysis using Wilson/Brown method was performed comparing cohort A versus cohort B for participants who followed up within 1-2 years, 1-5 years, and 1-13 years. The AUC is greatest for participants who follow up within 1-2 years (p=. 0001, AUC 0.82 95% CI 0.71 to 0.93, total n=58), followed by 1-5 years (p<0.0001, AUC 0.72 95% CI 0.64 to 0.79, total n=168), and 1-13 years (p<0.001, AUC 0.66 95% CI 0.62 to 0.70, total n=596). (Bottom) ROC analysis using Wilson/Brown method of participants within cohort A excluding participants who meet the criteria for NTproBNP heart failure rule-in and participants who report CAD, heart attack, high blood pressure or atrial fibrillation at the time of blood draw versus cohort B participants who report at least 1, 2, or 3 heart failure risk factors. The AUC is greatest for participants with at least 3 heart failure risk factors (p<0.0001, AUC 0.81 95% CI 0.73 to 0.89 total n=150), followed by at least 2 risk factors (p<0.0001, AUC 0.76 95% CI 0.69 to 0.84 total n=188), and at least 1 risk factor (p<0.0001, AUC 0.74 95% CI 0.69 to 0.80 total n=342).

FIG. 14. PNC values correlate to NTproBNP values. (Top) Simple linear regression was used to determine a correlation between PNC levels and NTproBNP levels in each cohort. A modest correlation is observed in cohort A (slope 62.16, r²=0.56, slope non-zero p<0.0001, n=51) and a slight correlation is found in cohort B (slope 21.41, r²=0.10, slope non-zero p=0.0326, n=44). (Bottom) Inset of hashed area of top graphs.

FIG. 15A-G. Prognostic value of PNC levels demonstrated by survival curves. Comparison of survival curves was analyzed using log-rank test. (A) Cohort B has a significantly lower 13-year survival rate than cohort A (p=0.0016, logrank HR 1.64 95% CI 1.22 to 2.20, total n=596). (B) Participants from combined cohorts measuring PNC levels ≥6 ng/ml have significantly lower 13-year survival than participants measuring PNC levels <6 ng/ml (p<0.0001, logrank HR 1.99 95% CI 1.48 to 2.67, total n=596). (C) There is no significant difference between survival curves of cohort A between participants who PNC levels measure ≥6 ng/ml and participants that measure <6 ng/mL (p=0.5465, logrank HR 1.17 95% CI 0.69 to 1.98, total n=289). (D) Participants from cohort B measuring PNC levels ≥6 ng/ml have significantly lower 13-year survival than participants measuring PNC levels <6 ng/ml (p<0.0001, logrank HR 2.53 95% CI 1.74 to 3.69, total n=307). (E) No significant difference was found in 13-year survival between participants from combined cohorts measuring NTproBNP levels ≥300 pg/mL relative to participants measuring NTproBNP levels <300 pg/mL (p=0.2098, logrank HR 1.27 95% CI 0.84 to 1.92, total n=590). (F) No significant difference was found in 13-year survival between participants from cohort A measuring NTproBNP levels ≥300 pg/mL relative to participants measuring NTproBNP levels <300 pg/mL (p=0.0931, logrank HR 1.66 95% CI 0.82 to 3.33, total n=289). (G) No significant difference was found in 13-year survival between participants from cohort B measuring NTproBNP levels ≥300 pg/mL relative to participants measuring NTproBNP levels <300 pg/mL (p=0.8507, logrank HR 1.05 95% CI 0.63 to 1.75, total n=301). Each curve is depicted as the probability of survival and the 95% confidence interval.

FIG. 16A-C. Pro-N-Cadherin is a marker of cardiac transplant rejection. A—Post-operative human plasma samples were analyzed by previously described ELISA for PNC and compared to NTproBNP. PNC is significantly elevated in post cardiac transplant patients that progress to rejection stages 1R pAMR1, 2A or 3R compared to patients with no evidence of cardiac transplant rejection or 1R with mild rejection and no evidence of cell damage (p=0.0053). B—ROC curve representation of A (AUC=0.8519). C—No significant difference was observed in NTproBNP levels between the two cohorts (p=0.8078).

FIG. 17. PNC is a marker of rejection in a porcine model of cardiac transplant. All tissues were stained with anti-PNC antibody by IHC. As shown in FIG. 17, “R” prefix designated non-rejected porcine cardiac tissues and “D” prefix designated rejected porcine cardiac tissues. A, M, and P designated anterior, middle, and posterior, respectively, relative to the biopsy's location within the interventricular septum sample. Partially rejected tissue is outlined and labeled within FIG. 17. Stacked images within the same row represent different magnifications within the same sample.

FIG. 18. Pro-N-Cadherin is a predictor of a subset of left ventricular assist device (LVAD) non-responders. LVAD responders were defined by patients that maintained an increased ejection fraction by at least 10 percent post LVAD placement. Human plasma samples were analyzed by previously described ELISA for PNC and compared to NTproBNP. PNC was elevated in a subset of patients that do not respond to LVAD therapy (p=0.0536) (FIG. 18A). No significant difference was observed in NTproBNP levels between the two cohorts (p=0.8078) (FIG. 18B).

FIG. 19. Pro-N-cadherin is a marker of radiation induced fibrosis. FIG. 19 shows irradiated vs sham mouse colon stained with mouse-anti-mouse pro-N-cadherin antibody. Brown stain indicates cells positive for pro-N-cadherin protein. IR field consisted of approximately 2 cm of the rectum and distal colon starting from the anus (first 2 to 3 rings). FFPE tissues were stained using 10-20 μg/ml anti-pro-N-cadherin antibody.

FIG. 20 shows a graphical depiction of the experimental design evaluating the efficacy of anti-PNC monoclonal antibody therapy in doxorubicin-induced heart failure. Heart failure was induced by doxorubicin injection at Day 0, followed by a single antibody or IgG control administration 24 hours after initiation of doxorubicin treatment. A second doxorubicin injection was administered 7 days following initiation. Blood was collected at days 0 (pre-treatment), 1 (pre-treatment), 8 (post-treatment), and at termination.

FIG. 21 shows representative images of reduced aortic stenosis in doxorubicin induced heart failure guinea pigs followed by treatment with anti-pro-N-cadherin antibody HC5LC4. Blue stain is indicative of collagen fibers, a hallmark of cardiac remodeling.

FIG. 22 shows representative images of reduced cardiac myocyte hypertrophy and interstitial collagen in doxorubicin-induced heart failure followed by treatment with anti-pro-N-cadherin antibody HC5LC4 as compared to control in guinea pigs.

FIG. 23. NTproBNP was quantified in serum samples from guinea pigs at day 0, 1, 8 and 11. Percent change from baseline, day 0, was calculated for each animal and compared between treatment groups. NTproBNP is directly correlated with cardiac wall stress.

### DEFINITIONS

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

As used herein, the term “biomarker” refers to a naturally occurring biological molecule present in a subject at varying concentrations useful in predicting the risk or incidence of a disease or a condition, such as fibrosis. For example, the biomarker can be a protein present in higher or lower amounts in a subject at risk for fibrosis. The biomarker can include nucleic acids, ribonucleic acids, or a polypeptide used as an indicator or marker for fibrosis in the subject. In some embodiments, the biomarker comprises a protein. A biomarker may also comprise any naturally or non-naturally occurring polymorphism (e.g., single-nucleotide polymorphism [SNP]) present in a subject that is useful in predicting the risk or incidence of fibrosis. In certain embodiments, the biomarker comprises Pro-N-Cadherin. In certain embodiments, the biomarker comprises fibroblasts expressing pro-N-Cadherin.

As used herein, “fibrosis” refers to the formation of excess fibrous connective tissue as a result of the excess deposition of extracellular matrix components, for example collagen. Fibrous connective tissue is characterized by having extracellular matrix (ECM) with a high collagen content. The collagen may be provided in strands or fibers, which may be arranged irregularly or aligned. The ECM of fibrous connective tissue may also include glycosaminoglycans.

As used herein, “excess fibrous connective tissue” refers to an amount of connective tissue at a given location (e.g. a given tissue or organ, or part of a given tissue or organ) which is greater than the amount of connective tissue present at that location in the absence of fibrosis, e.g. under normal, non-pathological conditions. As used herein, “excess deposition of extracellular matrix components” refers to a level of deposition of one or more extracellular matrix components which is greater than the level of deposition in the absence of fibrosis, e.g. under normal, non-pathological conditions.

As used herein, the term “disease” refers to any abnormal condition and/or disorder of a structure or a function that affects a part of an organism. It may be caused by an external factor, such as an infectious disease, or by internal dysfunctions, such as fibrosis and the like.

As used herein, the term “fibrosis” also refers to those diseases/conditions associated with, or characterized by, fibrosis. Examples include, but are not limited to, respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma; chronic liver disease, primary biliary cirrhosis (PBC), schistosomal liver disease, liver cirrhosis; cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Chron's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease; diseases of the eye such as Grave's ophthalmopathy, epiretinal fibrosis, retinal fibrosis, subretinal fibrosis (e.g. associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis; arthritis; fibrotic pre-neoplastic and fibrotic neoplastic disease; and fibrosis induced by chemical or environmental insult (e.g., cancer chemotherapy, pesticides, radiation/cancer radiotherapy); fibrosis induced by transplant (e.g., organ transplant) rejection; and fibrosis induced by non-response of medically implanted device (e.g., left ventricular assist device (LVAD)).

### DETAILED DESCRIPTION OF THE INVENTION

The cellular and molecular mechanisms of fibrosis are described in Wynn, J. Pathol. (2008) 214(2): 199-210, and Wynn and Ramalingam, Nature Medicine (2012) 18:1028-1040, which are hereby incorporated by reference in their entirety.

The main cellular effectors of fibrosis are myofibroblasts, which produce a collagen-rich extracellular matrix.

In response to tissue injury, damaged cells and leukocytes produce pro-fibrotic factors such as TGF-β, IL-13 and PDGF, which activate fibroblasts to αSMA-expressing myofibroblasts, and recruit myofibroblasts to the site of injury. Myofibroblasts produce a large amount of extracellular matrix, and are important mediators in aiding contracture and closure of the wound. However, under conditions of persistent infection or during chronic inflammation there can be overactivation and recruitment of myofibroblasts, and thus over-production of extracellular matrix components, resulting in the formation of excess fibrous connective tissue.

In some embodiments, fibrosis may be triggered by pathological conditions, e.g. conditions, infections or disease states that lead to production of pro-fibrotic factors such as TGF-β1. In some embodiments, fibrosis may be caused by physical injury/stimuli, chemical injury/stimuli or environmental injury/stimuli. Physical injury/stimuli may occur during surgery, e.g. iatrogenic causes. Chemical injury/stimuli may include drug induced fibrosis, e.g. following chronic administration of drugs such as bleomycin, cyclophosphamide, amiodarone, procainamide, penicillamine, gold and nitrofurantoin (see, e.g., Daba et al., Saudi Med J 2004 June; 25(6): 700-6). Environmental injury/stimuli may include exposure to asbestos fibers or silica.

In embodiments herein, fibrosis may involve an organ of the gastrointestinal system, e.g. of the liver, small intestine, large intestine, or pancreas. In some embodiments, fibrosis may involve an organ of the respiratory system, e.g. the lungs. In embodiments, fibrosis may involve an organ of the cardiovascular system, e.g. of the heart or blood vessels. In some embodiments, fibrosis may involve the skin. In some embodiments, fibrosis may involve an organ of the nervous system, e.g. the brain. In some embodiments, fibrosis may involve an organ of the urinary system, e.g. the kidneys. In some embodiments, fibrosis may involve an organ of the musculoskeletal system, e.g. muscle tissue.

Fibrosis can occur in many tissues of the body. For example, fibrosis can occur in the liver (e.g. cirrhosis), lungs, kidney, heart, blood vessels, eye, skin, pancreas, intestine, brain, and bone marrow. Fibrosis may also occur in multiple organs at once.

Prior research has implicated the involvement of cell adhesion molecule N-cadherin in tissue fibrosis and remodeling. In experiments conducted during the course of developing embodiments for the present invention, it was hypothesized that anomalies in N-cadherin protein processing are involved in pathological fibrosis. Diseased tissues associated with fibrosis of the heart, lung, and liver were probed for the precursor form of N-cadherin, pro-N-cadherin (PNC), by immunohistochemistry and compared to healthy tissues. Myofibroblast cell lines were analyzed for cell surface pro-N-cadherin by flow cytometry and immunofluorescent microscopy. Soluble PNC products were immunoprecipitated from patient plasmas and an enzyme-linked immunoassay was developed for quantification. All fibrotic tissues examined show aberrant PNC localization. Cell surface PNC is expressed in myofibroblast cell lines isolated from cardiomyopathy and idiopathic pulmonary fibrosis but not on myofibroblasts isolated from healthy tissues. PNC is elevated in the plasma of patients with cardiomyopathy (p≤0.0001), idiopathic pulmonary fibrosis (p≤0.05), and nonalcoholic fatty liver disease with cirrhosis (p≤0.05). Finally, the inventors have humanized a murine antibody and demonstrate that it significantly inhibits migration of PNC expressing myofibroblasts. Collectively, the aberrant localization of PNC is observed in all fibrotic tissues examined in our study and the data suggest a role for cell surface PNC in the pathogenesis of fibrosis.

It was recently reported aberrant processing and localization of the precursor pro-N-cadherin (PNC) protein in failing heart tissues and detected elevated pro-N-cadherin products in the plasma of patients with heart failure. In experiments conducted during the course of developing embodiments for the present invention, it was hypothesized that PNC mislocalization and subsequent circulation is an early event in the pathogenesis of heart failure, and therefore circulating PNC is an early biomarker of heart failure. In collaboration with the Duke University Clinical and Translational Science Institute MURDOCK Study, such experiments queried enrolled individuals and sampled two matched cohorts: a cohort of individuals with no known heart failure at time of serum collection and no heart failure development in the following 13 years (n=289, cohort A) and a matching cohort of enrolled individuals who had no known heart failure at time of serum collection but subsequently developed heart failure within the following 13 years (n=307, cohort B). Serum PNC and N-terminal pro B-type natriuretic peptide (NTproBNP) concentrations in each population were quantified by ELISA. The experiments detected no significant difference in NTproBNP rule-in or rule-out statistics between the two cohorts at baseline. In participants that developed heart failure, serum PNC is significantly elevated relative to those that did not report development of heart failure (p<0.0001). Receiver-operating characteristic analyses of PNC demonstrate diagnostic value for subclinical heart failure. Additionally, PNC has diagnostic potential when comparing participants with no reported heart failure risk factors from cohort A to at-risk participants from cohort B over the 13-year follow up. Participants whose PNC levels measure greater than 6 ng/ml have a 41 percent increased risk of all-cause mortality independent of age, BMI, gender, NTproBNP, blood pressure, previous heart attack and coronary artery disease (p=0.044, n=596). These data indicate PNC is an early marker of heart failure and has the potential to identify patients who would benefit from early therapeutic intervention.

Additional experiments described herein determined that pro-N-Cadherin is a marker of cardiac transplant rejection, a marker of organ transplant rejection, a predictor therapeutic response to an implanted medical device, and a marker of radiation induced fibrosis.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

Accordingly, the present invention provides compositions and methods for detecting conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) in a subject through detecting aberrant pro-N-cadherin (PNC) cellular localization and/or PNC circulation in blood (e.g., a blood, plasma, serum, a whole blood, buffy coat). In addition, the present invention provides methods for treating conditions characterized with pathological fibroblasts (e.g., heart failure) (e.g., early stages of heart failure) (e.g., cardiomyopathy) (e.g., idiopathic pulmonary fibrosis) (e.g., nonalcoholic fatty liver disease with cirrhosis) in a subject through inhibiting aberrant PNC cellular localization and/or PNC circulation in blood (e.g., a blood, plasma, serum, a whole blood, buffy coat).

One aspect of the present disclosure provides a method of detecting/diagnosing fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) in a subject comprising, consisting of, or consisting essentially of quantifying the amount of at least one biomarker present in a biological sample derived from the subject, wherein the biomarker is associated with fibrosis or conditions characterized with pathological fibroblasts. In some embodiments, the biomarker includes aberrant PNC cellular localization and/or PNC circulation in blood (e.g., a blood, plasma, serum, a whole blood, buffy coat).

Another aspect of the present disclosure provides a method of determining the presence of fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) in a subject comprising, consisting of, or consisting essentially of: (a) obtaining a biological sample from a subject; (b) determining the expression level of one or more biomarkers that are associated with fibrosis or pathological fibroblasts in the biological sample; (c) comparing the expression level of the biomarker(s) in the biological sample with that of a control, wherein the presence of one or more of the biomarker(s) in the sample that is in an amount greater than that of the control indicates fibrosis or a condition characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))); and (d) administering appropriate anti-fibrotic therapy if one or more of the biomarkers are expressed. In some embodiments, the biomarker includes aberrant PNC cellular localization and/or PNC circulation in blood (e.g., a blood, plasma, serum, a whole blood, buffy coat). In some embodiments, the sample is a selected from a blood sample, a plasma sample, a serum sample, a whole blood sample, and a buffy coat sample.

Another aspect of the present disclosure provides a method of diagnosing fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) in a subject comprising, consisting of, or consisting essentially of: (a) obtaining a biological sample from a subject; (b) determining the expression level of one or more biomarkers that are associated with fibrosis in the biological sample; (c) comparing the expression level of the biomarkers in the biological sample with that of a control, wherein the presence of one or more of the biomarkers in the sample that is in an amount greater than that of the control indicates fibrosis or conditions characterized with pathological fibroblasts; and (d) administering appropriate anti-fibrotic therapy if one or more of the biomarkers are expressed. In some embodiments, the biomarker includes aberrant PNC cellular localization and/or PNC circulation in blood (e.g., a blood, plasma, serum, a whole blood, buffy coat). In some embodiments, the sample is a selected from a blood sample, a plasma sample, a serum sample, a whole blood sample, and a buffy coat sample.

Another aspect of the present disclosure provides methods of prognosing or of aiding prognosis of fibrosis or conditions characterized with pathological fibroblasts (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) are provided. In such embodiments, a biological sample is obtained from the patient and the expression of aberrant PNC cellular localization and/or PNC circulation in blood measured. In certain embodiments, aberrant PNC cellular localization and/or PNC circulation is indicative of a prognosis for shortened survival compared to median survival. In such embodiments, reduced aberrant PNC cellular localization and/or PNC circulation is indicative of a prognosis for increased survival compared to median survival. In such embodiments, aberrant PNC cellular localization and/or PNC circulation is measured through use of an antibody specific for pro-N-cadherin.

Such methods are not limited to a particular anti-fibrotic therapy. In some embodiments, the anti-fibrotic therapy comprises any agent capable of inhibiting expression and/or activity related to pro-N-cadherin expression and/or activity. In some embodiments, the agent capable of inhibiting expression and/or activity related to pro-N-cadherin expression and/or activity is a small molecule, a polypeptide or peptide fragment, an siRNA, or an antibody or fragment thereof.

In some embodiments, the anti-fibrotic therapy includes compounds and compositions designed to stimulate the immune system to specifically recognize antigens expressed or overexpressed such pathological fibroblast cells (e.g., fibroblast cells expressing pro-N-cadherin). Non-limiting examples of antigen-specific immunotherapeutic agents include vaccines (e.g., peptide vaccines), antibodies, cytotoxic T cell lymphocytes (CTLs), chimeric antigen receptor T cells (CAR-T cells), and combinations thereof.

In other embodiments, the antibody comprises an antibody against pro-N-cadherin. In certain embodiments, the antibody comprises a monoclonal antibody (mAb). In some embodiments, the anti-pro-N-cadherin antibody is HC5LC4.

Suitable pro-N-cadherin antibodies are antibodies that are able to bind specifically to pro-N-cadherin. In other words, they are antibodies that specifically bind to the N-terminal pro-N-cadherin region of N-cadherin that is cleaved to form N-cadherin. Suitable antibodies may bind the polypeptide sequence of the proregion of N-cadherein, (e.g. MCRIAGALRTLLPLLAALLQASVEASGEIALCKTGFPEDVYSAVLSKDVHEGQPLLNVK FSNCN GKRKVQYESSEPADFKVDEDGMVYAVRSFPLSSEHAKFLIYAQDKETQEKWQVAVKLS LKPTL TEESVKESAEVEEIVFPRQFSKHSGHLQRQKR (SEQ ID NO:1) taken from sequence of human N-cadherin, (GenBank™ accession NM_001792)). The antibody may bind an epitope of pro-N-cadherin that is linear or bind to a secondary structure formed in the pro-N-cadherin domain. The antibodies contemplated for use in the invention are antibodies specific to the pro-N-cadherin peptide (N-cadherin propeptide) and do not bind to the mature form of N-cadherin. As shown herein, pro-N-cadherin is specifically expressed on pathological fibroblast cells. As such, the use of an antibody specific to pro-N-cadherin allows for the specific targeting and killing of pathological fibroblast cells in contrast to an antibody that binds to the mature form of N-cadherin, which is found on other normal cell types (e.g. heart). Thus, the present invention, in one embodiment, provides a targeted therapy, e.g. antibody therapy against pro-N-cadherin that allows for the specific targeting of pathological fibroblast cells expressing pro-N-cadherin. One skilled in the art would be able to determine suitable antibodies for use in the present invention. Suitable examples include, but are not limited to, pro-N-cadherin antibody described in Wahl et al., (see, e.g., J Biol Chem. 2003; 278(19): 17269-17276; Maret D, et al., Neoplasia (New York, N.Y.). 2010; 12(12):1066-1080). Further a pro-N-cadherin antibody is commercially available from R&D systems (human N-cadherin propeptide antibody, available from Bio-Techne Corporation, Minneapolis, Minn.). The antibodies contemplated herein do not bind to the mature form of N-cadherin or non-pathological fibroblast cells.

The antibodies specific to pro-N-cadherin include whole antibodies (e.g., IgG, IgA, IgE, IgM, or IgD), monoclonal antibodies, polyclonal antibodies, and chimeric antibodies, humanized antibodies, and antibody fragments, including single chain variable fragments (ScFv), single domain antibody, and antigen-binding fragments, among others. In a preferred embodiment, the antibody is a monoclonal antibody.

In some embodiments, the anti-cancer drug/therapy is administered before the antibody. In other embodiments, the antibody is administered before the anti-cancer drug/therapy. In yet other embodiments, the antibody and anti-cancer drug/therapy are administered concurrently.

Pro-N-cadherin antibodies may be provided in combination with liposomes, nanoparticles or other analogous carriers loaded with an anti-cancer drug/therapy. Methods of preparing such compositions are known in the field (see, for example, Sugano et al., Antibody Targeting of Doxorubicin-loaded Liposomes Suppresses the Growth and Metastatic Spread of Established Human Lung Tumor Xenografts in Severe Combined Immunodeficient Mice, Cancer Research 60, 6942-6949, Dec. 15, 2000 and Martin et al., Nanomaterials in Analytical Chemistry, Analytical Chemistry News & Features, May 1, 1998; pp. 322 A-327 A).

In some embodiments, the subject is a mammal. In other embodiments, the subject is a human.

In other embodiments, the biological sample is selected from the group consisting of tissues, cells, biopsies, blood (e.g., a blood, plasma, serum, a whole blood, buffy coat), lymph, serum, plasma, urine, saliva, mucus, and tears. In certain embodiments, the sample comprises biopsies.

The present disclosure provides a method of determining the risk of, prognosis of, and/or diagnosis of a condition such as fibrosis or any condition associated with pathological fibroblast cells (e.g., heart failure (e.g., early stages of heart failure) (e.g., cardiomyopathy)) (e.g., idiopathic pulmonary fibrosis) (e.g., nonalcoholic fatty liver disease with cirrhosis) (e.g., fibrosis induced by chemical or environmental insult (e.g., cancer chemotherapy, pesticides, radiation/cancer radiotherapy)) (e.g., fibrosis induced by transplant (e.g., organ transplant) rejection) (e.g., fibrosis induced by non-response of medically implanted device (e.g., left ventricular assist device (LVAD))) on at least one sample obtained from a subject. In one embodiment, the subject is any mammal, but is preferably a human. The method comprises detecting and/or measuring the amount of at least one biomarker within the sample, wherein the biomarker is associated with the risk of, prognosis of, and/or diagnosis of the condition. In some embodiments, the biomarker includes aberrant PNC cellular localization and/or PNC circulation in blood (e.g., a blood, plasma, serum, a whole blood, buffy coat).

The present disclosure may involve obtaining more than one sample, such as two samples, such as three samples, four samples or more from subjects, and preferably the same subject. This allows the relative comparison of expression both in the presence or absence of at least biomarker (e.g. one nucleic acid) and/or the level of expression of the at least biomarker (e.g. one nucleic acid) between the two samples. Alternatively, a single sample may be compared against a “standardized” sample, such a sample comprising material or data from several samples, preferably also from several subjects.

Before analyzing the sample, it will often be desirable to perform one or more sample preparation operations upon the sample. Typically, these sample preparation operations will include such manipulations as concentration, suspension, extraction of intracellular material.

Any method required for the processing of a sample prior to detection by any of the methods noted herein falls within the scope of the present disclosure. These methods are typically well known by a person skilled in the art.

It is within the general scope of the present disclosure to provide methods for the detection of biomarkers (e.g., aberrant PNC cellular localization and/or PNC circulation in blood (e.g., a blood, plasma, serum, a whole blood, buffy coat)). An aspect of the present disclosure relates to the detection of the proteins as described in the plots and graphs of the figures contained herein. The present invention detects the protein of the pro-N-cadherin using a method that specifically detects the protein pro-N-cadherin and does not detect the processed or mature form of N-cadherin (e.g. the processed protein missing the pro-N-cadherin region). The present invention provides a method that specifically detects the cellular localization of PNC. The present invention provides a method that specifically detects the presence of PNC circulation in blood (e.g., a blood, plasma, serum, a whole blood, buffy coat).

As used herein, the term “detect” or “determine the presence of” refers to the qualitative measurement of undetectable, low, normal, or high concentrations of one or more biomarkers such as, for example, polypeptides of the pro-N-cadherin. Detection may include 1) detection in the sense of presence versus absence of one or more biomarkers as well as 2) the registration/quantification of the level or degree of expression of one or more biomarkers, depending on the method of detection employed. The term “quantify” or “quantification” may be used interchangeable, and refer to a process of determining the quantity or abundance of a substance in a sample (e.g., a biomarker), whether relative or absolute. For example, quantification may be determined by methods including but not limited to, any method able to detect proteins for example, immunohistochemistry, flow cytometry, band intensity on a Western blot, or by various other methods known in the art.

The detection of one or more biomarker molecules allows for the classification, diagnosis and prognosis of a condition such as fibrosis or any condition associated with pathological fibroblast cells (e.g., fibroblast cells expressing pro-N-cadherin). The classification of such conditions is of relevance both medically and scientifically and may provide important information useful for the diagnosis, prognosis and treatment of the condition. The diagnosis of a condition such as fibrosis or any condition associated with pathological fibroblast cells (e.g., fibroblast cells expressing pro-N-cadherin) is the affirmation of the presence of the condition, as is the object of the present disclosure, on the expression of at least one biomarker herein. Prognosis is the estimate or prediction of the probable outcome of a condition such as fibrosis and the prognosis of such is greatly facilitated by increasing the amount of information on the particular condition. The method of detection is thus a central aspect of the present disclosure.

Any method of detection falls within the general scope of the present disclosure. The detection methods may be generic for the detection of polypeptides and the like. The detection methods may be directed towards the scoring of a presence or absence of one or more biomarker molecules or may be useful in the detection of expression levels.

The detection methods can be divided into two categories herein referred to as in situ methods or screening methods. The term in situ method refers to the detection of protein molecules in a sample wherein the structure of the sample has been preserved. This may thus be a biopsy wherein the structure of the tissue is preserved. In situ methods are generally histological i.e. microscopic in nature and include but are not limited to methods such as: immunohistochemistry or any in situ methods able to detect proteins and polypeptides.

Screening methods generally employ techniques of molecular biology and most often require the preparation of the sample material in order to access the polypeptide molecules to be detected. Screening methods include, but are not limited to methods such as: flow cytometry, Western blot analysis, enzyme-linked immunosorbent assay (ELISA), and immunoelectrophoresis. Other methods understood and known by one skilled in the art for detecting proteins is contemplated for use in the present methods.

One aspect of the present disclosure is to provide a probe which can be used for the detection of a polypeptide molecule as defined herein. A probe as defined herein is a specific agent used to detect polypeptides by specifically binding to the protein, e.g. pro-N-cadherin. For example, an antibody or fragment thereof specific to pro-N-cadherin protein can be used as a probe to detect the biomarker, e.g. pro-N-cadherin in a sample. A probe may be labeled, tagged or immobilized or otherwise modified according to the requirements of the detection method chosen. A label or a tag is an entity making it possible to identify a compound to which it is associated. It is within the scope of the present disclosure to employ probes that are labeled or tagged by any means known in the art such as but not limited to: radioactive labeling, fluorescent labeling and enzymatic labeling. Furthermore the probe, labeled or not, may be immobilized to facilitate detection according to the detection method of choice and this may be accomplished according to the preferred method of the particular detection method.

The probes used may be to one or more biomarkers as disclosed herein. In a preferred embodiment, the probe is an antibody to pro-N-cadherin.

Another aspect of the present disclosure regards the detection of a biomarker which is a polypeptide molecules by any method known in the art. In the following are given examples of various detection methods that can be employed for this purpose, and the present disclosure includes all the mentioned methods, but is not limited to any of these.

Immunohistochemistry (IHC) involves the process of selectively imaging proteins in cells of a tissue section by using antibodies binding specifically to protein. Immunohistochemical staining is widely used in the diagnosis of abnormal cells such as those found in pathological cells (e.g., pathological fibroblast cells). Visualizing an antibody-antigen interaction can be accomplished in a number of ways known in the art, including, but not limited to, using an antibody conjugated to an enzyme, such as peroxidase, that can catalyze a color-producing reaction (e.g. immunoperoxidase staining), an antibody tagged or conjugated with a fluorophore, such as fluorescein or rhodamine (e.g. immunofluorescence), among others.

A probe used in IHC (e.g. an antibody or fragment thereof) can be labeled with a radioactive, fluorescent or antigenic tag, so that the probe's location and quantity in the tissue can be determined using autoradiography, fluorescence microscopy or immunoassay, respectively. The sample may be any sample as herein described. The probe is likewise a probe according to any probe based upon the biomarkers mentioned herein.

Flow cytometery can be used in the methods of detecting described herein. Flow cytometry is a laser- or impedance-based method that allows for cell counting, cell sorting, and biomarker detection by suspending cells in a stream of fluid and passing them through an electronic detection apparatus. The present methods include the use of flow cytometry to detect biomarkers on cells within samples taken from the subject. Suitable methods of flow cytometry are known in the art. In one suitable method, an antibody to pro-N-cadherin can be used in conjunction with a fluorescently tagged secondary antibody. In some embodiments, the pro-N-cadherin antibody may be directly conjugated to a fluorescence-tag. Methods of fluorescence-activated cell sorting (FACS) may also be used. It provides a method for sorting a heterogeneous mixture of biological cells into two or more containers, one cell at a time, based upon the specific light scattering and fluorescent characteristics of each cell.

Western blot (sometimes called the protein immunoblot) can be used in the detection methods described herein. Western blot methods are known in the art. For example, a sample may be separated by gel electrophoresis. Following electrophoretic separation, the proteins within the gel are transferred to a membrane (e.g., nitrocellulose or PVDF) on which the protein is then detected using a suitable probe, e.g. antibody specific to the biomarker. Using various methods such as staining, immunofluorescence, and radioactivity, visualization of the protein of interest can be detected on the membrane. Other suitable related techniques that can be used include, but are not limited to, dot blot analysis, and quantitative dot blot.

The enzyme-linked immunosorbent assay (ELISA) can also be used in the methods described herein. In some embodiments, the ELISA includes a solid-phase enzyme immunoassay (EIA) to detect the presence of a protein in a sample. ELISA also uses a probe, e.g. antibody specific to the biomarker to detect the biomarker within the sample. Suitable methods of performing ELISA are known in the art.

Immunoelectrophoresis can also be used in the methods described herein, for example, a number of biochemical methods for separation and characterization of proteins based on electrophoresis and reaction with antibodies are known in the art. The methods usually use antibodies specific to the protein to be detected.

In situ hybridization (ISH) applies and extrapolates the technology of nucleic acid and/or polypeptide hybridization to the single cell level, and, in combination with the art of cytochemistry, immunocytochemistry and immunohistochemistry, permits the maintenance of morphology and the identification of cellular markers to be maintained and identified, allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). DNA ISH can be used to determine the structure of chromosomes and the localization of individual genes and optionally their copy numbers. Fluorescent DNA ISH (FISH) can for example be used in medical diagnostics to assess chromosomal integrity. RNA ISH is used to assay expression and gene expression patterns in a tissue/across cells, such as the expression of miRNAs/nucleic acid molecules. Sample cells are treated to increase their permeability to allow the probe to enter the cells, the probe is added to the treated cells, allowed to hybridize at pertinent temperature, and then excess probe is washed away. A complementary probe is labeled with a radioactive, fluorescent or antigenic tag, so that the probe's location and quantity in the tissue can be determined using autoradiography, fluorescence microscopy or immunoassay, respectively. The sample may be any sample as herein described. The probe is likewise a probe according to any probe based upon the biomarkers mentioned herein.

In situ PCR is the PCR based amplification of the target nucleic acid sequences prior to ISH. For detection of RNA, an intracellular reverse transcription (RT) step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR the cells are cytocentrifugated onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens. Detection of intracellular PCR-products is achieved by one of two entirely different techniques. In indirect in situ PCR by ISH with PCR-product specific probes, or in direct in situ PCR without ISH through direct detection of labeled nucleotides (e.g. digoxigenin-11-dUTP, fluorescein-dUTP, 3H-CTP or biotin-16-dUTP) which have been incorporated into the PCR products during thermal cycling.

An embodiment of the present disclosure concerns the method of in situ PCR as mentioned herein above for the detection of nucleic acid molecules as detailed herein.

A microarray is a microscopic, ordered array of nucleic acids, proteins, small molecules, cells or other substances that enables parallel analysis of complex biochemical samples. A DNA microarray consists of different nucleic acid probes, known as capture probes that are chemically attached to a solid substrate, which can be a microchip, a glass slide or a microsphere-sized bead. Microarrays can be used e.g. to measure the expression levels of large numbers of polypeptides/proteins/nucleic acids simultaneously.

Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

An aspect of the present disclosure regards the use of microarrays for the expression profiling of biomarkers in conditions such as fibrosis or conditions associated with pathological fibroblast cells. For this purpose, and by way of example, RNA is extracted from a cell or tissue sample, the small RNAs (18-26-nucleotide RNAs) are size-selected from total RNA using denaturing polyacrylamide gel electrophoresis (PAGE). Then oligonucleotide linkers are attached to the 5′ and 3′ ends of the small RNAs and the resulting ligation products are used as templates for an RT-PCR reaction with 10 cycles of amplification. The sense strand PCR primer has a Cy3 fluorophore attached to its 5′ end, thereby fluorescently labelling the sense strand of the PCR product. The PCR product is denatured and then hybridized to the microarray. A PCR product, referred to as the target nucleic acid that is complementary to the corresponding RNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular biomarker, using a number of positive and negative controls and array data normalization methods, which will result in assessment of the level of expression of a particular biomarker.

Several types of microarrays can be employed such as spotted oligonucleotide microarrays, pre-fabricated oligonucleotide microarrays or spotted long oligonucleotide arrays.

In spotted oligonucleotide microarrays the capture probes are oligonucleotides complementary to nucleic acid sequences. This type of array is typically hybridized with amplified.

PCR products of size-selected small RNAs from two samples to be compared that are labelled with two different fluorophores. Alternatively, total RNA containing the small RNA fraction is extracted from the abovementioned two samples and used directly without size-selection of small RNAs, and 3′ end labeled using T4 RNA ligase and short RNA linkers labelled with two different fluorophores. The samples can be mixed and hybridized to one single microarray that is then scanned, allowing the visualization of up-regulated and down-regulated biomarker genes in one go. The downside of this is that the absolute levels of gene expression cannot be observed, but the cost of the experiment is reduced by half. Alternatively, a universal reference can be used, comprising of a large set of fluorophore-labelled oligonucleotides, complementary to the array capture probes.

In pre-fabricated oligonucleotide microarrays or single-channel microarrays, the probes are designed to match the sequences of known or predicted biomarkers. There are commercially available designs that cover complete genomes from companies such as Affymetrix, or Agilent. These microarrays give estimations of the absolute value of gene expression and therefore the comparison of two conditions requires the use of two separate microarrays.

Spotted long oligonucleotide arrays are composed of 50 to 70-mer oligonucleotide capture probes, and are produced by either ink-jet or robotic printing. Short Oligonucleotide Arrays are composed of 20-25-mer oligonucleotide probes, and are produced by photolithographic synthesis (Affymetrix) or by robotic printing. More recently, Maskless Array Synthesis from NimbleGen Systems has combined flexibility with large numbers of probes. Arrays can contain up to 390,000 spots, from a custom array design.

The terms “PCR reaction”, “PCR amplification”, “PCR”, “pre-PCR”, “Q-PCR”, “real-time quantitative PCR” and “real-time quantitative RT-PCR” are interchangeable terms used to signify use of a nucleic acid amplification system, which multiplies the target nucleic acids being detected. Examples of such systems include the polymerase chain reaction (PCR) system and the ligase chain reaction (LCR) system. Other methods recently described and known to the person of skill in the art are the nucleic acid sequence-based amplification and Q Beta Replicase systems. The products formed by said amplification reaction may or may not be monitored in real time or only after the reaction as an end-point measurement.

Real-time quantitative RT-PCR is a modification of polymerase chain reaction used to rapidly measure the quantity of a product of polymerase chain reaction. It is preferably done in real-time, thus it is an indirect method for quantitatively measuring starting amounts of DNA, complementary DNA or ribonucleic acid (RNA). This is commonly used for the purpose of determining whether a genetic sequence is present or not, and if it is present the number of copies in the sample. There are 3 methods which vary in difficulty and detail. Like other forms of polymerase chain reaction, the process is used to amplify DNA samples, using thermal cycling and a thermostable DNA polymerase.

The three commonly used methods of quantitative polymerase chain reaction are through agarose gel electrophoresis, the use of SYBR Green, a double stranded DNA dye, and the fluorescent reporter probe. The latter two of these three can be analyzed in real-time, constituting real-time polymerase chain reaction method.

Agarose gel electrophoresis is the simplest method, but also often slow and less accurate then other methods, depending on the running of an agarose gel via electrophoresis. It cannot give results in real time. The unknown sample and a known sample are prepared with a known concentration of a similarly sized section of target DNA for amplification. Both reactions are run for the same length of time in identical conditions (preferably using the same primers, or at least primers of similar annealing temperatures). Agarose gel electrophoresis is used to separate the products of the reaction from their original DNA and spare primers. The relative quantities of the known and unknown samples are measured to determine the quantity of the unknown. This method is generally used as a simple measure of whether the probe target sequences are present or not, and rarely as ‘true’ Q-PCR.

Using SYBR Green dye is more accurate than the gel method, and gives results in real time. A DNA binding dye binds all newly synthesized double stranded (ds)DNA and an increase in fluorescence intensity is measured, thus allowing initial concentrations to be determined. However, SYBR Green will label all dsDNA including any unexpected PCR products as well as primer dimers, leading to potential complications and artefacts. The reaction is prepared as usual, with the addition of fluorescent dsDNA dye. The reaction is run, and the levels of fluorescence are monitored; the dye only fluoresces when bound to the dsDNA. With reference to a standard sample or a standard curve, the dsDNA concentration in the PCR can be determined.

The fluorescent reporter probe method is the most accurate and most reliable of the methods. It uses a sequence-specific nucleic acid-based probe so as to only quantify the probe sequence and not all double stranded DNA. It is commonly carried out with DNA based probes with a fluorescent reporter and a quencher held in adjacent positions, so-called dual-labelled probes. The close proximity of the reporter to the quencher prevents its fluorescence; it is only on the breakdown of the probe that the fluorescence is detected. This process depends on the 5′ to 3′ exonuclease activity of the polymerase involved. The real-time quantitative PCR reaction is prepared with the addition of the dual-labelled probe. On denaturation of the double-stranded DNA template, the probe is able to bind to its complementary sequence in the region of interest of the template DNA (as the primers will too). When the PCR reaction mixture is heated to activate the polymerase, the polymerase starts synthesizing the complementary strand to the primed single stranded template DNA. As the polymerization continues it reaches the probe bound to its complementary sequence, which is then hydrolyzed due to the 5′-3′ exonuclease activity of the polymerase thereby separating the fluorescent reporter and the quencher molecules. This results in an increase in fluorescence, which is detected. During thermal cycling of the real-time PCR reaction, the increase in fluorescence, as released from the hydrolyzed dual-labelled probe in each PCR cycle is monitored, which allows accurate determination of the final, and so initial, quantities of DNA.

Any method of PCR that can determine the expression of a nucleic acid molecule as defined herein falls within the scope of the present disclosure. A preferred embodiment of the present disclosure includes the real-time quantitative RT-PCR method, based on the use of either SYBR Green dye or a dual-labelled probe for the detection and quantification of nucleic acids according to the herein described.

Yet another aspect of the present disclosure provides a method of treating and/or preventing fibrosis in a subject, the method comprising, consisting of, or consisting essentially of administering to the subject a therapeutically effective amount of a Pro-N-Cadherin inhibiting molecule such that the fibrosis is treated in the subject.

The targeting of Pro-N-Cadherin offers multiple routes for inhibiting its expression and/or function. Such inhibitory molecules may prevent the expression of Pro-N-Cadherin and/or inhibit the function of Pro-N-Cadherin and/or induce the cleaving of Pro-N-Cadherin to its mature form, N-Cadherin. Suitable inhibitory molecules include, but are not limited to, small molecules, antibodies, miRNAs, antisense RNAs, oligonucleotides, aptamers, siRNAs, peptides, polypeptides, and combinations thereof.

In this specification “antibody” includes a fragment or derivative of an antibody, or a synthetic antibody or synthetic antibody fragment.

In some embodiments, the anti-pro-N-cadherin antibody is HC5LC4.

Suitable anti-Pro-N-Cadherin antibodies will preferably bind to Pro-N-Cadherin (the antigen), preferably human Pro-N-Cadherin, and may have a dissociation constant (K_{D}) of one of 1 μM, 100 μM, 10 μM, 1 nM or 100 pM. Binding affinity of an antibody for its target is often described in terms of its dissociation constant (K_{D}). Binding affinity can be measured by methods known in the art, such as by Surface Plasmon Resonance (SPR), or by a radiolabeled antigen binding assay (RIA) performed with the Fab version of the antibody and antigen molecule.

Anti-Pro-N-Cadherin antibodies may be antagonist antibodies that inhibit or reduce a biological activity of Pro-N-Cadherin. Anti-Pro-N-Cadherin antibodies may be antagonist antibodies that inhibit or reduce any function of Pro-N-Cadherin, in particular signaling.

Anti-Pro-N-Cadherin antibodies may be neutralizing antibodies that neutralize the biological effect of Pro-N-Cadherin, e.g. its ability to initiate productive signaling mediated by binding of Pro-N-Cadherin.

In some embodiments, the antibody is a polyclonal antibody. In other embodiments, the antibody is a monoclonal. An example of a known anti-Pro-N-Cadherin monoclonal antibody comprises 10A10. In certain embodiments, monoclonal antibody is a humanized monoclonal antibody.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799).

Monoclonal antibodies (mAbs) are useful in the methods of the disclosure and are a homogenous population of antibodies specifically targeting a single epitope on an antigen.

Polyclonal antibodies are useful in the methods of the disclosure. Monospecific polyclonal antibodies are preferred. Suitable polyclonal antibodies can be prepared using methods well known in the art.

Antigen binding fragments of antibodies, such as Fab and Fab₂ fragments may also be used/provided as can genetically engineered antibodies and antibody fragments. The variable heavy (V_{H}) and variable light (V_{L}) domains of the antibody are involved in antigen recognition, a fact first recognized by early protease digestion experiments. Further confirmation was found by “humanization” of rodent antibodies. Variable domains of rodent origin may be fused to constant domains of human origin such that the resultant antibody retains the antigenic specificity of the rodent parented antibody (see, e.g., Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (see, e.g., Better et al (1988) Science 240, 1041); Fv molecules (see, e.g., Skerra et al (1988) Science 240, 1038); single-chain Fv (ScFv) molecules where the V_{H} and V_{L} partner domains are linked via a flexible oligopeptide (see, e.g., Bird et al (1988) Science 242, 423; Huston et al (1988) Proc. Natl. Acad. Sd. USA 85, 5879) and single domain antibodies (dAbs) comprising isolated V domains (see, e.g., Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are covalently linked, e.g. by a flexible oligopeptide.

Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining site. Synthetic antibodies which bind to Pro-N-Cadherin may also be made using phage display technology as is well known in the art.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):331 0-15 9 (1995); and Hawkins et al, J. Mol. Biol. 226:889-896 (1992).

Antibodies according to the present disclosure preferably exhibit specific binding to Pro-N-Cadherin. An antibody that specifically binds to a target molecule preferably binds the target with greater affinity, and/or with greater duration than it binds to other targets. In one embodiment, the extent of binding of an antibody to an unrelated target is less than about 10% of the binding of the antibody to the target as measured, e.g., by ELISA, or by a radioimmunoassay (RIA). Alternatively, the binding specificity may be reflected in terms of binding affinity where the antibody binds to Pro-N-Cadherin with a K_{D} that is at least 0.1 order of magnitude (i.e. 0.1×10n, where n is an integer representing the order of magnitude) greater than the K_{D} of the antibody towards another target molecule.

Antibodies may be detectably labelled or, at least, capable of detection. Such antibodies being useful for both in vivo (e.g. imaging methods) and in vitro (e.g. assay methods) applications. For example, the antibody may be labelled with a radioactive atom or a colored molecule or a fluorescent molecule or a molecule which can be readily detected in any other way. Suitable detectable molecules include fluorescent proteins, luciferase, enzyme substrates, and radiolabels. The binding moiety may be directly labelled with a detectable label or it may be indirectly labelled. For example, the binding moiety may be an unlabeled antibody which can be detected by another antibody which is itself labelled. Alternatively, the second antibody may have bound to it biotin and binding of labelled streptavidin to the biotin is used to indirectly label the first antibody.

Aspects of the present disclosure include bi-specific antibodies, e.g. composed of two different fragments of two different antibodies, such that the bi-specific antibody binds two types of antigen. One of the antigens is Pro-N-Cadherin, the bi-specific antibody comprising a fragment as described herein that binds to Pro-N-Cadherin. The antibody may contain a different fragment having affinity for a second antigen, which may be any desired antigen. Techniques for the preparation of bi-specific antibodies are well known in the art, e.g. see Mueller, D et al., (2010 Biodrugs 24 (2): 89-98), Wozniak-Knopp G et al., (2010 Protein Eng Des 23 (4): 289-297. Baeuerle, P A et al., (2009 Cancer Res 69 (12): 4941-4944).

In some embodiments, the bispecific antibody is provided as a fusion protein of two single-chain variable fragments (scFV) format, comprising a V_{H} and V_{L} of a Pro-N-Cadherin binding antibody or antibody fragment, and a V_{H} and V_{L} of an another antibody or antibody fragment.

Bispecific antibodies and bispecific antigen binding fragments may be provided in any suitable format, such as those formats described in Kontermann MAbs 2012, 4(2): 182-197, which is hereby incorporated by reference in its entirety.

Methods for producing bispecific antibodies include chemically crosslinking antibodies or antibody fragments, e.g. with reducible disulfide or non-reducible thioether bonds, for example as described in Segal and Bast, 2001. Production of Bispecific Antibodies. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH— groups, to create disulfide-linked bispecific F(ab)₂ heterodimers.

Other methods for producing bispecific antibodies include fusing antibody-producing hybridomas e.g. with polyethylene glycol, to produce a quadroma cell capable of secreting bispecific antibody, for example as described in D. M. and Bast, B. J. 2001. Production of Bispecific Antibodies. Current Protocols in Immunology. 14:IV:2.13:2.13.1-2.13.16.

Bispecific antibodies and bispecific antigen binding fragments can also be produced recombinantly, by expression from e.g. a nucleic acid construct encoding polypeptides for the antigen binding molecules, for example as described in Antibody Engineering: Methods and Protocols, Second Edition (Humana Press, 2012), at Chapter 40: Production of Bispecific Antibodies: Diabodies and Tandem scFv (Hornig and Farber-Schwarz), or French, How to make bispecific antibodies, Methods Mol. Med. 2000; 40:333-339.

For example, a DNA construct encoding the light and heavy chain variable domains for the two antigen binding domains (i.e. the light and heavy chain variable domains for the antigen binding domain capable of binding Pro-N-Cadherin, and the light and heavy chain variable domains for the antigen binding domain capable of binding to another target protein), and including sequences encoding a suitable linker or dimerization domain between the antigen binding domains can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

Peptide or polypeptide based Pro-N-Cadherin binding agents may be based on the Pro-N-Cadherin protein or a fragment of Pro-N-Cadherin.

In other embodiments a Pro-N-Cadherin inhibiting molecule may be provided in the form of a small molecule inhibitor of Pro-N-Cadherin.

Peptide or polypeptide based Pro-N-Cadherin inhibitory molecules may be based on Pro-N-Cadherin, e.g. mutant, variant or binding fragment of Pro-N-Cadherin. Suitable peptide or polypeptide-based molecules may bind to Pro-N-Cadherin in a manner that does not lead to initiation of signal transduction or produces sub-optimal signaling.

In some embodiments a pro-N-Cadherin inhibitory molecule may be provided in the form of a small molecule inhibitor of pro-N-Cadherin.

In some aspects of the present disclosure treatment, prevention or alleviation of fibrosis may be provided by administration of an inhibitory molecule capable of preventing or reducing the expression of Pro-N-Cadherin by cells of the subject, e.g. by fibroblasts or myofibroblasts.

In some embodiments an inhibitory molecule capable of preventing or reducing the expression of Pro-N-Cadherin may be an oligonucleotide capable of repressing or silencing expression of Pro-N-Cadherin.

Accordingly, the present disclosure also includes the use of techniques known in the art for the therapeutic down regulation of Pro-N-Cadherin expression. These include the use of antisense oligonucleotides and RNA interference (RNAi). As in other aspects of the present disclosure, these techniques may be used in the treatment of fibrosis.

Accordingly, in one aspect of the present disclosure a method of treating or preventing fibrosis is provided, the method comprising, consisting of, or consisting essentially of administering to a subject in need of treatment a therapeutically effective amount of Pro-N-Cadherin inhibiting molecule capable of preventing or reducing the expression of Pro-N-Cadherin, wherein the molecule comprises a vector comprising a therapeutic oligonucleotide capable of repressing or silencing expression of Pro-N-Cadherin.

In another aspect of the present invention a method of treating or preventing fibrosis is provided, the method comprising, consisting of, or consisting essentially of administering to a subject in need of treatment a therapeutically effective amount of an agent capable of preventing or reducing the expression of Pro-N-Cadherin, wherein the agent comprises an oligonucleotide vector, optionally a viral vector, encoding a therapeutic oligonucleotide capable of being expressed in cells of the subject, the expressed therapeutic oligonucleotide being capable of repressing or silencing expression of Pro-N-Cadherin.

The ability of an inhibiting molecule to prevent or reduce the expression of Pro-N-Cadherin may be assayed by determining the ability of the agent to inhibit Pro-N-Cadherin gene or protein expression by fibroblasts or myofibroblasts.

Aptamers, also called nucleic acid ligands, are nucleic acid molecules characterized by the ability to bind to a target molecule with high specificity and high affinity. Almost every aptamer identified to date is a non-naturally occurring molecule.

Aptamers to a given target (e.g. Pro-N-Cadherin) may be identified and/or produced by the method of Systematic Evolution of Ligands by EXponential enrichment (SELEX™). Aptamers and SELEX are described in Tuerk and Gold (Systematic evolution of ligands by exponential enrichment: RNA ligands to bacteriophage T4 DNA polymerase. Science. 1990 Aug. 3; 249(4968):505-10) and in WO91/19813.

Aptamers may be DNA or RNA molecules and may be single stranded or double stranded. The aptamer may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose.

Aptamers may be synthesized by methods which are well known to the skilled person. For example, aptamers may be chemically synthesized, e.g. on a solid support.

Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer.

Aptamers can be thought of as the nucleic acid equivalent of monoclonal antibodies and often have K_{D}'s in the nM or pM range, e.g. less than one of 500 nM, 100 nM, 50 nM, 10 nM, 1 nM, 500 pM, 100 pM. As with monoclonal antibodies, they may be useful in virtually any situation in which target binding is required, including use in therapeutic and diagnostic applications, in vitro or in vivo. In vitro diagnostic applications may include use in detecting the presence or absence of a target molecule.

Aptamers according to the present disclosure may be provided in purified or isolated form. Aptamers according to the present disclosure may be formulated as a pharmaceutical composition or medicament.

Suitable aptamers may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

Suitable aptamers may optionally have a maximum length of one of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

Suitable aptamers may optionally have a length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

Oligonucleotide molecules, particularly RNA, may be employed to regulate gene expression. These include antisense oligonucleotides, targeted degradation of mRNAs by small interfering RNAs (siRNAs), small molecules, post transcriptional gene silencing (PTGs), developmentally regulated sequence-specific translational repression of mRNA by micro-RNAs (miRNAs) and targeted transcriptional gene silencing.

An antisense oligonucleotide is an oligonucleotide, preferably single stranded, that targets and binds, by complementary sequence binding, to a target oligonucleotide, e.g. mRNA. Where the target oligonucleotide is an mRNA, binding of the antisense to the mRNA blocks translation of the mRNA and expression of the gene product. Antisense oligonucleotides may be designed to bind sense genomic nucleic acid and inhibit transcription of a target nucleotide sequence.

In view of the known nucleic acid sequences for Pro-N-Cadherin, oligonucleotides may be designed to repress or silence the expression of Pro-N-Cadherin. Such oligonucleotides may have any length, but may preferably be short, e.g. less than 100 nucleotides, e.g. 10-40 nucleotides, or 20-50 nucleotides, and may comprise a nucleotide sequence having complete- or near-complementarity (e.g. 80%, 85%, 90%, 91%, 92%, 93%, 94%, 95%, 96%, 97%, 98%, 99% or 100% complementarity) to a sequence of nucleotides of corresponding length in the target oligonucleotide, e.g. the Pro-N-Cadherin mRNA. The complementary region of the nucleotide sequence may have any length, but is preferably at least 5, and optionally no more than 50, nucleotides long, e.g. one of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

Repression of Pro-N-Cadherin expression will preferably result in a decrease in the quantity of Pro-N-Cadherin expressed by a cell, e.g. by a fibroblast or myofibroblast. For example, in a given cell the repression of Pro-N-Cadherin by administration of a suitable nucleic acid will result in a decrease in the quantity of Pro-N-Cadherin expressed by that cell relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function.

A role for the RNAi machinery and small RNAs in targeting of heterochromatin complexes and epigenetic gene silencing at specific chromosomal loci has been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA. RNAi based therapeutics have been progressed into Phase I, II and III clinical trials for a number of indications (Nature 2009 Jan. 22; 457(7228):426-433).

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNAs are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, the present disclosure provides the use of oligonucleotide sequences for down-regulating the expression of Pro-N-Cadherin.

siRNA ligands are typically double stranded and, in order to optimize the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLOS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of Pro-N-Cadherin. Preferably, the shRNA sequence is between 40 and 100 bases in length, more preferably between 40 and 70 bases in length. The stem of the hairpin is preferably between 19 and 30 base pairs in length. The stem may contain G-U pairings to stabilize the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of Pro-N-Cadherin.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific (e.g. heart, liver, kidney or eye specific) promoter. In a further embodiment, the siRNA, longer dsRNA or miRNA is produced exogenously (in vitro) by transcription from a vector.

Suitable vectors may be oligonucleotide vectors configured to express the oligonucleotide agent capable of Pro-N-Cadherin repression. Such vectors may be viral vectors or plasmid vectors. The therapeutic oligonucleotide may be incorporated in the genome of a viral vector and be operably linked to a regulatory sequence, e.g. promoter, which drives its expression. The term “operably linked” may include the situation where a selected nucleotide sequence and regulatory nucleotide sequence are covalently linked in such a way as to place the expression of a nucleotide sequence under the influence or control of the regulatory sequence. Thus a regulatory sequence is operably linked to a selected nucleotide sequence if the regulatory sequence is capable of effecting transcription of a nucleotide sequence which forms part or all of the selected nucleotide sequence.

Viral vectors encoding promoter-expressed siRNA sequences are known in the art and have the benefit of long term expression of the therapeutic oligonucleotide. Examples include lentiviral (Nature 2009 Jan. 22; 457(7228):426-433), adenovirus (Shen et al., FEBS Lett 2003 Mar. 27; 539(1-3)111-4) and retroviruses (Barton and Medzhitov PNAS Nov. 12, 2002 vol. 99, no. 23 14943-14945).

In other embodiments a vector may be configured to assist delivery of the therapeutic oligonucleotide to the site at which repression of Pro-N-Cadherin expression is required. Such vectors typically involve complexing the oligonucleotide with a positively charged vector (e.g., cationic cell penetrating peptides, cationic polymers and dendrimers, and cationic lipids); conjugating the oligonucleotide with small molecules (e.g., cholesterol, bile acids, and lipids), polymers, antibodies, and RNAs; or encapsulating the oligonucleotide in nanoparticulate formulations (Wang et al., AAPS J. 2010 December; 12(4): 492-503).

In one embodiment, a vector may comprise a nucleic acid sequence in both the sense and antisense orientation, such that when expressed as RNA the sense and antisense sections will associate to form a double stranded RNA.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2′-O-alkyl; 2′-O-allyl; 2′-S-alkyl; 2′-S-allyl; 2′-fluoro-; 2′-halo or azido-ribose, carbocyclic sugar analogues, a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine, 1-methylcytosine.

Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498).

Accordingly, the present disclosure provides a nucleic acid that is capable, when suitably introduced into or expressed within a mammalian, e.g. human, cell that otherwise expresses Pro-N-Cadherin, of suppressing Pro-N-Cadherin expression by RNAi.

The nucleic acid may have substantial sequence identity to a portion of Pro-N-Cadherin mRNA, or the complementary sequence to said mRNA.

The nucleic acid may be a double-stranded siRNA. (As the skilled person will appreciate, and as explained further below, a siRNA molecule may include a short 3′ DNA sequence also.)

Alternatively, the nucleic acid may be a DNA (usually double-stranded DNA) which, when transcribed in a mammalian cell, yields an RNA having two complementary portions joined via a spacer, such that the RNA takes the form of a hairpin when the complementary portions hybridize with each other. In a mammalian cell, the hairpin structure may be cleaved from the molecule by the enzyme DICER, to yield two distinct, but hybridized, RNA molecules.

Only single-stranded (i.e. non self-hybridized) regions of an mRNA transcript are expected to be suitable targets for RNAi. It is therefore proposed that other sequences very close in the Pro-N-Cadherin mRNA transcript may also be suitable targets for RNAi.

Accordingly, the present disclosure provides nucleic acids that are capable, when suitably introduced into or expressed within a mammalian cell that otherwise expresses Pro-N-Cadherin, of suppressing Pro-N-Cadherin expression by RNAi, wherein the nucleic acid is generally targeted to the sequence of, or portion thereof, of Pro-N-Cadherin.

By “generally targeted” the nucleic acid may target a sequence that overlaps with Pro-N-Cadherin. In particular, the nucleic acid may target a sequence in the mRNA of human Pro-N-Cadherin that is slightly longer or shorter than one of Pro-N-Cadherin, but is otherwise identical to the native form.

It is expected that perfect identity/complementarity between the nucleic acid of the invention and the target sequence, although preferred, is not essential. Accordingly, the nucleic acid of the invention may include a single mismatch compared to the mRNA of Pro-N-Cadherin. It is expected, however, that the presence of even a single mismatch is likely to lead to reduced efficiency, so the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the invention that include non-natural nucleotides. In one embodiment, the nucleic acid (herein referred to as double-stranded siRNA) includes the double-stranded RNA sequences for Pro-N-Cadherin.

However, it is also expected that slightly shorter or longer sequences directed to the same region of Pro-N-Cadherin mRNA will also be effective. In particular, it is expected that double-stranded sequences between 17 and 23 bp in length will also be effective.

The strands that form the double-stranded RNA may have short 3′ dinucleotide overhangs, which may be DNA or RNA. The use of a 3′ DNA overhang has no effect on siRNA activity compared to a 3′ RNA overhang, but reduces the cost of chemical synthesis of the nucleic acid strands (Elbashir et al., 2001c). For this reason, DNA dinucleotides may be preferred.

When present, the dinucleotide overhangs may be symmetrical to each other, though this is not essential. Indeed, the 3′ overhang of the sense (upper) strand is irrelevant for RNAi activity, as it does not participate in mRNA recognition and degradation (Elbashir et al., 2001a, 2001b, 2001c).

While RNAi experiments in Drosophila show that antisense 3′ overhangs may participate in mRNA recognition and targeting (Elbashir et al. 2001c), 3′ overhangs do not appear to be necessary for RNAi activity of siRNA in mammalian cells. Incorrect annealing of 3′ overhangs is therefore thought to have little effect in mammalian cells (Elbashir et al. 2001c; Czauderna et al. 2003).

Any dinucleotide overhang may therefore be used in the antisense strand of the siRNA. Nevertheless, the dinucleotide is preferably -UU or -UG (or -TT or -TG if the overhang is DNA), more preferably -UU (or -TT). The -UU (or -TT) dinucleotide overhang is most effective and is consistent with (i.e. capable of forming part of) the RNA polymerase III end of transcription signal (the terminator signal is TTTTT). Accordingly, this dinucleotide is most preferred. The dinucleotides AA, CC and GG may also be used, but are less effective and consequently less preferred.

Moreover, the 3′ overhangs may be omitted entirely from the siRNA.

The present disclosure also provides single-stranded nucleic acids (herein referred to as single-stranded siRNAs) respectively consisting of a component strand of one of the aforementioned double-stranded nucleic acids, preferably with the 3′-overhangs, but optionally without. The present disclosure also provides kits containing pairs of such single-stranded nucleic acids, which are capable of hybridizing with each other in vitro to form the aforementioned double-stranded siRNAs, which may then be introduced into cells.

The present disclosure also provides DNA that, when transcribed in a mammalian cell, yields an RNA (herein also referred to as an shRNA) having two complementary portions which are capable of self-hybridizing to produce a double-stranded motif or a sequence that differs from any one of the aforementioned sequences by a single base pair substitution.

The complementary portions will generally be joined by a spacer, which has suitable length and sequence to allow the two complementary portions to hybridize with each other. The two complementary (i.e. sense and antisense) portions may be joined 5′-3′ in either order. The spacer will typically be a short sequence, of approximately 4-12 nucleotides, preferably 4-9 nucleotides, more preferably 6-9 nucleotides.

Preferably the 5′ end of the spacer (immediately 3′ of the upstream complementary portion) consists of the nucleotides -UU- or -UG-, again preferably -UU- (though, again, the use of these particular dinucleotides is not essential). A suitable spacer, recommended for use in the pSuper system of OligoEngine (Seattle, Wash., USA) is UUCAAGAGA. In this and other cases, the ends of the spacer may hybridize with each other.

Similarly, the transcribed RNA preferably includes a 3′ overhang from the downstream complementary portion. Again, this is preferably —UU or -UG, more preferably -UU.

Such shRNA molecules may then be cleaved in the mammalian cell by the enzyme DICER to yield a double-stranded siRNA as described above, in which one or each strand of the hybridized dsRNA includes a 3′ overhang.

Techniques for the synthesis of the nucleic acids of the invention are of course well known in the art.

The skilled person is well able to construct suitable transcription vectors for the DNA of the present disclosure using well-known techniques and commercially available materials. In particular, the DNA will be associated with control sequences, including a promoter and a transcription termination sequence.

Of particular suitability are the commercially available pSuper and pSuperior systems of OligoEngine (Seattle, Wash., USA). These use a polymerase-III promoter (H1) and a T5 transcription terminator sequence that contributes two U residues at the 3′ end of the transcript (which, after DICER processing, provide a 3′ UU overhang of one strand of the siRNA).

Another suitable system is described in Shin et al. (RNA, 2009 May; 15(5): 898-910), which uses another polymerase-III promoter (U6).

The double-stranded siRNAs of the present disclosure may be introduced into mammalian cells in vitro or in vivo using known techniques, as described below, to suppress expression of Pro-N-Cadherin.

Similarly, transcription vectors containing the DNAs of the present disclosure may be introduced into tumor cells in vitro or in vivo using known techniques, as described below, for transient or stable expression of RNA, again to suppress expression of Pro-N-Cadherin.

Accordingly, the present disclosure also provides a method of suppressing Pro-N-Cadherin expression in a mammalian, e.g. human, cell, the method comprising administering to the cell a double-stranded siRNA of the present disclosure or a transcription vector of the present disclosure.

Similarly, the present disclosure further provides a method of treating fibrosis, the method comprising administering to a subject a double-stranded siRNA of the invention or a transcription vector of the present disclosure.

The present disclosure further provides the double-stranded siRNAs of the present disclosure and the transcription vectors of the invention, for use in a method of treatment, preferably a method of treating fibrosis.

The present disclosure further provides the use of the double-stranded siRNAs of the present disclosure and the transcription vectors of the invention in the preparation of a medicament for the treatment of fibrosis.

The present disclosure further provides a composition comprising a double-stranded siRNA of the present disclosure or a transcription vector of the invention in admixture with one or more pharmaceutically acceptable carriers. Suitable carriers include lipophilic carriers or vesicles, which may assist in penetration of the cell membrane.

Materials and methods suitable for the administration of siRNA duplexes and DNA vectors of the present disclosure are well known in the art and improved methods are under development, given the potential of RNAi technology.

Generally, many techniques are available for introducing nucleic acids into mammalian cells. The choice of technique will depend on whether the nucleic acid is transferred into cultured cells in vitro or in vivo in the cells of a patient. Techniques suitable for the transfer of nucleic acid into mammalian cells in vitro include the use of liposomes, electroporation, microinjection, cell fusion, DEAE dextran and calcium phosphate precipitation. In vivo gene transfer techniques include transfection with viral (typically retroviral) vectors and viral coat protein-liposome mediated transfection (Dzau et al. (2003) Trends in Biotechnology 11, 205-210).

In therapeutic applications, agents capable of inhibiting the action of Pro-N-Cadherin or agents capable of preventing or reducing the expression of Pro-N-Cadherin are preferably formulated as a medicament or pharmaceutical together with one or more other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilizers, solubilizers, surfactants (e.g., wetting agents), masking agents, coloring agents, flavoring agents, and sweetening agents.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, adjuvant, excipient, etc. must also be “acceptable” in the sense of being compatible with the other ingredients of the formulation.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulations may be prepared for topical, parenteral, systemic, intravenous, intra-arterial, intramuscular, intrathecal, intraocular, intra-conjunctival, subcutaneous, oral or transdermal routes of administration which may include injection. Injectable formulations may comprise the selected agent in a sterile or isotonic medium.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

In some embodiments, kits for carrying out the methods described herein are provided. The kits provided may contain the necessary components with which to carry out one or more of the above-noted methods. In one embodiment, a kit for detecting a biomarker specific for pathological fibroblast cells are provided. The kit may comprise an antibody specific to the biomarker. In a preferred embodiment, the biomarker is pro-N-cadherin. In some embodiments, the detecting is by an antibody specific to the biomarker. In other embodiments, the detecting is by other methods described herein. In one embodiment, the kit comprises an antibody to pro-N-cadherin conjugated to a detection agent or magnetic beads. In further embodiments, a control is provided. In one embodiment, the control is a positive control, for example, a sample positive for the biomarker specific for the fibroblast cells expressing pro-N-cadherin. In another example, the control is a control obtained from a healthy individual that does not have such pathological fibroblast cells.

In further embodiments, the kits may include a composition for the treatment of a subject in which pathological fibroblast cells have been detected. The kits may include an antibody specific to the biomarker (e.g. pro-N-cadherin antibody). In some further embodiments, the kit may further include one or more additional therapeutic agents (e.g., therapeutic agents for treating fibrosis or conditions associated with pathological fibroblast cells).

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

### Materials and Methods

### Cell Culture and Reagents

All cells were cultured at 37° C., 5% CO₂ in a humidified chamber. Cells were used between passages 3 and 10 and validated for alpha-smooth muscle actin (α-SMA) expression prior to use in experiments by immunoblot using α-SMA antibody (Cell Signaling 19245S, Danvers, MA, USA). The LL97A (ATCC CCL-191, Manassas, VA, USA), LL29 (ATCC CCL-134, Manassas, VA, USA) and CCD-16Lu (ATCC CCL-204, Manassas, VA, USA) cell lines were purchased from ATCC and grown by their specifications. Primary ventricular normal human cardiac fibroblasts (NHCF) from a healthy donor heart were purchased from Lonza (CC-2904, Morrisville, NC, USA) and grown in their recommended media. Primary ventricular cardiac fibroblasts (DCM-CF) from explant failed human hearts were isolated from tissue obtained from the Duke Human Heart Repository (IRB #Pro87831) and grown in DMEM; 10% FBS; 1× Pen Strep. Primary normal human lung fibroblasts (NHLF) were purchased from the Duke Cell Culture Facility (Lonza CC-2512) and grown in MEM; 10% FBS; 1× Pen Strep.

pro-N-Cadherin Antibody Purification

pro-N-cadherin monoclonal antibody-producing hybridoma clone 10A10 was a gift from the Wahl Laboratory at the University of Nebraska. Cells were acclimated to Hybridoma-SFM (Gibco, Grand Island, NE, USA) supplemented with 10% super low IgG HI-FBS (Hyclone, Logan, UT, USA) and grown to confluency. Hybridoma cells were passaged, and the media was replaced with serum free Hybridoma-SFM media and allowed to express for 5 days. Supernatant was collected by centrifugation, passed through a 0.22 μm filter and antibodies were purified by affinity chromatography using protein G sepharose prepacked column (GE Healthcare, Salt Lake City, UT, USA) following the manufacturer's protocol using the Amersham Biosciences AKTA FPLC system. Antibodies were dialyzed into final buffer PBS pH 7.4 and stored at −20° C. for these studies.

Fibroblast Isolation from Explant Tissues

Tissues were minced into small pieces and enzymatically digested in appropriate volume of tissue dissociation solution comprised of PBS pH 7.4, 5 mg/mL Collagenase Type IV (Gibco, Grand Island, NE, USA), 1.3 mg/mL Dispase II (Gibco, Grand Island, NE, USA), 0.05% Trypsin, with agitation at 37° Celsius for 1 h. After incubation in dissociation solution, 25 mL of HBSS (Gibco, Grand Island, NE, USA) was added, followed by serial pipetting for manual dissociation and centrifugation at 1200 RPM, 4° C., for 10 min. The supernatant was then aspirated and the pellet was resuspended in 5 mL HBSS then passed through a 100 μm filter followed by a 70 μm filter. The filtered cell suspension was then centrifuged at 1200 RPM, 4° C., for 5 min, the supernatant was aspirated and the pellet resuspended into DMEM; 10% FBS; 1× Pen Strep. Cells were plated and passaged to remove plasma and myocyte contaminant.

### Tissue Procurement and Processing

Fresh explant cardiac tissues were received from the Duke HHR (IRB Pro00087831) within 12 h of explant in PBS pH 7.4, 1× Pen Strep (Gibco, Grand Island, NE, USA), on ice and processed same day. Tissues were divided and a portion was immediately processed for fibroblast isolation (above). The remaining portion was fixed overnight at room temperature in 4% paraformaldehyde and PBS pH 7.4 followed by 70% EtOH and embedded in paraffin. Additional flash-frozen cardiac tissues from the Duke HHR, including a subset of previously characterized samples [Schechter, M. A.; Hsieh, M. K. H.; Njoroge, L. W.; Thompson, J. W.; Soderblom, E. J.; Feger, B. J.; Troupes, C. D.; Hershberger, K.; Ilkayeva, O. R.; Nagel, W. L.; et al. Phosphoproteomic Profiling of Human Myocardial Tissues Distinguishes Ischemic from Non-Ischemic End Stage Heart Failure. PLOS ONE 2014, 9, e104157] were embedded in OCT medium and sectioned on a Leica cryostat into 5 μm sections and adhered to charged glass slides for immunohistochemical analysis. Liver tissue specimens were provided by the DUHS Nonalcoholic Fatty Liver Disease Research Database and Specimen Repository under Pro00005368 and Abdominal Transplant Repository under Pro00107246. Formalin-fixed paraffin embedded heart and liver tissues were cut on a Leica microtome into 3-5 μm sections and adhered to charged glass slides for immunohistochemical analysis. Lung tissue sections were obtained from the Duke Bio Repository and Precision Pathology Center (BRPC) on charged glass slides under an IRB exemption. Additional heart, lung, and liver tissue microarrays were obtained commercially from USA Biomax, Inc. (Rockville, MD, USA) as formalin-fixed paraffin embedded 1.5 mm cores on charged glass slides.

### Immunohistochemistry

Immunohistochemical detection of PNC was performed on formalin fixed paraffin embedded tissue samples sectioned at 3-5 μm. Sections were deparaffinated with xylene, rehydrated, and treated with 3% H₂O₂ to quench endogenous peroxidase. Heat-mediated antigen retrieval was performed in a citrate buffer (pH 6) and blocked with 5% horse serum. PNC was detected using purified m-α-PNC mAb clone 10A10 at 5-10 μg/mL at 4° C. overnight. An avidin-biotin amplification step and chromogenic detection (DAB) of α-mouse HRP-conjugated secondary antibody was used to visualize pro-N-cadherin localization and expression. Tissues were counter-stained with Mayer's hematoxylin and mounted with Cytoseal 60 (Thermo Fisher, Grand Island, NE, USA) mounting media for imaging.

### Plasma Procurement

Healthy human donor plasma was purchased commercially from Innovative Research, Inc, Novi, MI, USA. Plasma from heart failure patients was provided by the Duke HHR IRB Pro00087831. Plasma from NAFLD-cirrhosis patients was provided by the Duke Nonalcoholic Fatty Liver Disease Research Database and Specimen Repository under Pro00005368 and Abdominal Transplant Repository under Pro00107246. Plasma from IPF patients was purchased commercially from Innovative Research, Inc.

### Immunoprecipitations

LL29 conditioned media was incubated at 4° C. with 10 μg of murine 10A10 antibody for 1 h. Protein G Sepharose (GE Healthcare, Salt Lake City, UT, USA) was blocked with 1% BSA in PBS then 5 μL of resin was added to the conditioned media. After incubating at 4° C. for 30 min, the resin was washed three times with PBS then eluted using 1×LDS sample buffer (Thermo Fisher, Grand Island, NE, USA) with B-mercaptoethanol. Elutes were boiled, immunoblotted, and developed using humanized 10A10 chimeric antibody.

For plasma samples, 10A10 and mouse IgG1 isotype control (Thermo Fisher, Grand Island, NE, USA) antibodies were first crosslinked to protein G sepharose (GE Healthcare, Salt Lake City, UT, USA) using Pierce crosslink IP kit (Thermo Fisher, Grand Island, NE, USA). Equivalent volumes of healthy donor and diseased donor plasma samples were pooled then diluted 1:10 in PBS, centrifuged at 16,000 G and filtered using a 0.22 μm filter. Samples were then rotated overnight with antibody crosslinked protein G sepharose, washed and eluted following the manufacturer's protocol. Elutes were immunoblotted and developed using murine 10A10 antibody.

### SDS-PAGE and Immunoblotting

Total cell lysates were prepared using RIPA buffer adjusted to 1% w/v SDS (Sigma, Saint Louis, USA) supplemented with Halt Protease & Phosphatase Inhibitor Cocktail (Thermo Fisher, Grand Island, NE, USA) and Benzonase (Millipore Sigma, Saint Louis, MO, USA). Cell surface proteins were isolated using the Pierce cell surface biotinylation and isolation kit (Thermo Fisher, Grand Island, NE, USA) following the manufacturer's protocol. Plasma membrane loading control Na,k-ATPase α-1 (cell signaling 23565T, Massachusetts, USA) was used for cell surface protein isolates. Protein concentration of each lysate was measured using Pierce BCA protein assay (Thermo Fisher, Grand Island, NE, USA). For total lysates, precleared lysates were boiled in sample buffer (Thermo Fisher, Grand Island, NE, USA) and 40 μg of protein was loaded. All samples were run on 10% NuPage gels containing 0.1% SDS under reducing conditions. A discontinuous Laemmli buffer system was used. The proteins were transferred from the gels to nitrocellulose membranes. The molecular weights were assessed using Precision Plus Prestained Marker (Bio-Rad, Hercules, CA, USA). The membranes were thoroughly washed with tris-buffered saline (TBS) and then blocked with infrared blocking buffer (Rockland, Pottstown, PA, USA) for 1 h at room temperature. Membranes were incubated with antibodies overnight at 4° C. in 5% BSA, 1×TBS, 0.1% Tween-20. RPL13A (Cell Signaling 2765S, Danvers, MA USA) was used for total cell lysate loading control, along with mature N-cadherin (Sigma, GC-4 clone, C3865, Saint Louis, MO, USA) and α-PNC mAb. After incubation with the primary antibody, the membranes were washed three times for 5 min each with 1×TBS containing 0.1% Tween 20 (TBST). The membranes were then incubated with the manufacturer's recommended dilution of appropriate Alexa Fluor conjugated secondary (Thermo Fisher, Grand Island, NE, USA). The membranes were washed twice for 5 min each with TBST and once with TBS for 5 min. The probed membranes were scanned on a Li-Cor Odyssey System (Li-Cor Biosciences, Lincoln, NE, USA).

### Flow Cytometry

Cells were plated in complete media in 10 cm dishes and allowed to anchor overnight. The following day, cells were washed with PBS pH 7.4, then detached using PBS pH 7.4; 0.5 mM EDTA; 10% Glycerol at 37° C. for approximately 5-10 min or until cell rounding followed by scraping. Cells were kept on ice for the duration of the staining procedure. Cells were pelleted at 1200 RPM, 4° C., for 5 min followed by supernatant aspiration, PBS pH 7.4 wash, and resuspension in PBS pH 7.4; 1% BSA; 0.09% sodium azide. Cells were incubated with either α-N-cadherin antibody (Sigma, GC-4 clone, C3865), α-PNC antibody (5 μg/mL), or 5 μg/mL mouse IgG1 isotype control (Thermo Fisher, Grand Island, NE, USA) for 30 min. Cells were washed with PBS pH 7.4; 1% BSA; 0.09% sodium azide followed by 5 μg/mL Alexa Fluor 488 secondary antibody (Thermo Fisher, Grand Island, NE, USA) incubation for 30 min. Cells were then washed with PBS pH 7.4; 1% BSA; 0.9% sodium azide and stained with 7AAD (BioLegend, San Diego, CA, USA) following the manufacturer's protocol. Cells were analyzed using the Guava EasyCyte (Luminex, Austin, TX, USA) flow cytometer and the latest version of Flowjo software, gating and excluding 7AAD positive cells. At least 20,000 events were collected for each experiment. To calculate background, the same sample was ran using the isotype control antibody four times independently and histograms were stacked to determine baseline chi-square and SE dymax % positive.

### Immunofluorescent Microscopy

For immunofluorescent microscopy, cells were plated into multi-well chamber slides coated with 5 μg/mL human Collagen type I (Sigma, Saint Louis, MO, USA) in PBS. After cells were allowed to adhere overnight, each well was aspirated, washed with PBS pH 7.4 and fixed in 1% formaldehyde for 30 min at room temperature. For perinuclear PNC staining, cells were permeabilized using 0.05% Triton in PBS. Cells were then blocked with 5% goat serum (Abcam, Cambridge, UK); PBS pH 7.4 for 1 h at room temperature. Primary antibody against pro-N-cadherin was incubated at 2 μg/mL on cells overnight at 4° C. followed by Qdot 655 conjugated secondary (Thermo Fisher, Grand Island, NE, USA) for 1 h at room temperature. For colocalization, FN1 antibody (Cell Signaling 26836S, Danvers, MA, USA) was used following the manufacturer's protocol followed by AlexaFluor 488 conjugated secondary (Thermo Fisher, Grand Island, NE, USA). DAPI was used following the manufacturers protocol (Sigma, Saint Louis, MO, USA) to visualize DNA. Coverslips were applied using 50% glycerol in PBS and sealed with nail polish. Images were taken using the Leica DMI400 B.

### Sandwich Enzyme-Linked Immunosorbent Assay

Recombinant prodomain (rPro) of N-cadherin, amino acids 26-159 (Accession #AAB22854) was generated and supplied by GenScript (Piscataway, NJ, USA) and used to optimize a PNC sandwich enzyme-linked immunosorbent assay (ELISA) and later used as an antagonist to cell surface PNC in migration assays. High binding ELISA plates (Costar, Kennebunk, ME, USA) were used to bind 1 μg/well of α-PNC antibody 10A10 as the capture antibody. Washes were performed using PBS pH 7.4 0.1% Tween 20. Three washes (300 μL/well) were performed between each of the following steps using Biotek (Winooski, VT, USA) ELx405 Select CW automated plate washer. All steps were performed at room temperature with room temperature equilibrated buffers. Capture antibody was bound overnight at room temperature in PBS pH 7.4 followed by blocking with 300 μL per well blocking buffer 5% non-fat dry milk (Bio-Rad, Hercules, CA, USA) in 1×PBS (Gibco, Grand Island, NE, USA) with 0.1% Tween 20 for 1 h. Plasma samples and rPro analyte standard were applied 100 μL per well in 1% BSA, PBS pH 7.4, 5 mM EDTA, 0.1% Tween 20 for 1 h followed by 100 μL/well 1:800 dilution of biotinylated polyclonal sheep α-PNC detection antibody (R&D BAF1388, Minneapolis, MN, USA) in 1% BSA, PBS pH 7.4, 0.1% Tween 20 for 1 h. Streptavidin horseradish peroxidase conjugate (Thermo Fisher, Grand Island, NE, USA) was applied at 100 μL per well, 1:800 in 2% BSA, PBS pH 7.4 for 20 min followed by 150 μL of ABTS (Thermo Fisher, Grand Island, NE, USA) for 15 min and read using the Biotek (Winooski, VT, USA) Cytation 3 Imager Reader at absorbance (Abs) 410 nm.

### Solid Phase Enzyme Immunoassays

To find rPro binding partners, a solid phase enzyme immunoassay was used. Medium binding ELISA plates (Costar, Kennebunk, ME, USA) were coated with either 10 μg/mL human plasma fibronectin (Sigma, Saint Louis, MO, USA), human type I collagen (Sigma, Saint Louis, MO, USA) or human type III collagen (Sigma, Saint Louis, MO, USA) overnight at room temperature in PBS. All washes were done with PBS 0.1% Tween-20, 300 μL/well, three times using the Biotek ELx405 Select CW automated plate washer. After immobilizing fibronectin, collagen type I, or BSA, wells were washed then blocked using 300 μL/well 1% BSA PBS in wash buffer for 1 h at room temperature. Wells were washed then a serial dilution starting at 10 μg/mL of His tagged rPro peptide diluted in blocking buffer was incubated over the immobilized proteins for one hour at room temperature. Wells were washed and incubated with 1:2000 mouse α-His biotinylated antibody (Invitrogen MA1-21315-BTIN, Grand Island, NE, USA) in block for 1 h at room temperature followed by 1:2000 streptavidin HRP (Thermo Fisher, Grand Island, NE, USA) in 2% BSA PBS at room temperature for 15 min. Immunoassay was developed using TMB Ultra (R&D, Minneapolis, MN, USA) following the manufacturers recommended protocol. Absorbance 450 nm was read using the Biotek Cytation 3 indicating bound his tagged rPro peptide.

For antibody displacement assays, medium binding ELISA plates (Costar, Kennebunk, ME, USA) were coated with human plasma fibronectin (Sigma, Saint Louis, MO, USA) at 10 μg/mL in PBS overnight at room temperature. All washes were done with PBS 0.1% Tween-20, 300 μL/well, three times using the Biotek ELx405 Select CW automated plate washer. After immobilizing fibronectin, wells were washed then blocked using 300 μL/well 5% human plasma in wash buffer for 1 h at room temperature. While blocking, antibodies were added at the respective concentration to blocking buffer containing 1.5 μg/mL recombinant his-tagged prodomain. Wells were washed and samples were applied for 1 h at room temperature. Wells were washed and incubated with 1:2000 mouse α-His biotinylated antibody (Invitrogen MA1-21315-BTIN, Grand Island, NE, USA) for 1 h at room temperature followed by 1:2000 streptavidin HRP (Thermo Fisher, Grand Island, NE, USA) at room temperature for 15 min. Immunoassay was developed using TMB Ultra (R&D, Minneapolis, MN, USA) following the manufacturers recommended protocol. Absorbance 450 nm was read using the Biotek Cytation 3 indicating bound his tagged rPro peptide.

Humanization of Murine pro-N-Cadherin Antibody 10A10

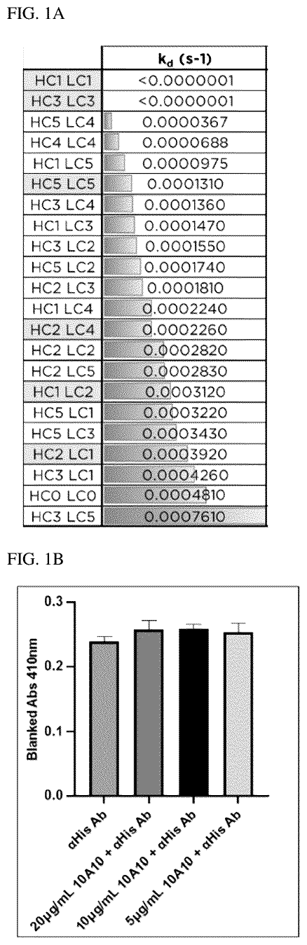

Fusion Antibodies, PLC (Belfast, Northern Ireland) was contracted to humanize the murine α-PNC mAb 10A10. CDR regions of the murine α-PNC mAb were cloned onto human IgG4 heavy and light chain constant domains. The variable regions of the murine α-PNC mAb were modified in-silico to reduce T-cell epitope antigenicity and increase binding affinity to the prodomain of N-cadherin. These 22 humanized antibody designs were expressed, purified, and ranked for binding affinity using bio-layer interferometry (octet) technology by Fusion Antibodies to the rPro peptide (FIG. 1).

### 2.14. Migration Assays

Transwell permeable supports with a 6.5 mm polycarbonate membrane and 8 μm pores were used to separate the upper and lower chambers of a 24-well plate. Both sides of the membrane were coated with 1 μg/mL human plasma fibronectin (Sigma, Saint Louis, MO, USA). Bovine fibronectin was depleted from the complete media using gelatin Sepharose (GE Healthcare, Salt Lake City, UT, USA) following the manufacturers protocol. Complete media containing 10 ng/ml TGF-β1 was then added to the lower chamber at 600 μL/well. Cells were trypsinized, pelleted, resuspended in low serum media containing 0.5% FBS and added into the upper chamber at 1.0×10⁴ cells/well in 100 μL media per well. After allowing attachment, antibody treatment, rPro or PBS blank was added to the lower chamber and the plate was incubated for 5 h at 37° ° C., 5% CO₂. Media was aspirated, and cells were fixed using 4% Formaldehyde for 15 min at room temperature. Cells were removed from the top of the upper chamber using a sterile cotton swab and wells were washed three times with PBS. Adherent cells on the apical side of wells were then stained by applying DAPI solution (Sigma, Saint Louis, MO, USA) following the manufacturers protocol. Nuclei were visualized by fluorescent microscopy and counted using the Biotek Cytation 3 and latest Gen5 v 3.11 software (Winooski, VT, USA). Samples were imaged using the 2.5× objective and total cell numbers were counted in each frame.

### Statistical Analysis

Statistical analysis was performed using T-tests and one-way ANOVA with Post hoc multiple comparisons analyses where appropriate using the latest version of GraphPad Prism 9 (San Diego, CA, USA) software (*p≤0.05, **p≤0.01, ***p≤0.001, ****p≤0.0001). Post hoc analyses are indicated in corresponding figure legends. For flow cytometry, chi-squared test was performed using Flowjo v 10.7.2 (Vancouver, BC, Canada) analysis software. Chi-squared ≥4 is statistically significant.

### Results

pro-N-Cadherin Is Aberrantly Localized in Failing Tissue from Heart, Lung, and Liver and Expressed on the Surface of Isolated Myofibroblasts from Pathological Origins

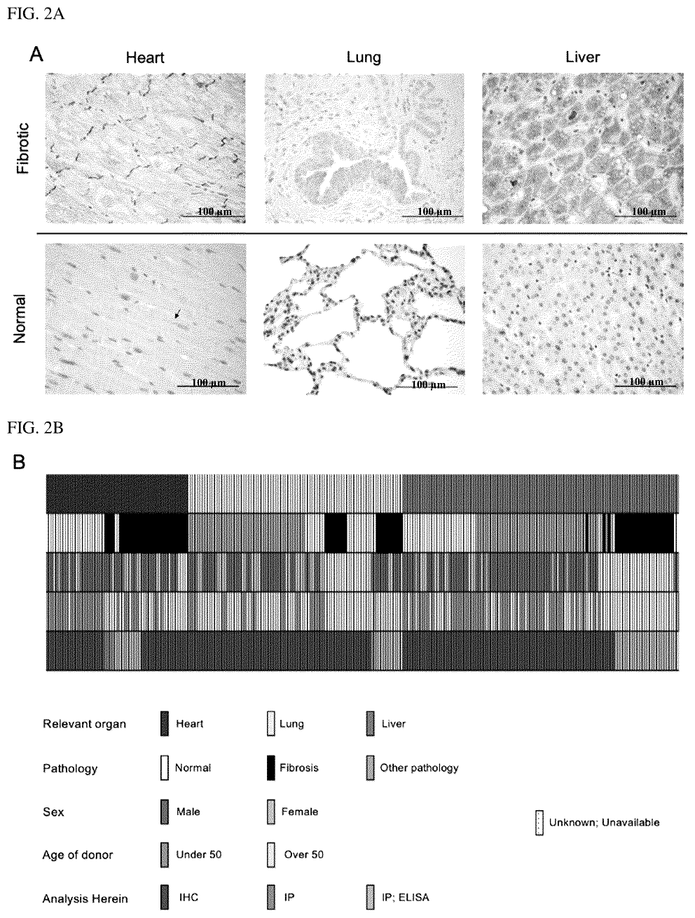

The role of the classical cadherin CDH2 or N-cadherin is well established in normal cardiac function as well as in the pathogenesis of a number of cardiac diseases and disorders (Kostetskii, I.; Li, J.; Xiong, Y.; Zhou, R.; Ferrari, V. A.; Patel, V. V.; Molkentin, J. D.; Radice, G. L. Induced Deletion of the N-cadheringene in the Heart Leads to Dissolution of the Intercalated Disc Structure. Circ. Res. 2005, 96, 346-354; Chopra, A.; Tabdanov, E.; Patel, H.; Janmey, P. A.; Kresh, J. Y. Cardiac myocyte remodeling mediated by N-cadherin-dependent mechanosensing. Am. J. Physiol. Circ. Physiol. 2011, 300, H1252-H1266; Vite, A.; Radice, G. L. N-cadherin/catenin Complex as a Master Regulator of Intercalated Disc Function. Cell Commun. Adhes. 2014, 21, 169-179). Given that N-cadherin signaling is critical for cardiac function and involved in cardiac remodeling, we investigated whether the precursor, proprotein pro-N-cadherin (PNC) plays a role in the pathogenesis of cardiac disease [Chopra, A.; Tabdanov, E.; Patel, H.; Janmey, P. A.; Kresh, J. Y. Cardiac myocyte remodeling mediated by N-cadherin-dependent mechanosensing. Am. J. Physiol. Circ. Physiol. 2011, 300, H1252-H1266]. A group of 15 cardiac tissue samples representing dilated non-ischemic cardiomyopathy, hypertrophic cardiomyopathy, and ischemic infarcted cardiomyopathy were obtained from the Duke University Human Heart Repository (HHR). Samples were deidentified, blinded, and stained for the presence of PNC using a previously characterized murine anti-PNC monoclonal antibody (m-α-PNC mAb) clone 10A10 that binds specifically to the precursor, prodomain of N-cadherin [Wahl, J. K.; Kim, Y. J.; Cullen, J. M.; Johnson, K. R.; Wheelock, M. J. N-cadherin-Catenin Complexes Form Prior to Cleavage of the Proregion and Transport to the Plasma Membrane. J. Biol. Chem. 2003, 278, 17269-17276]. Each failed, fibrotic tissue shows strong expression of PNC, notably at the intercalated discs where mature N-cadherin would be expected in normal physiology in the heart [Tsuchiya, B.; Sato, Y.; Kameya, T.; Okayasu, I.; Mukai, K. Differential expression of N-cadherin and E-cadherin in normal human tissues. Arch. Histol. Cytol. 2006, 69, 135-145], in the bronchiolar epithelium of fibrotic lungs, and in hepatocytes of fibrotic livers (FIG. 2).

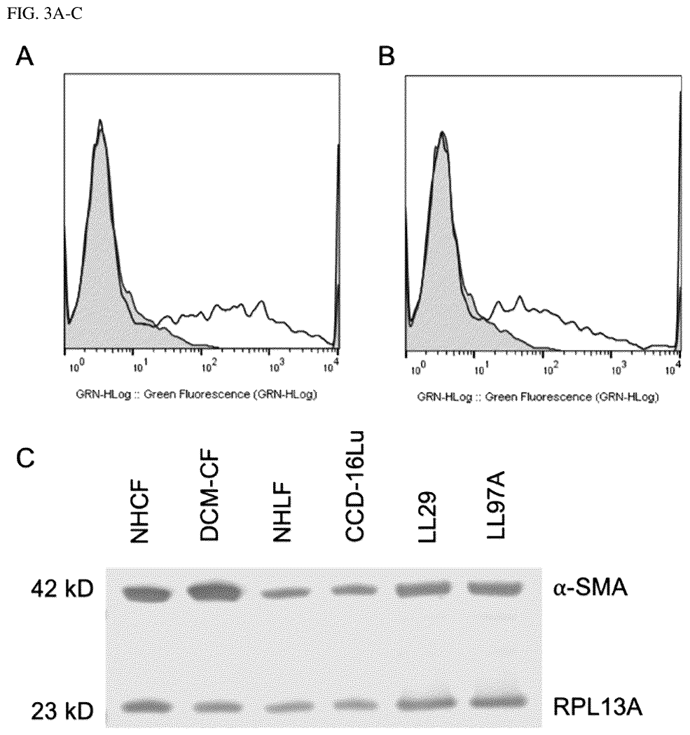

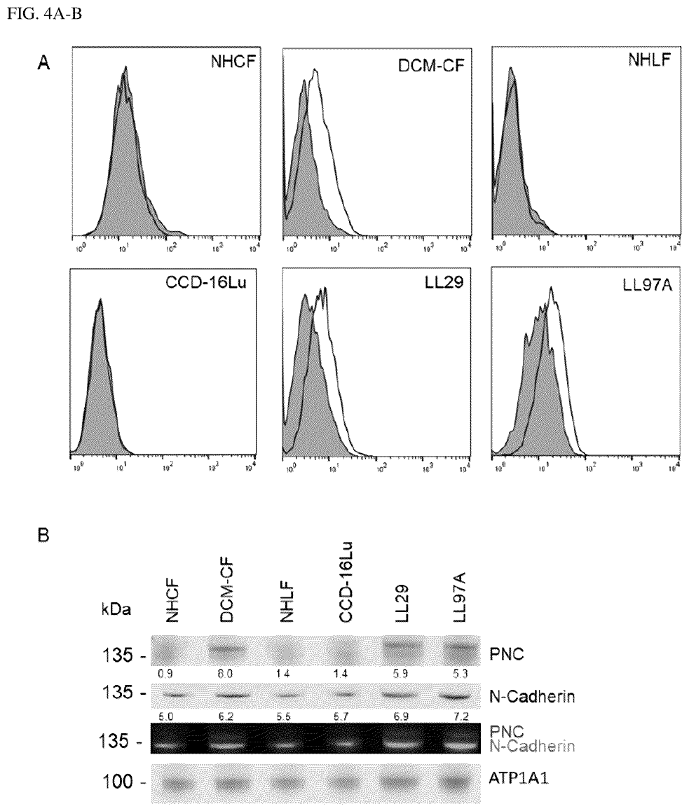

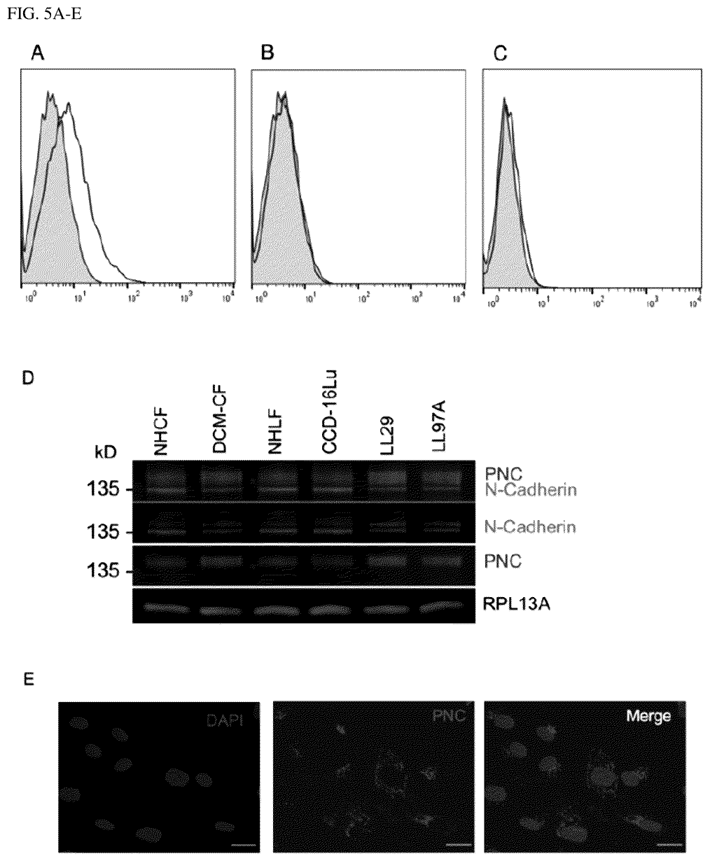

To validate the specific presence of aberrantly localized PNC in the case of failing cardiac tissue, a sample of healthy human heart tissue, obtained by surgical excision during a heart transplant procedure, was processed and stained for the presence of PNC, in addition to a human tissue microarray of 24 unique samples of healthy human heart tissue. While cardiac tissue is known to have high protein expression of mature N-cadherin, we observed no aberrantly localized PNC in the healthy hearts. Perinuclear staining found in healthy cardiac tissue is consistent with normal physiology and N-cadherin processing. These data indicate that aberrant PNC localization in the myocardium is a potential biomarker of the failing heart. Tissues that were obtained fresh immediately post-explant were divided, mechanically and enzymatically digested, and isolated as cell monolayers. These isolated fibroblasts were positive for myofibroblast markers α-SMA and type I collagen by flow cytometry (FIG. 3A, 3B). Additionally, all other fibroblast cell lines used expressed α-SMA protein as is expected with the emergence of the myofibroblast phenotype in vitro [Baum, J.; Duffy, H. S. Fibroblasts and Myofibroblasts: What Are We Talking About? J. Cardiovasc. Pharmacol. 2011, 57, 376-379] (FIG. 3C). Consistent with the aberrant expression observed in the cardiac tissues, we show PNC on the cell surface of myofibroblasts isolated from explant fibrotic cardiac tissue (FIG. 4A, DCM-CF), but not on cardiac myofibroblasts isolated from the healthy donor (FIG. 4A, NHCF).

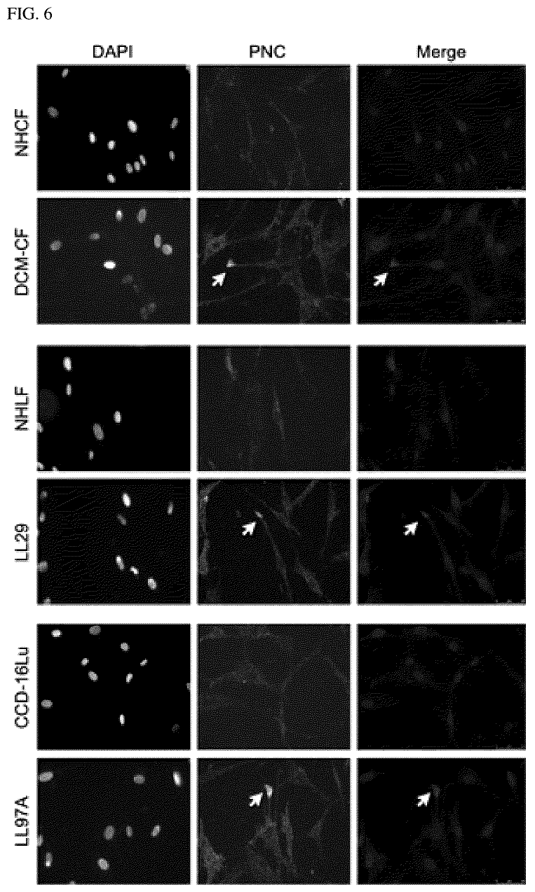

To test the generalization of PNC as a potential biomarker for fibrosis, we expanded our investigation to include two additional organs that are commonly associated with fibrosis-related morbidity. A group of lung and liver tissues were examined representing idiopathic pulmonary fibrosis (IPF) and non-alcoholic fatty liver disease with cirrhosis (NAFLD-cirrhosis). Consistent with the phenotype observed in cardiac explant tissue, PNC is aberrantly localized in all fibrotic tissues. All normal tissues examined show no aberrant expression of PNC, indicating that the aberrant localization of PNC is a potential marker of fibrotic pathology (FIG. 2). To further validate the cell surface phenotype observed in cardiac myofibroblasts, we obtained two additional commercially available cell lines of IPF etiology, LL97A and LL29, and again show that PNC is present on the surface of myofibroblasts isolated from fibrotic tissues (FIG. 4A, DCM-CF, LL97A, LL29) and not on myofibroblasts isolated from healthy tissues (FIG. 4A, NHCF, NHLF, CCD-16Lu). Mature N-cadherin was also analyzed in a representative cell line (FIG. 5A) to confirm no cross reactivity between the PNC antibodies used in these studies (FIG. 5B,C and mature N-cadherin protein. Total lysates of each cell line were immunoblotted for N-cadherin and PNC to corroborate specificity of the PNC antibodies. No cross reactivity between α-PNC antibody and mature N-cadherin is observed (FIG. 5D). Cell surface proteins were isolated from each cell line and immunoblotted for mature N-cadherin and PNC protein. All cell lines express cell surface N-cadherin as expected; however, PNC is only detected in cell surface protein isolates from myofibroblasts derived from fibrotic tissues (FIG. 4B).

Consistent with the flow cytometry results, pathological myofibroblasts immunostained for PNC contain a population of cells with a cell surface PNC positive phenotype. PNC is observed localized to cellular protrusions, indicative of a migratory cell phenotype (FIG. 6). The presence and localization of cell surface PNC on cellular protrusions of myofibroblasts isolated from fibrotic tissues suggests PNC is a possible marker of myofibroblasts' invasive pathology.

pro-N-Cadherin Is Released into Circulation and Quantifiable by Newly Developed ELISA

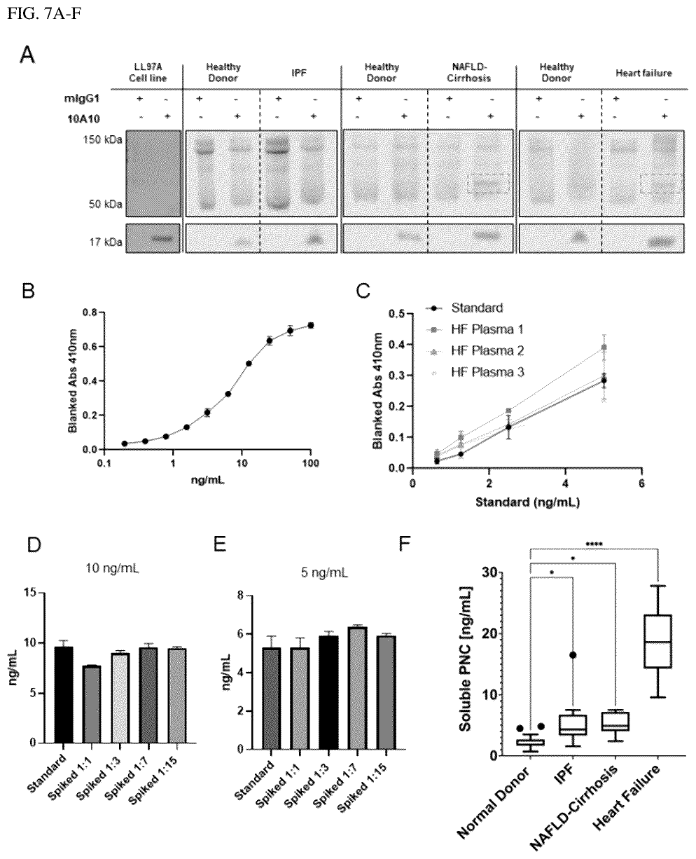

Mature N-cadherin is solubilized into the plasma under normal physiologic conditions [v Derycke, L.; De Wever, O.; Stove, V.; Vanhoecke, B.; Delanghe, J.; Depypere, H.; Bracke, M. Soluble N-cadherin in human biological fluids. Int. J. Cancer 2006, 119, 2895-2900]. We hypothesize aberrantly expressed surface PNC is also detectable in the circulation under pathophysiological conditions. We first analyzed the supernatant of cells cultured in vitro for the presence of soluble PNC (sPNC) and feasibility of detection in patient plasma. To verify its presence, sPNC was immunoprecipitated from conditioned media of LL29 myofibroblasts using m-α-PNC mAb 10A10. A 17 kDa product is shown, consistent with the molecular weight of the prodomain of N-cadherin (FIG. 7A). We next procured plasma from healthy donors and patients with fibrotic cardiomyopathy, IPF and NAFLD-cirrhosis. The immunoprecipitation of PNC from these samples reveals the 17 kDa prodomain in all plasmas tested and a 60 kDa protein in the plasmas from patients with cardiac fibrosis and NAFLD-cirrhosis (FIG. 7A). With these data, we show that PNC is detectable and quantifiable in solution.

After observing solubilized PNC, we sought to optimize and validate a PNC binding sandwich enzyme-linked immunosorbent assay (ELISA). Murine α-PNC monoclonal antibody 10A10 was bound to plates and used as capture antibody and a commercially available biotinylated sheep polyclonal α-PNC antibody was used for the detection antibody. Optimization of capture and detection antibody concentrations was performed using maximal and minimal concentrations of recombinant PNC analyte. A serial dilution of analyte, beginning at 100 ng/mL, was used to determine the range of the standard (FIG. 7B). Plasma samples from patients with cardiomyopathy were obtained from the Duke HHR. Dilution linearity of endogenous analyte from three patient plasma samples were analyzed after serial dilution of plasma in standard diluent (FIG. 7C). Endogenous analyte from failing heart patient plasma diluted linearly (r²≥0.99). Accuracy of the assay was assessed by recovery of recombinant prodomain-spiked healthy donor plasma relative to the standard back-calculations. Healthy donor plasma diluted 1:1, 1:3, 1:7 and 1:15 in standard diluent was spiked with 10 ng/ml (FIG. 7D) and 5 ng/ml (FIG. 7E) recombinant prodomain and assayed. All dilutions were quantified and within the accepted range of 20 percent±the standard back calculations at concentrations 10 ng/ml and 5 ng/mL, validating a novel ELISA for sPNC detection.

Serological Assessment Suggests Cell Surface Release of pro-N-Cadherin from Fibrotic Tissues

To assess sPNC as a potential serological biomarker of fibrosis, we obtained and compared sPNC concentrations in healthy donor plasma to plasma drawn from patients with fibrotic cardiomyopathy. The cohort of samples consists of patients diagnosed with non-ischemic dilated cardiomyopathy, ischemic infarcted cardiomyopathy, and hypertrophic cardiomyopathy with ages ranging from 22 to 74. Plasma from patients with cardiomyopathy contains elevated soluble PNC (sPNC), ranging from approximately 10 ng/ml to 30 ng/ml, while sPNC in healthy donor plasma averages approximately 2 ng/mL (p<0.0001, FIG. 7F). Two additional fibrotic conditions representative of lung and liver fibrosis were screened for sPNC. Expression of sPNC in plasma from IPF patients (n=9) and plasma from fibrosis-scored NAFLD-cirrhosis patients (n=12) is significantly elevated relative to healthy donors (p<0.05, FIG. 7F). We propose the detection of sPNC could allow for quantitative, non-invasive monitoring of fibrogenesis and disease progression. A large sample prospective study along with clinical analysis will be required for future studies.

### Fibronectin Is a Potential PNC Binding Partner

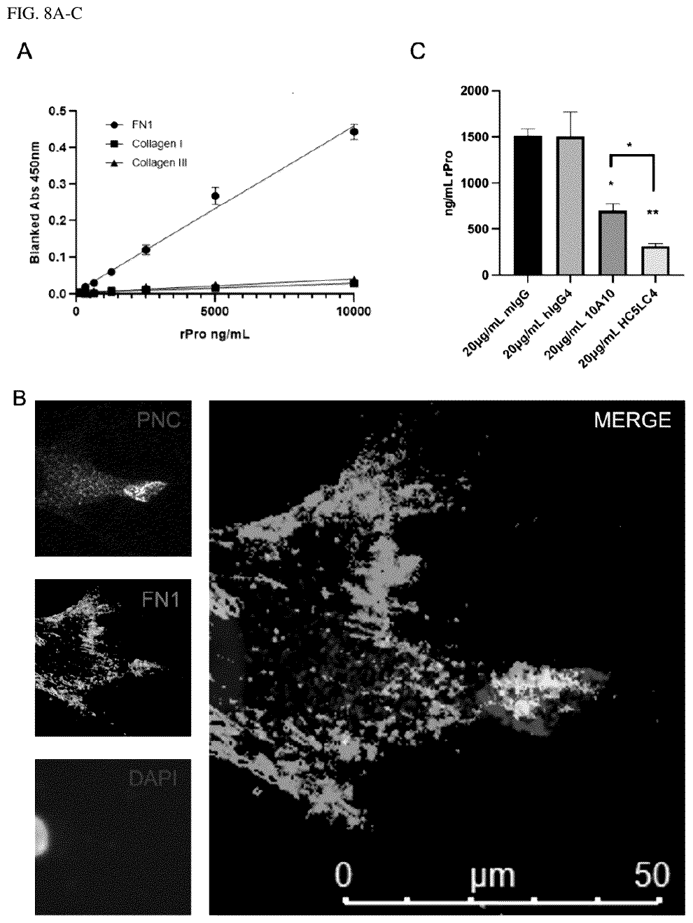

We hypothesize that the prodomain of N-cadherin may bind to an ECM protein component [Xiong, Y.; Liu, L.; Zhu, S.; Zhang, B.; Qin, Y.; Yao, R.; Zhou, H.; Gao, D. Precursor N-cadherin mediates glial cell line-derived neurotrophic factor-promoted human malignant glioma. Oncotarget 2017, 8, 24902-24914]. A common assay to detect binding partners was used to assess binding potential to three major ECM proteins overexpressed in fibrotic conditions. After immobilizing fibronectin, type I and type III collagen to a polystyrene, medium binding plate and blocking with BSA, recombinant his-tagged prodomain (rPro) peptide was incubated over the immobilized and blocked ECM proteins. A biotinylated murine α-his-tag antibody and streptavidin HRP was used for detection of bound rPro peptide. Binding activity of rPro peptide to immobilized fibronectin is shown; however, neither human type I collagen or type III collagen-coated wells efficiently capture rPro from solution (FIG. 8A). Subsequently, myofibroblasts isolated from pathological tissues were co-stained for PNC and FN1. Colocalization of PNC and FN1 is shown on cellular protrusions by immunofluorescent microscopy (FIG. 8B). Consistent with our binding study, this suggests FN1 is a potential binding partner to the prodomain of N-cadherin.

After observing the binding activity and colocalization of rPro and FN1, we hypothesized that an α-PNC antibody would interfere with this interaction. Fully humanized α-PNC antibody HC5LC4 (Fusion Antibodies, PLC), demonstrates an approximately 10-fold greater affinity to the recombinant prodomain peptide relative to murine 10A10 chimeric control (FIG. 1A). We therefore selected this clone to test this hypothesis. The FN1 solid phase ELISA protocol (FIG. 8A) was used to test m-α-PNC mAb 10A10 and fully humanized α-PNC mAb HC5LC4 (h-α-PNC mAb) blocking activity. Both humanized and murine α-PNC mAbs block rPro peptide from absorbing to immobilized fibronectin (FIG. 8C). There is no competition between epitopes for α-His-tag detection antibody and α-PNC antibody (FIG. 1B). Consistent with the K_{d} values generated by Fusion Antibodies (FIG. 1A), humanized α-PNC mAb HC5LC4 blocking activity is significantly greater than murine antibody 10A10 (FIG. 8C).

### Migration of PNC-Expressing Myofibroblasts is Inhibited by Recombinant Prodomain and Fully Humanized α-PNC Antibody

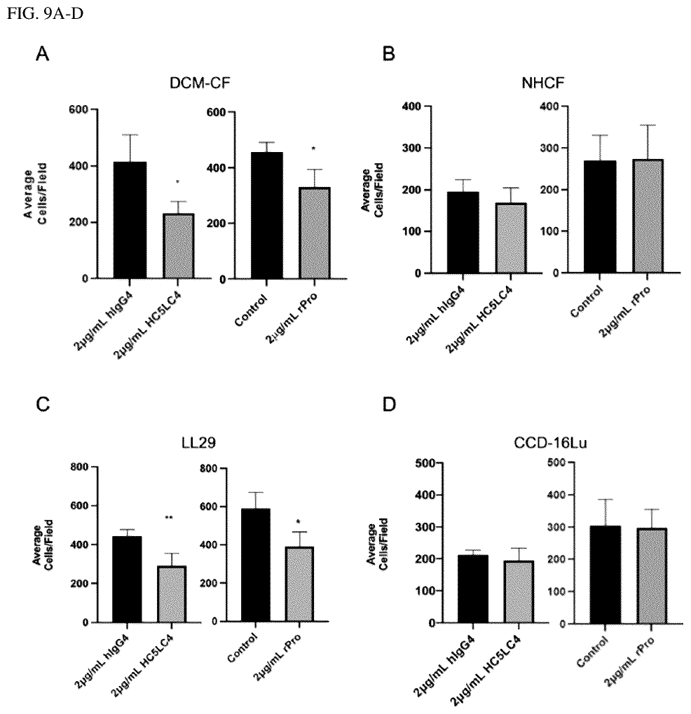

Given the evidence that PNC is a marker of carcinoma invasion, fibronectin is an inducer of cell motility, and PNC is localized to cellular protrusions on pathological myofibroblasts, we hypothesized that the disruption of the potential cell surface FN1/PNC interaction may prevent the migration of pathogenic PNC expressing myofibroblasts [Maret, D.; Gruzglin, E.; Sadr, M. S.; Siu, V.; Shan, W.; Koch, A. W.; Seidah, N. G.; Del Maestro, R. F.; Colman, D. R. Surface Expression of Precursor N-cadherin Promotes Tumor Cell Invasion. Neoplasia 2010, 12, 1066-IN38; Hsiao, C.-T.; Cheng, H.-W.; Huang, C.-M.; Li, H.-R.; Ou, M.-H.; Huang, J.-R.; Khoo, K.-H.; Yu, H. W.; Chen, Y.; Wang, Y.-K.; et al. Fibronectin in cell adhesion and migration via N-glycosylation. Oncotarget 2017, 8, 70653-70668]. PNC-positive myofibroblasts were subjected to a Boyden chamber migration assay in the presence of h-α-PNC mAb HC5LC4 or human IgG4 isotype control (hIgG4). We also hypothesized treatment with the rPro peptide would impair cell migration by competing with cell surface PNC for binding partners; therefore, treatment with rPro in solution was used as a cell surface PNC antagonist to corroborate the role of cell surface PNC in migration. Boyden chambers were first coated with human FN1 and TGF-β1 was added to the complete media used in the bottom chamber for a chemoattractant. Following a 5 h incubation, migration of PNC-expressing myofibroblasts is significantly impaired by both rPro peptide and h-α-PNC mAb HC5LC4 with no effect on normal myofibroblast cell lines (FIG. 9). Interestingly, the reduction in average cell numbers that passed through the membrane is consistent with the percent positivity of cell surface expressing PNC myofibroblasts demonstrated by earlier flow cytometry experiments (FIG. 4A). These data suggest that pathological myofibroblasts utilize cell surface PNC to migrate, and targeting cell surface PNC inhibits migration.

### Plasma Monitoring of PNC May be a Useful Clinical Tool

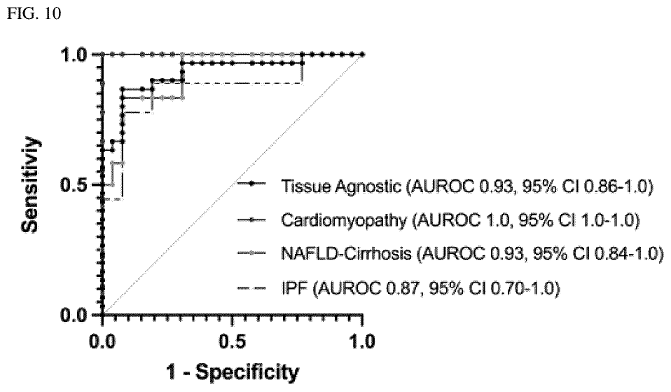

To evaluate the potential for plasma monitoring of PNC to serve as a biomarker for fibrosis development, ELISA-quantified plasma sampled were assessed using AUROC (area under receiver-operating characteristic) analysis and a 95% CI was obtained using the hybrid Wilson/Brown method. Samples were assessed for sensitivity and specificity on a tissue-specific (FIG. 10; red, yellow, green lines) and tissue-agnostic (FIG. 10; black line) basis. In each case, the AUROC is at least 0.87 (IPF) and in the case of cardiomyopathy, the AUROC is 1.0. While these data are encouraging, we caution that this is a small cohort of retrospective sampling, and further studies will be needed to validate the predictive value of plasma monitoring of PNC in patients with fibrosis.

### Discussion

Our study suggests the need to further investigate N-cadherin biology regarding tissue fibrosis, as reagents developed to study and/or target N-cadherin in this context may or may not cross-react with the pro-N-cadherin precursor protein. This study and others suggest PNC has a unique and divergent role from what is typical of mature N-cadherin [Herrera, A.; Menendez, A.; Torroba, B.; Ochoa, A.; Pons, S. Dbnl and β-catenin promote pro-N-cadherin processing to maintain apico-basal polarity. J. Cell Biol. 2021, 220; Maret, D.; Gruzglin, E.; Sadr, M. S.; Siu, V.; Shan, W.; Koch, A. W.; Seidah, N. G.; Del Maestro, R. F.; Colman, D. R. Surface Expression of Precursor N-cadherin Promotes Tumor Cell Invasion. Neoplasia 2010, 12, 1066-IN38; Nelson, E. R.; Li, S.; Kennedy, M.; Payne, S.; Kilibarda, K.; Groth, J.; Bowie, M.; Parilla-Castellar, E.; De Ridder, G.; Marcom, P. K.; et al. Chemotherapy enriches for an invasive triple-negative breast tumor cell subpopulation expressing a precursor form of N-cadherin on the cell surface. Oncotarget 2016, 7, 84030-84042; Xiong, Y.; Liu, L.; Zhu, S.; Zhang, B.; Qin, Y.; Yao, R.; Zhou, H.; Gao, D. Precursor N-cadherin mediates glial cell line-derived neurotrophic factor-promoted human malignant glioma. Oncotarget 2017, 8, 24902-24914]. In its mature form, N-cadherin functions as a cell-cell adhesion molecule [Wahl, J. K.; Kim, Y. J.; Cullen, J. M.; Johnson, K. R.; Wheelock, M. J. N-cadherin-Catenin Complexes Form Prior to Cleavage of the Proregion and Transport to the Plasma Membrane. J. Biol. Chem. 2003, 278, 17269-17276; Shan, W.-S.; Tanaka, H.; Phillips, G. R.; Arndt, K.; Yoshida, M.; Colman, D. R.; Shapiro, L. Functional Cis-Heterodimers of N- and R-Cadherins. J. Cell Biol. 2000, 148, 579-590]. In its precursor form, the data suggest that it may serve a role in cell-ECM focal adhesions. This is particularly interesting when considering the cardiac structure of the intercalated disc. The major cell adhesion molecule within the fascia adherens of the cardiac intercalated discs is N-cadherin [Kostetskii, I.; Li, J.; Xiong, Y.; Zhou, R.; Ferrari, V. A.; Patel, V. V.; Molkentin, J. D.; Radice, G. L. Induced Deletion of the N-cadheringene in the Heart Leads to Dissolution of the Intercalated Disc Structure. Circ. Res. 2005, 96, 346-354; Vite, A.; Radice, G. L. N-cadherin/catenin Complex as a Master Regulator of Intercalated Disc Function. Cell Commun. Adhes. 2014, 21, 169-179]. Mature N-cadherin is essential in maintaining the structure and function of the intercalated discs, as well as myofibrillar organization and myocyte shape [Chopra, A.; Tabdanov, E.; Patel, H.; Janmey, P. A.; Kresh, J. Y. Cardiac myocyte remodeling mediated by N-cadherin-dependent mechanosensing. Am. J. Physiol. Circ. Physiol. 2011, 300, H1252-H1266]. Conversely, loss of N-cadherin results in disassembly of intercalated disc structure in the mammalian heart, dilated cardiomyopathy, impaired cardiac function and death [Kostetskii, I.; Li, J.; Xiong, Y.; Zhou, R.; Ferrari, V. A.; Patel, V. V.; Molkentin, J. D.; Radice, G. L. Induced Deletion of the N-cadheringene in the Heart Leads to Dissolution of the Intercalated Disc Structure. Circ. Res. 2005, 96, 346-354; Li, J.; Patel, V. V.; Kostetskii, I.; Xiong, Y.; Chu, A. F.; Jacobson, J. T.; Yu, C.; Morley, G. E.; Molkentin, J. D.; Radice, G. L. Cardiac-Specific Loss of N-cadherin Leads to Alteration in Connexins With Conduction Slowing and Arrhythmogenesis. Circ. Res. 2005, 97, 474-481]. The biomechanical consequences of PNC localization to intercalated discs could play a role in the progression of heart failure.

Tissue fibrosis is observed anatomically as the accumulation of excessive extracellular matrix which stresses cell-cell contacts through increased tensile force and elicits maladaptive remodeling of tissues [Baum, J.; Duffy, H. S. Fibroblasts and Myofibroblasts: What Are We Talking About? J. Cardiovasc. Pharmacol. 2011, 57, 376-379; McCain, M. L.; Lee, H.; Aratyn-Schaus, Y.; Kléber, A. G.; Parker, K. K. Cooperative coupling of cell-matrix and cell-cell adhesions in cardiac muscle. Proc. Natl. Acad. Sci. USA 2012, 109, 9881-9886]. Cells can respond to increased mechanical load at the cell-cell junctions by forming focal adhesions to offload intercellular mechanical force and maintain tensional homeostasis [McCain, M. L.; Lee, H.; Aratyn-Schaus, Y.; Kléber, A. G.; Parker, K. K. Cooperative coupling of cell-matrix and cell-cell adhesions in cardiac muscle. Proc. Natl. Acad. Sci. USA 2012, 109, 9881-9886; Mertz, A. F.; Che, Y.; Banerjee, S.; Goldstein, J. M.; Rosowski, K. A.; Revilla, S. F.; Niessen, C. M.; Marchetti, M. C.; Dufresne, E. R.; Horsley, V. Cadherin-based intercellular adhesions organize epithelial cell-matrix traction forces. Proc. Natl. Acad. Sci. USA 2012, 110, 842-847; Mui, K. L.; Chen, C.; Assoian, R. K. The mechanical regulation of integrin-cadherin crosstalk organizes cells, signaling and forces. J. Cell Sci. 2016, 129, 1093-1100]. Conversely, it is also demonstrated that focal adhesions and cell-cell junctions can function inversely [Yano, H.; Mazaki, Y.; Kurokawa, K.; Hanks, S. K.; Matsuda, M.; Sabe, H. Roles played by a subset of integrin signaling molecules in cadherin-based cell-cell adhesion. J. Cell Biol. 2004, 166, 283-295]. In the heart, this is observed in developmental and pathological processes. Mimicking fibrosis in cardiac cells resulted in increased focal adhesion formation adjacent to the cell-cell interface [McCain, M. L.; Lee, H.; Aratyn-Schaus, Y.; Kléber, A. G.; Parker, K. K. Cooperative coupling of cell-matrix and cell-cell adhesions in cardiac muscle. Proc. Natl. Acad. Sci. USA 2012, 109, 9881-9886]. This was interpreted as the need for mechanical off-loading from the intercalated discs. In the pathological processes of cardiac fibrosis and remodeling, increased focal adhesion is correlated with lower working efficiency of the cardiac tissue [McCain, M. L.; Lee, H.; Aratyn-Schaus, Y.; Kléber, A. G.; Parker, K. K. Cooperative coupling of cell-matrix and cell-cell adhesions in cardiac muscle. Proc. Natl. Acad. Sci. USA 2012, 109, 9881-9886]. The observation of FN1 as a potential PNC binding partner leads us to postulate that post translational changes to N-cadherin processing, through retention of the prodomain could serve as a method for poised cellular response to changes in mechanical load. Future studies are required to understand the role of PNC at the cell surface in settings of fibrosis.

It is possible the role of PNC is cell dependent. In this study, PNC expression is observed in multiple different diseased tissues associated with fibrosis and tissue remodeling. Targeting PNC with a monoclonal antibody specific for an epitope on the prodomain results in reduction of migration by PNC expressing myofibroblasts suggesting a role for PNC in myofibroblast migration. This is consistent with the role of cell surface PNC in carcinogenesis and a recent report that cell surface PNC regulates apico-basal polarity in neural stem cells [Herrera, A.; Menendez, A.; Torroba, B.; Ochoa, A.; Pons, S. Dbnl and β-catenin promote pro-N-cadherin processing to maintain apico-basal polarity. J. Cell Biol. 2021, 220; Maret, D.; Gruzglin, E.; Sadr, M. S.; Siu, V.; Shan, W.; Koch, A. W.; Seidah, N. G.; Del Maestro, R. F.; Colman, D. R. Surface Expression of Precursor N-cadherin Promotes Tumor Cell Invasion. Neoplasia 2010, 12, 1066-IN38; Nelson, E. R.; Li, S.; Kennedy, M.; Payne, S.; Kilibarda, K.; Groth, J.; Bowie, M.; Parilla-Castellar, E.; De Ridder, G.; Marcom, P. K.; et al. Chemotherapy enriches for an invasive triple-negative breast tumor cell subpopulation expressing a precursor form of N-cadherin on the cell surface. Oncotarget 2016, 7, 84030-84042; Xiong, Y.; Liu, L.; Zhu, S.; Zhang, B.; Qin, Y.; Yao, R.; Zhou, H.; Gao, D. Precursor N-cadherin mediates glial cell line-derived neurotrophic factor-promoted human malignant glioma. Oncotarget 2017, 8, 24902-24914]. Cell surface PNC expression in vivo, but not mature N-cadherin, caused loss of apico-basal polarity, breach of basal membrane and invasion of neural precursors into the ventricle and surrounding mesenchyme of the neural tube [Herrera, A.; Menendez, A.; Torroba, B.; Ochoa, A.; Pons, S. Dbnl and β-catenin promote pro-N-cadherin processing to maintain apico-basal polarity. J. Cell Biol. 2021, 220]. The observation that rPro antagonizes cell migration suggests cell surface PNC is a mediator of pathological myofibroblast migration, as opposed to the sPNC. In progressive fibrosis, myofibroblasts continually migrate into and within the affected organ without resolving, leading to disease progression. This is readily apparent in cardiac fibrosis and IPF disease progression [Baum, J.; Duffy, H. S. Fibroblasts and Myofibroblasts: What Are We Talking About? J. Cardiovasc. Pharmacol. 2011, 57, 376-379; Karvonen, H. M.; Lehtonen, S.; Sormunen, R. T.; Harju, T. H.; Lappi-Blanco, E.; Bloigu, R. S.; Kaarteenaho, R. L. Myofibroblasts in interstitial lung diseases show diverse electron microscopic and invasive features. Lab. Investig. 2012, 92, 1270-1284]. In the case of cardiac fibrosis, myofibroblasts can be found widespread within the failing heart [Baum, J.; Duffy, H. S. Fibroblasts and Myofibroblasts: What Are We Talking About? J. Cardiovasc. Pharmacol. 2011, 57, 376-379]. It is thought that IPF is initiated at the periphery of the lungs and slowly migrates to encompass the entirety of the lungs [Wellman, T. J.; Mondoñedo, J. R.; Davis, G. S.; Bates, J. H. T.; Suki, B. Topographic distribution of idiopathic pulmonary fibrosis: A hybrid physics- and agent-based model. Physiol. Meas. 2018, 39, 064007]. It has been demonstrated that myofibroblasts isolated from advanced IPF display a higher capacity to migrate than those isolated from less advanced disease stages [Suganuma, H.; Sato, A.; Tamura, R.; Chida, K. Enhanced migration of fibroblasts derived from lungs with fibrotic lesions. Thorax 1995, 50, 984-989]. Targeting myofibroblasts migration is a reasonable therapeutic strategy for mitigating fibrotic conditions.

It has been postulated that cell surface prodomain cleavage from N-cadherin is a means of spatially and temporally regulating adhesion during development and synapse formation [Latefi, N. S.; Pedraza, L.; Schohl, A.; Li, Z.; Ruthazer, E. S. N-cadherin prodomain cleavage regulates synapse formationin vivo. Dev. Neurobiol. 2009, 69, 518-529]. Increased expression and proteolysis of the prodomain at the cell surface could explain elevated levels of sPNC found in the plasma of patients with pathological fibrosis. It could also be argued that increased cell death due to disease could contribute to the elevation of the 17 kDa prodomain in the plasma. Almost one third of the plasma proteome is made up of intercellular proteins that have escaped due to cellular turnover [Wildes, D.; Wells, J. A. Sampling the N-terminal proteome of human blood. Proc. Natl. Acad. Sci. USA 2010, 107, 4561-4566]. With the origins of the 60 kDa protein unknown, future studies are necessary to understand the processing of PNC in tissue fibrosis.

Our study is a preliminary report with a relatively limited and mostly retrospective sample set, highlighting the need for further studies to thoroughly evaluate PNC as both a biomarker and a therapeutic target in fibrotic disease. Additionally, our data (FIG. 2) indicate that epithelial cells in each respective tissue and not only the myofibroblasts may have an important role in PNC-driven fibrotic disease. This is important considering existing work that identifies PNC as a driver of migration and invasion in carcinogenesis [Maret, D.; Gruzglin, E.; Sadr, M. S.; Siu, V.; Shan, W.; Koch, A. W.; Seidah, N. G.; Del Maestro, R. F.; Colman, D. R. Surface Expression of Precursor N-cadherin Promotes Tumor Cell Invasion. Neoplasia 2010, 12, 1066-IN38; Nelson, E. R.; Li, S.; Kennedy, M.; Payne, S.; Kilibarda, K.; Groth, J.; Bowie, M.; Parilla-Castellar, E.; De Ridder, G.; Marcom, P. K.; et al. Chemotherapy enriches for an invasive triple-negative breast tumor cell subpopulation expressing a precursor form of N-cadherin on the cell surface. Oncotarget 2016, 7, 84030-84042].

The experiments described in this example identified PNC as a potential biomarker of fibrotic conditions, PNC was shown to be detectable in the plasma of patients with cardiomyopathy, IPF and NAFLD-cirrhosis in a quantifiable manner and we have suggested that aberrant proteolytic processing of N-cadherin is involved in the pathophysiology of fibrosis. Perhaps most importantly, PNC is expressed on the cell surface of myofibroblasts and potentially other cells within tissues in the pathological setting of fibrosis, but has not been observed on the surface of physiologically normal postnatal cell types [Wahl, J. K.; Kim, Y. J.; Cullen, J. M.; Johnson, K. R.; Wheelock, M. J. N-cadherin-Catenin Complexes Form Prior to Cleavage of the Proregion and Transport to the Plasma Membrane. J. Biol. Chem. 2003, 278, 17269-17276; Latefi, N. S.; Pedraza, L.; Schohl, A.; Li, Z.; Ruthazer, E. S. N-cadherin prodomain cleavage regulates synapse formationin vivo. Dev. Neurobiol. 2009, 69, 518-529; Maret, D.; Gruzglin, E.; Sadr, M. S.; Siu, V.; Shan, W.; Koch, A. W.; Seidah, N. G.; Del Maestro, R. F.; Colman, D. R. Surface Expression of Precursor N-cadherin Promotes Tumor Cell Invasion. Neoplasia 2010, 12, 1066-IN38]. This makes PNC an attractive candidate for a potential serological surrogate for fibrosis progression and potential therapeutic target that may offer specificity to failing tissues and the invasive myofibroblasts that drive disease. Further studies will be required to discern to what extent serological concentration of sPNC correlates with disease progression. In summary, pro-N-cadherin is aberrantly localized in all fibrotic tissues examined in this study and elevated in the plasma of all patients with fibrosis analyzed in this study.

### Example II

### Methods

### Study Participants

The study population included 690 participants within the MURDOCK Study community registry and biorepository (Bhattacharya S, Dunham A A, Cornish M A, Christian V A, Ginsburg G S, Tenenbaum J D, Nahm M L, Miranda M L, Califf R M, Dolor R J, et al. The Measurement to Understand Reclassification of Disease of Cabarrus/Kannapolis (MURDOCK) Study Community Registry and Biorepository. Am J Transl Res. 2012; 4:458-470). Collection of serum as part of the MURDOCK study has been described (Bhattacharya S, Dunham A A, Cornish M A, Christian V A, Ginsburg G S, Tenenbaum J D, Nahm M L, Miranda M L, Califf R M, Dolor R J, et al. The Measurement to Understand Reclassification of Disease of Cabarrus/Kannapolis (MURDOCK) Study Community Registry and Biorepository. Am J Transl Res. 2012; 4:458-470; The MURDOCK Study. ctsi.duke.edu/research-support/duke-kannapolis/murdock-study. 2022). All participants in the study population reported no heart failure at baseline, as indicated by response to the MURDOCK Study enrollment questionnaire. More information about the MURDOCK Study storefront is included in Supplementary Information. A control cohort with no reported heart failure at baseline and no reported heart failure over a 13-year follow up was identified (cohort A, n=289) from within the study population. A second cohort with no reported heart failure at baseline but later reported heart failure at any time during a 13-year follow up was identified (cohort B, n=307) from within the study population. A “low risk” subgroup of cohort A was identified by excluding participants with coronary artery disease (CAD), high blood pressure, previous heart attack, atrial fibrillation, or NTproBNP levels above the age dependent rule-in cutoffs for heart failure at enrollment. The age dependent rule-in consensus values of 450, 900, and 1800 pg/mL for ages <50, 50-75, and >75 respectively, were used in this study. A “high risk” subgroup of cohort B was identified that included participants that reported at least 1, 2, or 3 heart failure risk factors. Risk factors were defined as coronary artery disease, high blood pressure, or previous heart attack reported at enrollment. Where indicated, “low risk” subgroup A was compared to “high risk” subgroup B. Insufficient sample prevented measurement of NTproBNP in 6 samples from cohort B that were excluded from analysis.

The MURDOCK Community Registry and Biorepository and related ancillary studies are or must be approved by the institutional review boards of both Duke University Medical Center (Durham, NC) and Carolinas HealthCare System (Charlotte, NC). All patients provided written informed consent for the collection of biological samples and use of their clinical data. The current analyses were approved by the Duke University Medical Center Institutional Review Board.

### Enzyme-Linked Immunosorbent Assays

Detection and quantification of serum pro-N-cadherin by ELISA has been described (Ferrell P D, Oristian K M, Cockrell E, Pizzo S V. Pathologic Proteolytic Processing of N-Cadherin as a Marker of Human Fibrotic Disease. Cells. 2022; 11:156). Detection and quantification of serum NTproBNP was performed according to the manufacturer's recommendations (R&D Systems, DY3604-05).

### Statistics

GraphPad Prism version 9.4.0 (673) was employed for statistical analysis. Welch's T-test was used to evaluate significance of serum values between the two cohorts. ROC curves were calculated by Wilson/Brown method with 95% confidence intervals. Relationships between PNC, age, BMI, and NTproBNP were evaluated by simple linear regression. Welch's T-test was performed to evaluate differences of PNC values between male and female genders. Distribution of age, BMI, weight and blood pressure were evaluated using Mann-Whitey's test except diastolic blood pressure which was evaluated using Welch's test following a test of normality. Proportion race/ethnic groups were evaluated using Fisher's exact test. Survival curves with hazard ratios were generated using log-rank test. Adjusted hazard ratios for all cause mortality and development of heart failure were generated using Cox Proportional Hazards Ratio. (p<0.05*, p<0.01**, p<0.001***, p<0.0001****).

### Results

### Study Population and Definition of Cohorts

The study population comprised 690 participants from the Measurement to Understand Reclassification of Disease Of Cabarrus and Kannapolis (MURDOCK) Study at the Duke Clinical and Translational Science Institute²⁰. All participants in the study population enrolled at Time=0 as members of the general population with no known heart failure. They were subsequently followed for 1 to 13 years by a self-report style questionnaire in which any health conditions that developed were noted. cohort A (n=345) is defined by those who did not report the development of heart failure at any time following enrollment. Cohort B (n=345) is defined by those who indicated no known heart failure at time of enrollment and serum collection, (Time=0) but reported development of heart failure on a subsequent follow up. NTproBNP levels were used to corroborate heart failure status of each cohort. Cohorts were populated by the Duke Clinical and Translational Research Institute and reviewed for exclusion criteria by study investigators. Cohorts were further refined by the following exclusions: participants with no follow ups and participants who reported “yes”, “I don't know”, or “Null” to heart failure at baseline. Cohorts A and B hereafter will refer to cohorts after exclusion refinement based on the above criteria (Table 1). Population dynamics of Cohorts A and B after exclusion refinement do not differ significantly from those of the unrefined cohorts.

TABLE 1
Demographic summary of participants curated for study.

| | All Participants | | Cohort A | | Cohort B | | P value | NC^{!} |
|---|---|---|---|---|---|---|---|---|
| N | 690 | | 345 | | 345 | | | |
| N (after exclusion refinement) | 596 | | 289 | | 307 | | | |
| Age (years) | 68 | [22-95] | 68 | [27-95] | 68 | [22-95] | 0.7615 | |
| BMI | 30 | [16-82] | 28 | [17-60] | 29 | [16-82] | 0.0004 | |
| Weight (lbs) | 188 | [92-506] | 179 | [92-404] | 197 | [95-506] | 0.0010 | |
| Systolic Blood Pressure (mmHg) | 133 | [72-231] | 132 | [81-231] | 134 | [72-197] | 0.3162 | |
| Diastolic Blood Pressure (mmHg) | 75 | [44-126] | 76 | [52-126] | 74 | [44-120] | 0.0815 | |
| Male identified persons | 298 | (43%) | 149 | (43%) | 149 | (43%) | >.9999 | 49% |
| Female identified persons | 392 | (57%) | 196 | (57%) | 196 | (57%) | >.9999 | 51% |
| Hispanic | 15 | (2%) | 9 | (3%) | 6 | (2%) | 0.6032 | 12% |
| Black | 116 | (17%) | 58 | (17%) | 58 | (17%) | >.9999 | 21% |
| AAPI | 2 | (0%) | 0 | (0%) | 2 | (0.5%) | 0.4993 | 2% |
| Native American | 4 | (0.5%) | 2 | (0.5%) | 2 | (0.5%) | >.999 | 0% |
| Non-Hispanic White | 544 | (79%) | 275 | (80%) | 269 | (78%) | 0.6413 | 61% |
| Majority sample collection year* | 2011 | [′09-′16] | 2009 | [′09-′16] | 2011 | [′09-′16] | — | |
| Follow up period^{#} (years) | 7.6 ± 3 | [1-13] | 7.4 ± 3.5 | [1-13] | 7.8 ± 3.2 | [1-13] | — | |

| | | | | |
|---|---|---|---|---|
| Cardiovascular risk factors | 0.9 | 0.6 | 1.1 | <0.0001 |

Unless otherwise indicated, values are reported as: arithmetic mean [range]. Units, where applicable, are indicated in (parenthesis). Racial, ethnic, and gender identity data are reported as: ‘N’ (%).
^{!}For reference, US Census Bureau statistics for the 2010 Census in Kannapolis, NC are provided.
*Majority sample collection year is reported as: arithmetic mode of the calendar years in which samples were collected for participants in this study [range].
^{#}Follow up period is reported as: arithmetic mean ± standard deviation [range]. Cardiovascular risk factors is reported as arithmetic mean, given that each participant may have any combination of each of 3 pre-determined cardiovascular risk factors at time of sample collection: high blood pressure, prior heart attack, and coronary artery disease and presence of a given risk factor is weighted with a value of 1. P values are provided comparing the distribution (age, BMI, weight, blood pressure) or proportion (race, ethnicity) where appropriate between cohort A and cohort B using Mann-Whitney, Welch's, or Fisher's exact test. Significant differences (p < 0.05) are highlighted in bold.

### Population Dynamics of Cohorts A and B

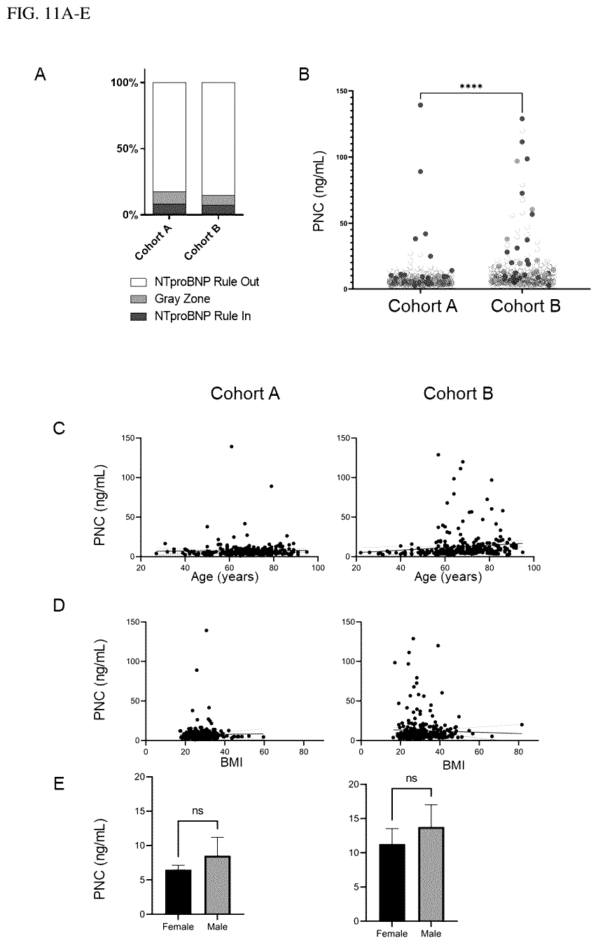

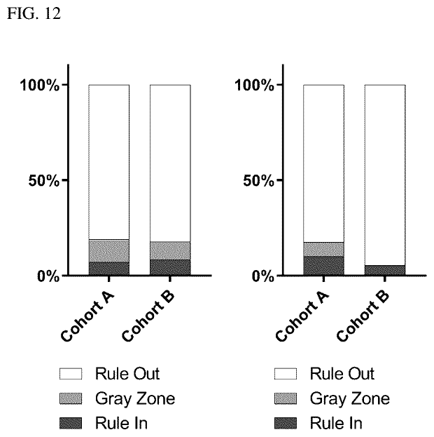

The median age of cohort A and B are 68 (27-95) and 68 (22-95), respectively. The ratio of male to female persons in each cohort was matched, 57 percent female and 43 percent male. Cohort B had higher reporting of high blood pressure; 50.7 percent of cohort A and 68.5 percent of cohort B reported high blood pressure at enrollment. However, there was no significant difference in measured mean systolic (cohort A 132.0 vs cohort B 134.0, p=0.2333) or diastolic (cohort A 76 vs cohort B 74, p=0.0815) blood pressure at the time of enrollment between the two cohorts. Cohort B reports overall higher cardiovascular risk factors. The mean BMI was slightly higher in cohort B at 29 (16-82) relative to cohort A at 28 (17-60). Both groups were well within the margin of error for parameters of NTproBNP levels expected from the general population with no diagnosis of heart failure. We selected a study-designated rule-out cut off of 300 pg/mL, consistent with a review of the NTproBNP literature and within the quantifiable limits of the assay utilized for this study²². Of cohort A, 82.4 percent were below the study-designated rule-out cut off <300 pg/mL NTproBNP for heart failure and 8.0 percent were above the age dependent cut offs previously listed. Of cohort B, 85.4 percent were below the rule-out cut off <300 pg/mL NTproBNP for heart failure and 7.3 percent were above the age dependent cut offs. Both cohorts show a similar proportion of expected rule-in and rule-out NTproBNP values (FIG. 11A,B; FIG. 12).

### Relationship of Soluble Pro-N-Cadherin to NTproBNP and Potential Confounding Variables

A relationship between PNC and age, sex, or body mass index (BMI), was analyzed. A simple linear regression suggests neither age nor BMI are correlated with PNC levels within either cohort (FIG. 11C,D). There is no significant difference in PNC levels between males and females from either cohort A (FIG. 11E, p=0.1436) or B (FIG. 1E, p=0.2121).

### Soluble Pro-N-Cadherin is a Biomarker of Subclinical Heart Failure

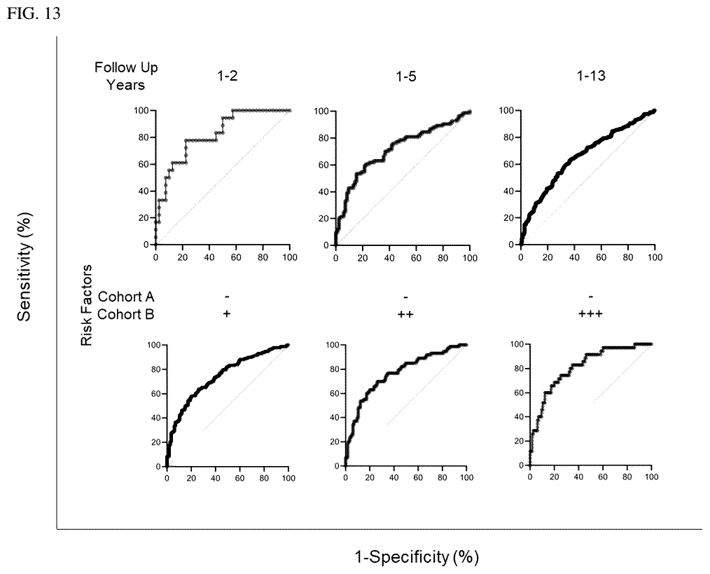

Participants that subsequently reported heart failure have significantly higher levels of soluble PNC in the serum relative to participants that did not subsequently report heart failure following enrollment (FIG. 11B). ROC analysis was performed using PNC values to determine diagnostic accuracy for subclinical heart failure. First, we analyzed each cohort with no exclusion criteria applies over follow-up periods of ≤13 years, ≤5 years and ≤2 years (Table 2; FIG. 13, top). We then performed ROC analysis of all follow up times, excluding participants in cohort A that meet the heart failure rule-in criteria for NTproBNP levels or reported any heart disease risk factors and compared them to participants that reported at least 1, 2, or 3 study-designated heart disease risk factors in cohort B (Table 2; FIG. 13, bottom). The majority of NTproBNP values fall below the quantifiable range of the NTproBNP assay, therefore ROC analysis was not indicated.

TABLE 2
ROC analyses of age, BMI, and cardiovascular risk factors relative to PNC. AUC,
p-value and change in AUC are reported for each subgroup: participants followed
1-13 years (1-13 yr F/U), participants followed 1-5 years (1-5 yr F/U) participants
followed 1-2 years (1-2 yr F/U) and number of cardiovascular risk factors (CV
Risks). AUC values are reported as AUC (95% confidence interval).

| | 1-13 yr F/U | 1-5 yr F/U | 1-2 yr F/U | CV Risks ≥ 1 | CV Risks ≥ 2 | CV Risks = 3 |
|---|---|---|---|---|---|---|
| AUC | | | | | | |
| Age | 0.5072 | 0.5485 | 0.5785 | 0.6440 | 0.7014 | 0.6884 |
| | (0.4608- | (0.4612- | (0.4201- | (0.5904- | (0.6283- | (0.6015- |
| | 0.5536) | 0.6359) | 0.7368) | 0.7076) | 0.7745) | 0.7754) |
| BMI | 0.5405 | 0.5837 | 0.5342 | 0.6689 | 0.6638 | 0.6678 |
| | (0.5001- | (0.4965- | (0.3567- | (0.6095- | (0.5835- | (0.5585- |
| | 0.5809) | 0.6709) | 0.7117) | 0.7283) | 0.7441) | 0.7770) |
| CV Risks | 0.6516 | 0.6304 | 0.6146 | | | |
| | (0.6081- | (0.5465- | (0.4483- | | | |
| | 0.6951) | 0.7142) | 0.7809) | | | |
| PNC | 0.6595 | 0.7158 | 0.8208 | 0.7422 | 0.7649 | 0.8127 |
| | (0.6160- | (0.6375- | (0.7089- | (0.6887- | (0.6942- | (0.7336- |
| | 0.7029) | 0.7941) | 0.9328) | 0.7957) | 0.8355) | 0.8917) |
| P-value | | | | | | |
| Age | 0.7614 | 0.2773 | 0.3423 | <0.0001 | <0.0001 | 0.0008 |
| BMI | 0.0466 | 0.0626 | 0.6803 | <0.0001 | 0.0002 | 0.0028 |
| CV Risks | <0.0001 | 0.0035 | 0.1656 | | | |
| PNC | <0.0001 | <0.0001 | 0.0001 | <0.0001 | <0.0001 | <0.0001 |
| ΔAUC | | | | | | |
| PNC vs Age | 0.1523 | 0.1673 | 0.2423 | 0.0982 | 0.0635 | 0.1243 |
| PNC vs BMI | 0.119 | 0.1321 | 0.2866 | 0.0733 | 0.1011 | 0.1449 |
| PNC vs CV | 0.0079 | 0.0854 | 0.2062 | | | |
| Risks | | | | | | |

Age, BMI, and number of defined cardiovascular risk factors were also analyzed by ROC curve for each subgroup (Table 2). The AUC, p-value and change in AUC relative to PNC was calculated for each subgroup analyzed. When binning for follow up times, AUC involving age, BMI, and number of cardiovascular risk factors remained relatively constant, however, diagnostic ability of PNC with shortened follow up times analyzed was enriched. Favorable risk discrimination for developing heart failure using PNC was observed at follow-up time of ≤2 years (p=. 0001, AUC 0.82 95% CI 0.71 to 0.93). Binning cohort B for number of cardiovascular risk factors and comparing those participants in cohort A with no risk factors enriched the predictability of Age and BMI, which is consistent with advancing age and higher BMI as risk factors for developing heart failure. These data suggest PNC has diagnostic value for subclinical heart failure.

### PNC is Positively Correlated to NTproBNP

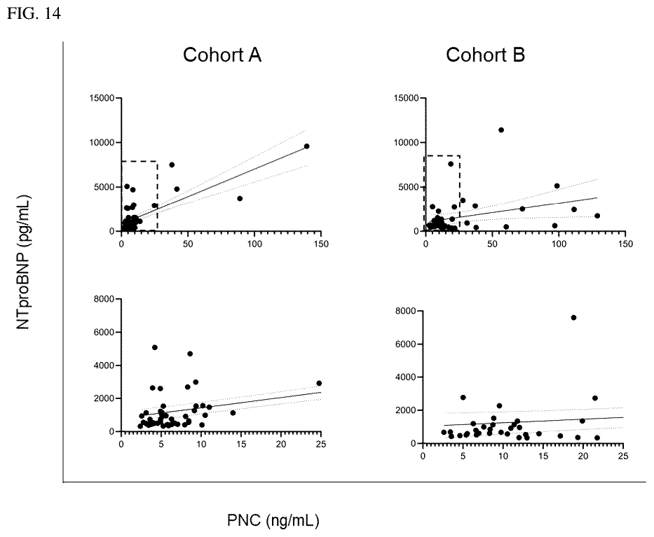

The relationship between PNC and NTproBNP in these cohorts was investigated. Participant's PNC and NTproBNP levels from each cohort were analyzed by simple linear regression using participants data whose NTproBNP values were within the range of the assay. Interestingly, a positive correlation between PNC and NTproBNP is found within cohort A (FIG. 14, left panels; Slope 62.16, r²=0.56); however, a weaker correlation is observed in cohort B (FIG. 14, right panels; Slope 21.41, r²=0.10). These data suggest a correlation between PNC and NTproBNP serum levels.

PNC Levels are Correlated with all-Cause Mortality

Survival curves were constructed using measured PNC or NTproBNP levels and the days post sample collection to the reported death dates or days post sample collection to the last follow up year recorded over a total of 13 years. Initially we compared the overall survival of cohort A to cohort B and as predicted, there was a significant reduction in the survival rate of cohort B relative to cohort A (FIG. 15A, p=0.0016, HR 1.64 95% CI 1.22 to 2.20). Then we assigned a PNC level threshold value of 6 ng/mL which falls between the median values for PNC levels of both cohorts. There is a significant reduction in 13-year survival rate for participants from combined cohorts A and B whose PNC level measures ≥6 ng/ml (FIG. 15B, p<0.0001, HR 1.99 95% CI 1.48 to 2.67). There is no significant difference between 13-year survival of participants within cohort A who measured above 6 ng/mL versus participants who measured below 6 ng/mL for PNC (FIG. 15C). However, there is a significant reduction in 13-year survival in participants that measure over 6 ng/mL in cohort B relative to those who measure under 6 ng/ml in cohort B (FIG. 4D, p<0.0001, HR 2.53 95% CI 1.74 to 3.69). As a combined cohort, we found no significant difference in survival between individuals that measured under 300 pg/mL NTproBNP and those who measured over 300 pg/mL (FIG. 15E). Although not significant, there is a reduction in survival for participants whose NTproBNP measures over 300 pg/mL in cohort A (FIG. 4F, p=0.093, HR 1.66 95% CI 0.82 to 3.33). There is no significant difference in survival for participants whose NTproBNP measures below vs above 300 pg/mL in cohort B (FIG. 15G). Additional analysis was performed using Cox Proportional Hazard Ratios. Age, BMI, and PNC are significant variables relative to all-cause mortality and development of heart failure after adjustment for all other risk covariates (Table 3). These data suggest PNC has diagnostic/prognostic value for subclinical heart failure within the general population.

TABLE 3
Cox proportional hazards regression analysis of combined cohorts (n =
596). Hazard ratios are adjusted for all other predictor variables and representative
of all-cause mortality (top) or development of heart failure (bottom).

| | Variable | Hazard Ratio | 95% CI | P value |
|---|---|---|---|---|
| All-Cause | Age | 1.098 | 1.078 to 1.119 | <0.0001 |
| Mortality Model | BMI | 1.037 | 1.009 to 1.064 | 0.0077 |
| | Gender (F) | 0.9452 | 0.6829 to 1.312 | 0.7348 |
| | PNC [≥6 ng/ml] | 1.414 | 1.014 to 1.993 | 0.044 |
| | NTproBNP [≥300 pg/mL] | 1.22 | 0.8032 to 1.794 | 0.33 |
| | High BP (Yes) | 1.231 | 0.8592 to 1.796 | 0.2676 |
| | Heart Attack (Yes) | 0.8792 | 0.5097 to 1.484 | 0.6362 |
| | CAD (Yes) | 1.276 | 0.7762 to 2.032 | 0.3205 |
| Heart Failure | Age | 1.008 | 0.9963 to 1.021 | 0.1764 |
| Model | BMI | 1.020 | 1.002 to 1.037 | 0.0232 |
| | Gender (F) | 1.228 | 0.9586 to 1.578 | 0.1060 |
| | PNC [≥6 ng/ml] | 1.555 | 1.213 to 2.002 | 0.0006 |
| | NTproBNP [≥300 pg/mL] | 0.9395 | 0.6623 to 1.300 | 0.7161 |
| | High BP (Yes) | 1.259 | 0.9639 to 1.655 | 0.0950 |
| | Heart Attack (Yes) | 1.663 | 1.092 to 2.516 | 0.0169 |
| | CAD (Yes) | 1.353 | 0.9043 to 1.988 | 0.1326 |

### Discussion

Studies consistently report the prognostic value of NTproBNP for patients with heart failure; by contrast, studies showing prognostic value of NTproBNP in individuals with subclinical heart failure in the general population are inconsistent. NTproBNP is a poor diagnostic tool for screening of subclinical heart failure in the general population in two recent studies of large cohorts (Mureddu G F, Tarantini L, Agabiti N, Faggiano P, Masson S, Latini R, Cesaroni G, Miceli M, Forastiere F, Scardovi A B, et al. Evaluation of different strategies for identifying asymptomatic left ventricular dysfunction and pre-clinical (stage B) heart failure in the elderly. Results from ‘PREDICTOR’, a population based-study in central Italy. European Journal of Heart Failure. 2013; 15:1102-1112; Averina M, Stylidis M, Brox J, Schirmer H. NT-ProBNP and high-sensitivity troponin T as screening tests for subclinical chronic heart failure in a general population. ESC Heart Failure. 2022; 9:1954-1962). NTproBNP was evaluated as a means to predict those with Stage B heart failure defined by 12-lead ECG and Doppler transthoracic echocardiogram from a healthy population and was found ineffective (AUC=0.566) (Mureddu G F, Tarantini L, Agabiti N, Faggiano P, Masson S, Latini R, Cesaroni G, Miceli M, Forastiere F, Scardovi A B, et al. Evaluation of different strategies for identifying asymptomatic left ventricular dysfunction and pre-clinical (stage B) heart failure in the elderly. Results from ‘PREDICTOR’, a population based-study in central Italy. European Journal of Heart Failure. 2013; 15:1102-1112; Averina M, Stylidis M, Brox J, Schirmer H. NT-ProBNP and high-sensitivity troponin T as screening tests for subclinical chronic heart failure in a general population. ESC Heart Failure. 2022; 9:1954-1962). In addition, NTproBNP was found to have no prognostic value in predicting overall survival in a long term follow up study with a large cohort of healthy participants (McKie P M, Cataliotti A, Lahr B D, Martin F L, Redfield M M, Bailey K R, Rodeheffer R J, Burnett J C, Jr. The prognostic value of N-terminal pro-B-type natriuretic peptide for death and cardiovascular events in healthy normal and stage A/B heart failure subjects. J Am Coll Cardiol. 2010; 55:2140-2147). In part, this lack of prognostic value can be attributed to a common single nucleotide polymorphism found within the promotor region of BNP that results in elevated BNP products in the blood (Costello-Boerrigter L C, Boerrigter G, Ameenuddin S, Mahoney D W, Slusser J P, Heublein D M, Redfield M M, Rodeheffer R J, Olson T M, Burnett J C. The Effect of the Brain-Type Natriuretic Peptide Single-Nucleotide Polymorphism rs198389 on Test Characteristics of Common Assays. Mayo Clinic Proceedings. 2011; 86:210-218). However, this does not fully explain the complexity of NTpro/BNP as a biomarker.

Another challenge for clinicians when considering Ntpro/BNP as part of heart failure diagnosis is the lack of standardization. While 100 pg/ml is a widely agreed upon rule-out concentration for BNP, there is otherwise considerable variability and a large “gray zone” (Semenov A G, Feygina E E. Chapter One—Standardization of BNP and N T-proBNP Immunoassays in Light of the Diverse and Complex Nature of Circulating BNP-Related Peptides. In: Makowski G S, ed. Advances in Clinical Chemistry; Mccullough P A, Kluger A Y. Interpreting the Wide Range of N T-proBNP Concentrations in Clinical Decision Making. J Am Coll Cardiol. 2018; 71:1201-1203). This is further complicated by lack of standardization between assays currently in use for clinical applications (Semenov A G, Feygina E E. Chapter One—Standardization of BNP and N T-proBNP Immunoassays in Light of the Diverse and Complex Nature of Circulating BNP-Related Peptides. In: Makowski G S, ed. Advances in Clinical Chemistry; Mccullough P A, Kluger A Y. Interpreting the Wide Range of N T-proBNP Concentrations in Clinical Decision Making. J Am Coll Cardiol. 2018; 71:1201-1203). Despite this, in one of the most cited studies describing NTproBNP, the age dependent rule-in consensus values of 450, 900, and 1800 pg/mL for ages <50, 50-75, and >75 respectively, yielded 90% sensitivity and 84% specificity for acute heart failure (Hill S A, Booth R A, Santaguida P L, Don-Wauchope A, Brown J A, Oremus M, Ali U, Bustamam A, Sohel N, Mckelvie R. Use of BNP and N T-proBNP for the diagnosis of heart failure in the emergency department: a systematic review of the evidence. Heart failure reviews. 2014; 19:421-438). The consensus <300 pg/mL had a negative predictive value of 98% in the same study (Hill S A, Booth R A, Santaguida P L, Don-Wauchope A, Brown J A, Oremus M, Ali U, Bustamam A, Sohel N, McKelvie R. Use of BNP and N T-proBNP for the diagnosis of heart failure in the emergency department: a systematic review of the evidence. Heart failure reviews. 2014; 19:421-438). While this is helpful, there is a clear unmet need for a biomarker to identify patients at risk for developing heart failure prior to onset of symptoms.

Finally, nTpro/BNP must also be evaluated through the lens of other comorbidities and physiological variables that are known to raise or lower peptide concentrations. Standard nTpro/BNP levels are significantly different between races and dependent on BMI (Parcha V, Patel N, Kalra R, Arora G, Januzzi J L, Felker G M, Wang T J, Arora P. Racial Differences in Serial N T-proBNP Levels in Heart Failure Management. Circulation. 2020; 142:1018-1020; Myhre P L, Claggett B, Yu B, Skali H, Solomon S D, Røsjø H, Omland T, Wiggins K L, Psaty B M, Floyd J S, et al. Sex and Race Differences in N-Terminal Pro-B-type Natriuretic Peptide Concentration and Absolute Risk of Heart Failure in the Community. JAMA Cardiol. 2022; 7:623-631; Gupta D K, Lemos JAd, Ayers C R, Berry J D, Wang T J. Racial Differences in Natriuretic& #xa0; Peptide& #xa0; Levels. JACC: Heart Failure. 2015; 3:513-519; Vergaro G, Gentile F, Meems L M G, Aimo A, Januzzi J L, Richards A M, Lam C S P, Latini R, Staszewsky L, Anand I S, et al. NT-proBNP for Risk Prediction in Heart Failure. JACC: Heart Failure. 2021; 9:653-663). Advancing age, female sex, renal dysfunction, atrial fibrillation, and inflammation are characteristics contributing to high serum NTproBNP, while obesity leads to low serum concentration, which can make interpretation difficult (Bachmann K N, Gupta D K, Xu M, Brittain E, Farber-Eger E, Arora P, Collins S, Wells Q S, Wang T J. Unexpectedly Low Natriuretic Peptide Levels in Patients With Heart Failure. JACC: Heart Failure. 2021; 9:192-200; Marie R, Philippe M. B-type natriuretic peptide and obesity in heart failure: a mysterious but important association in clinical practice. Cardiovascular Medicine. 2020; Nishikimi T, Nakagawa Y. Potential pitfalls when interpreting plasma BNP levels in heart failure practice. J Cardiol. 2021; 78:269-274; Wang T J, Larson M G, Levy D, Leip E P, Benjamin E J, Wilson P W, Sutherland P, Omland T, Vasan R S. Impact of age and sex on plasma natriuretic peptide levels in healthy adults. Am J Cardiol. 2002; 90:254-258). Approximately 50 percent of heart failure cases are classified as heart failure with preserved ejection fraction (hFpEF) in which a majority of patients maintain normal natriuretic peptide levels (Sorimachi H, Omote K, Obokata M, Reddy Y, Borlaug B A. Abstract 9890: HFpEF with Normal Natriuretic Peptides: An Earlier Stage of Disease or Fundamentally Different Phenotype? Circulation. 2021; 144: A9890-A9890; Verbrugge F H, Omote K, Reddy Y N V, Sorimachi H, Obokata M, Borlaug B A. Heart failure with preserved ejection fraction in patients with normal natriuretic peptide levels is associated with increased morbidity and mortality. European Heart Journal. 2022; 43:1941-1951; Dunlay S M, Roger V_{L}, Redfield M M. Epidemiology of heart failure with preserved ejection fraction. Nat Rev Cardiol. 2017; 14:591-602). Taken together, these factors are particularly problematic when considering medically at-risk populations who more often face challenges being correctly diagnosed and having access to appropriate care. Including other biomarkers, such as PNC, has the opportunity to improve the efficiency and accuracy of care particularly when confounding variables are present. Future studies are needed to elucidate relationships between PNC and confounding variables not explored in this study and to determine the usefulness of PNC as a potential biomarker for hFpEF.

BNP and NTproBNP have proven to be sufficient biomarkers for ruling in and ruling out heart failure in patients already presenting with dyspnea; however, there is a clear need for biomarkers that predict heart failure earlier in disease progression to allow for intervention before remodeling becomes irreversible. This is evidenced by the increased mortality and incidence of sudden death associated with subclinical heart dysfunction (Hillis G S, Møller J E, Pellikka P A, Gersh B J, Wright R S, Ommen S R, Reeder G S, Oh J K. Noninvasive estimation of left ventricular filling pressure by E/e′ is a powerful predictor of survival after acute myocardial infarction. Journal of the American College of Cardiology. 2004; 43:360-367; Møller J E, Egstrup K, Køber L, Poulsen S H, Nyvad O, Torp-Pedersen C. Prognostic importance of systolic and diastolic function after acute myocardial infarction. Am Heart J. 2003; 145:147-153).

In our study, NTproBNP levels greater than 300 pg/mL were not prognostic for survival (FIG. 15). In part, this may be due to the relatively low incidence of heart failure risk factors at baseline in these participants (Table 1, Cardiovascular Risk Factors). These data suggest that PNC is elevated in the serum during early cardiac remodeling with predictive value for heart failure independent of existing comorbidities. Therefore, it could be used to identify patients who would benefit from preventative or early interventional therapy. Of note, 6 ng/ml PNC in the serum was not predictive of 13-year survival in the control cohort. Given that our previous studies indicated that fibrosis in other organs such as the lungs and liver result in serum PNC in the range of 4-6 ng/ml (Ferrell P D, Oristian K M, Cockrell E, Pizzo S V. Pathologic Proteolytic Processing of N-Cadherin as a Marker of Human Fibrotic Disease. Cells. 2022; 11:156). It is possible that serum PNC levels of various magnitudes could be used to discern fibrosis origin. This also suggests that a certain threshold of PNC in the serum is necessary to become predictive of heart failure and diverge from that of other pathologies. Further work is needed to determine the utility of serum PNC prognostication in other pathologies.

Serum PNC is indicative of the aberrant processing, localization, and solubilization of PNC from the cell surface observed in pathological tissue remodeling and fibrosis (Ferrell P D, Oristian K M, Cockrell E, Pizzo S V. Pathologic Proteolytic Processing of N-Cadherin as a Marker of Human Fibrotic Disease. Cells. 2022; 11:156). Our findings indicate that PNC is detectable in serum prior to the stage at which tissue fibrosis and remodeling produces increased cardiac wall tension and elevated NTpro/BNP. This suggests PNC could be used as a predictive screening biomarker for subclinical heart failure of at-risk individuals within the general population. Our data indicate that a community-based screening approach of individuals measuring greater than 8.13 ng/mL PNC results in a sensitivity of 77.8% and specificity of 77.5% that these individuals will be diagnosed with heart failure within 2 years. Additionally, we found that individuals with serum PNC levels greater than or equal to 6 ng/ml have a 41% increased chance of all-cause mortality after adjusting for age, gender, BMI, NTproBNP level, presence of high blood pressure, heart attack and coronary artery disease. Taken together, these data suggest PNC is a practical screening tool to identify individuals with cardiovascular risk factors commonly found in the general population and older individuals that will progress to heart failure. There is no established biomarker known to the literature that is predictive of heart failure independent of age, sex, BMI or comorbidities that can be used clinically as a community-based screening tool for subclinical heart failure (Averina M, Stylidis M, Brox J, Schirmer H. NT-ProBNP and high-sensitivity troponin T as screening tests for subclinical chronic heart failure in a general population. ESC Heart Failure. 2022; 9:1954-1962; Welsh P, Campbell R T, Mooney L, Kimenai D M, Hayward C, Campbell A, Porteous D, Mills N L, Lang N N, Petrie M C, et al. Reference Ranges for N T-proBNP (N-Terminal Pro-B-Type Natriuretic Peptide) and Risk Factors for Higher N T-proBNP Concentrations in a Large General Population Cohort. Circulation: Heart Failure. 2022; 15). Since this is the first report with evidence of serum PNC as a biomarker for subclinical heart failure, more studies will be necessary to establish clinical cut-offs and practical utility of serum PNC as a biomarker of subclinical heart failure in the clinical setting.

It is important to note that this study was limited by the nature of a self-reporting study. Limited information pertaining to clinical factors and comorbidities was available. While our data indicates that PNC adds predictive value to the study-designated cardiovascular risk factors, survival was analyzed based on all-cause mortality and could not be definitively attributed to cardiovascular related death. No echocardiogram data was available to exclude participants with cardiac structural anomalies or asymptomatic heart disease from cohort A. Furthermore, with only one blood collection (at time of enrollment in the study) the dynamics of serum PNC over time could not be evaluated over the decade or more of participant follow ups. Nonetheless, the significance of the ability to detect elevated PNC in a population with no self-reported heart failure prior to the onset of diagnostic symptoms should not be understated. Future studies are warranted to establish the prognostic potential of soluble PNC in a prospective manner.

### Example III

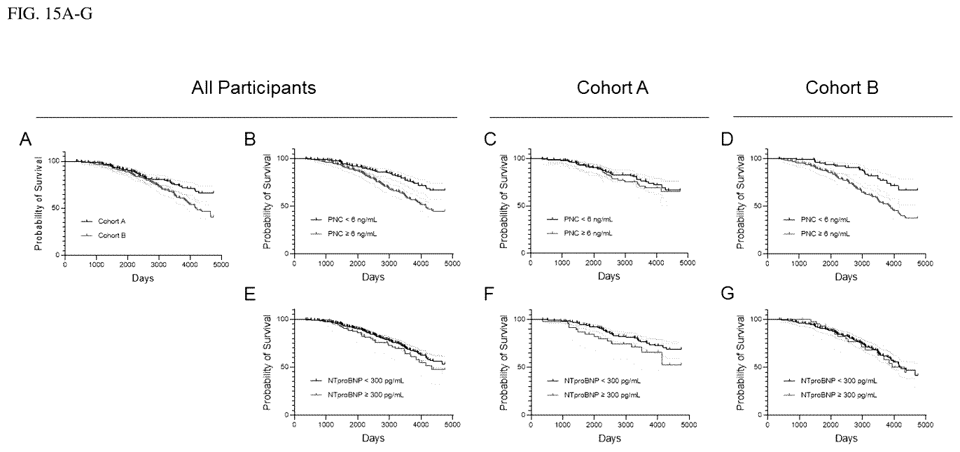

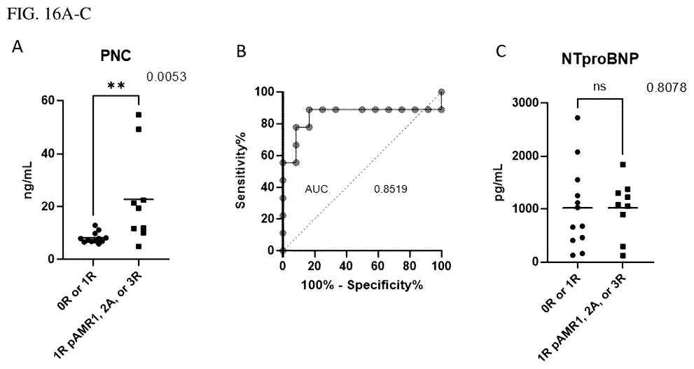

This example demonstrates that pro-N-Cadherin is a marker of cardiac transplant rejection. Post-operative human plasma samples were analyzed by previously described ELISA for PNC and compared to NTproBNP. PNC was shown to be significantly elevated in post cardiac transplant patients that progress to rejection stages 1R pAMR1, 2A or 3R compared to patients with no evidence of cardiac transplant rejection or IR with mild rejection and no evidence of cell damage (p=0.0053) (FIG. 16A). FIG. 16B shows a ROC curve representation of A (AUC=0.8519). No significant difference was observed in NTproBNP levels between the two cohorts (p=0.8078) (FIG. 16C).

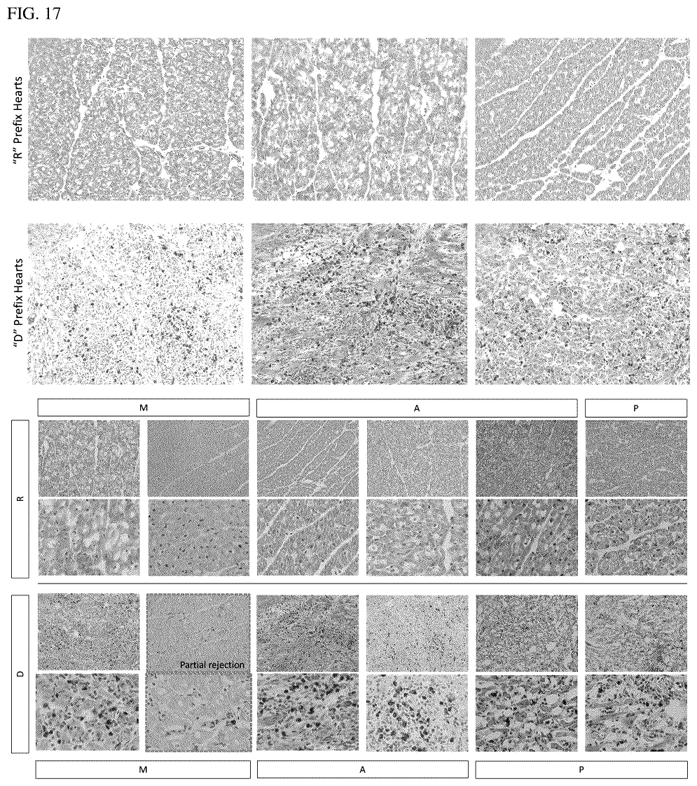

Additional experiments demonstrated that PNC is a marker of rejection in a porcine model of cardiac transplant. All tissues were stained with anti-PNC antibody by IHC. As shown in FIG. 17, “R” prefix designated non-rejected porcine cardiac tissues and “D” prefix designated rejected porcine cardiac tissues. A, M, and P designated anterior, middle, and posterior, respectively, relative to the biopsy's location within the interventricular septum sample. Partially rejected tissue is outlined and labeled within FIG. 17. Stacked images within the same row represent different magnifications within the same sample.

### Example IV

This example demonstrates that pro-N-Cadherin is a predictor of a subset of left ventricular assist device (LVAD) non-responders. LVAD responders were defined by patients that maintained an increased ejection fraction by at least 10 percent post LVAD placement. Human plasma samples were analyzed by previously described ELISA for PNC and compared to NTproBNP. PNC was elevated in a subset of patients that do not respond to LVAD therapy (p=0.0536) (FIG. 18A). No significant difference was observed in NTproBNP levels between the two cohorts (p=0.8078) (FIG. 18B).

### Example V

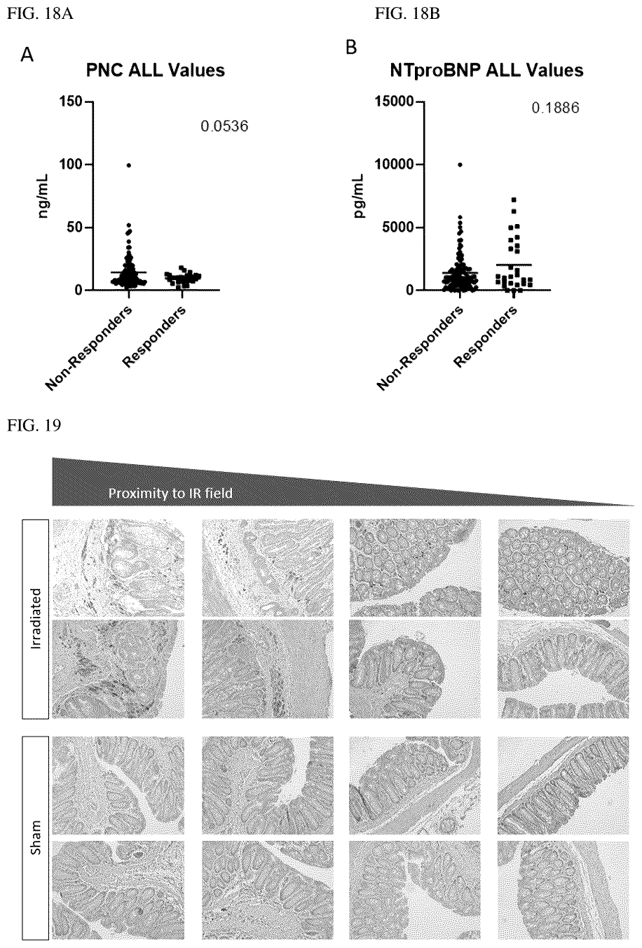

This example demonstrates that pro-N-cadherin is a marker of radiation induced fibrosis. FIG. 19 shows irradiated vs sham mouse colon stained with mouse-anti-mouse pro-N-cadherin antibody. Brown stain indicates cells positive for pro-N-cadherin protein. IR field consisted of approximately 2 cm of the rectum and distal colon starting from the anus (first 2 to 3 rings). FFPE tissues were stained using 10-20 μg/ml anti-pro-N-cadherin antibody.

### Example VI

This example demonstrates efficacy of anti-PNC monoclonal antibody therapy in doxorubicin-induced heart failure.

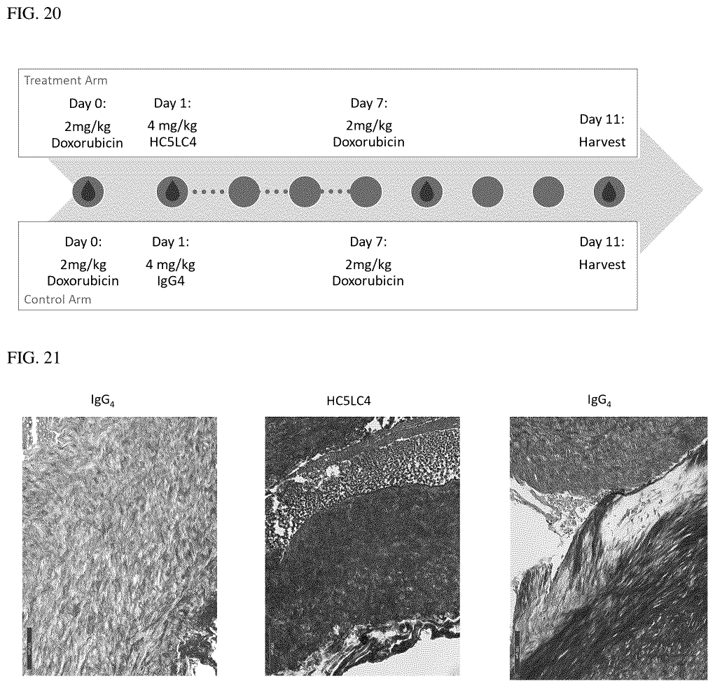

FIG. 20 shows a graphical depiction of the experimental design evaluating the efficacy of anti-PNC monoclonal antibody therapy in doxorubicin-induced heart failure. Heart failure was induced by doxorubicin injection at Day 0, followed by a single antibody or IgG control administration 24 hours after initiation of doxorubicin treatment. A second doxorubicin injection was administered 7 days following initiation. Blood was collected at days 0 (pre-treatment), 1 (pre-treatment), 8 (post-treatment), and at termination.

FIG. 21 shows representative images of reduced aortic stenosis in doxorubicin induced heart failure guinea pigs followed by treatment with anti-pro-N-cadherin antibody HC5LC4. Blue stain is indicative of collagen fibers, a hallmark of cardiac remodeling.

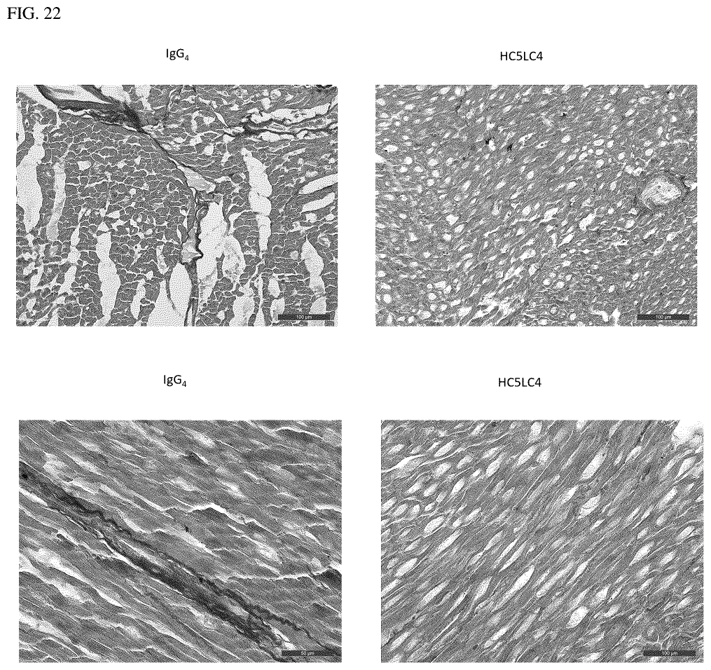

FIG. 22 shows representative images of reduced cardiac myocyte hypertrophy and interstitial collagen in doxorubicin-induced heart failure followed by treatment with anti-pro-N-cadherin antibody HC5LC4 as compared to control in guinea pigs.

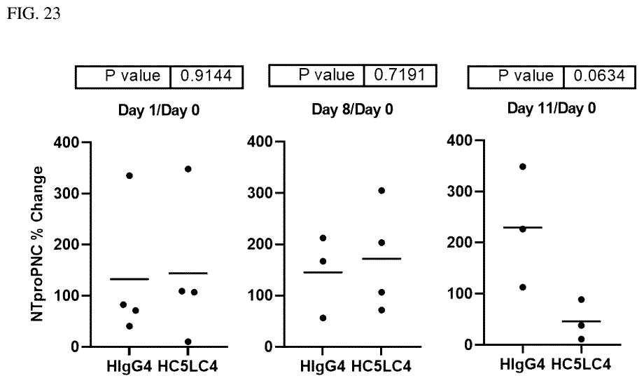

NTproBNP was quantified in serum samples from guinea pigs at day 0, 1, 8 and 11. Percent change from baseline, day 0, was calculated for each animal and compared between treatment groups. NTproBNP is directly correlated with cardiac wall stress (see, FIG. 23).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of characterizing a sample, the method comprising:
a) assaying for the presence of aberrant PNC cellular localization and/or PNC circulation in blood in a sample (e.g., a blood sample, a plasma sample, a serum sample, a whole blood sample, a buffy coat sample) obtained from a human subject; and
b) characterizing the sample as i) having pathological fibroblast cells if the presence of aberrant PNC cellular localization and/or PNC circulation in blood are detected in the assaying step, or ii) not having pathological fibroblast cells if the presence of aberrant PNC cellular localization and/or PNC circulation in blood are not detected in the assaying step.

2. The method of claim 1, wherein the assaying comprises use of an antibody against pro-N-cadherin.

3. The method of claim 2, wherein the antibody against pro-N-cadherin is HC5LC4.

4. The method of claim 1, wherein the assaying comprises use of a microarray chip having one or more nucleic acid molecules that can hybridize under stringent conditions to a nucleic acid molecule encoding fibroblasts expressing pro-N-cadherin or having one or more polypeptides (such as peptides or antibodies) that can bind to fibroblasts expressing pro-N-cadherin.

5. The method of claim 1, wherein a presence of pathological fibroblast cells indicates the human subject is experiencing fibrosis and/or a condition associated with fibrosis; wherein the condition associated with fibrosis is one or more conditions selected from respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma; chronic liver disease, primary biliary cirrhosis (PBC), schistosomal liver disease, liver cirrhosis; cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Chron's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease; diseases of the eye such as Grave's ophthalmopathy, epiretinal fibrosis, retinal fibrosis, subretinal fibrosis (e.g. associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis; arthritis; fibrotic pre-neoplastic and fibrotic neoplastic disease; fibrosis induced by chemical or environmental insult (e.g., cancer chemotherapy, pesticides, radiation/cancer radiotherapy); fibrosis induced by transplant (e.g., organ transplant) rejection; and fibrosis induced by non-response of medically implanted device (e.g., left ventricular assist device (LVAD)).

6. The method of claim 1, further comprising administering to the subject an anti-fibrotic therapeutic agent capable of inhibiting expression and/or activity related to pro-N-cadherin expression and/or activity.

7. The method of claim 6, wherein the anti-fibrotic therapeutic agent is a small molecule, a polypeptide or peptide fragment, an siRNA, or an antibody or fragment thereof

8. The method of claim 7, wherein the anti-fibrotic therapeutic agent is an antibody against fibroblasts expressing pro-N-cadherin and/or an antibody against pro-N-cadherin.

9. The method of claim 7, wherein the anti-fibrotic therapeutic agent is the anti-pro-N-cadherin antibody 10A10 and/or the anti-pro-N-cadherin antibody HC5LC4.

10. The method of claim 1, further comprising administering to the subject one or more additional therapeutic agents selected from an anti-IL-13 agent, an anti-IL-4 agent, a combination anti-IL-13/anti-IL-4 agent, pirfenidone, anti-LOXL2 antibody (GS-6624), N-acetylcysteine, anti-TGF-.beta. antibody (GC1008), anti-.alpha.v.beta.6 integrin antibody (STX-100), anti-CTGF antibody (FG-3019), anti-CCL2 antibody (CNTO 888), somatostatin analog (SOM230, octreotide), antiotensin II inhibitor (losartan), carbon monoxide, thalidomide, tetrathiomolybdate, doxycycline, minocycline, and tyrosine kinase inhibitor (BIBF1120).

11. A method for treating, ameliorating, and/or preventing a condition associated with pathological fibroblast cells expressing pro-N-cadherin in a subject, comprising administering to a human subject an anti-fibrotic therapeutic agent capable of inhibiting expression and/or activity related to aberrant PNC cellular localization and/or PNC circulation in blood (e.g., a blood, plasma, serum, a whole blood, buffy coat).

12. The method of claim 11, wherein the anti-fibrotic therapeutic agent is a small molecule, a polypeptide or peptide fragment, an siRNA, or an antibody or fragment thereof

13. The method of claim 12, wherein the anti-fibrotic therapeutic agent is an antibody against fibroblasts expressing pro-N-cadherin and/or an antibody against pro-N-cadherin.

14. The method of claim 12, wherein the anti-fibrotic therapeutic agent is the anti-pro-N-cadherin antibody 10A10 or wherein the anti-fibrotic therapeutic agent is the anti-pro-N-cadherin antibody HC5LC4.

15. The method of claim 11, wherein the condition associated with pathological fibroblast cells expressing pro-N-cadherin is selected from fibrosis, respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma; chronic liver disease, primary biliary cirrhosis (PBC), schistosomal liver disease, liver cirrhosis; cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Chron's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease; diseases of the eye such as Grave's ophthalmopathy, epiretinal fibrosis, retinal fibrosis, subretinal fibrosis (e.g. associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis; arthritis; fibrotic pre-neoplastic and fibrotic neoplastic disease; and fibrosis induced by chemical or environmental insult (e.g., cancer chemotherapy, pesticides, radiation/cancer radiotherapy); fibrosis induced by transplant (e.g., organ transplant) rejection; and fibrosis induced by non-response of medically implanted device (e.g., left ventricular assist device (LVAD)).

16. The method of claim 15, further comprising administering to the subject one or more additional therapeutic agents selected from an anti-IL-13 agent, an anti-IL-4 agent, a combination anti-IL-13/anti-IL-4 agent, pirfenidone, anti-LOXL2 antibody (GS-6624), N-acetylcysteine, anti-TGF-.beta. antibody (GC1008), anti-.alpha.v.beta.6 integrin antibody (STX-100), anti-CTGF antibody (FG-3019), anti-CCL2 antibody (CNTO 888), somatostatin analog (SOM230, octreotide), antiotensin II inhibitor (losartan), carbon monoxide, thalidomide, tetrathiomolybdate, doxycycline, minocycline, and tyrosine kinase inhibitor (BIBF1120).


SEQUENCE LISTING (1 sequence)

SEQ ID NO: 1 - protein, 159 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKR